(19) United States
(12) Patent Application Publication
(10) Pub. No.: US 2026/0055448 A1
(43) Pub. Date: Feb. 26, 2026

(54) HAIRPIN INTERNAL CONTROL FOR ISOTHERMAL NUCLEIC ACID AMPLIFICATION

(71) Applicant: Becton, Dickinson and Company, Franklin Lakes, NJ (US)

(72) Inventors: Honghua Zhang, San Diego, CA (US); Janay Elise Kong, San Diego, CA (US); Sina Rafizadeh, San Diego, CA (US)

(21) Appl. No.: 19/104,695

(22) PCT Filed: Sep. 6, 2023
(86) PCT No.: PCT/US2023/073519

§ 371 Date: Feb. 18, 2025

Related U.S. Application Data
(60) Provisional application No. 63/374,835, filed on Sep. 7, 2022.

(51) Int. Cl. C12Q 1/6853; C12Q 1/6876

(52) U.S. Cl. CPC C12Q 1/6853; C12Q 1/6876; C12Q 2600/166

(57) ABSTRACT

Disclosed herein include methods, compositions, and kits for monitoring an amplification reaction. Some embodiments include a quality control template comprising a 5′ subdomain, a 3′ subdomain, and a loop domain situated between the 5′ subdomain and the 3′ subdomain. Intramolecular nucleotide base pairing between the 5′ subdomain and the 3′ subdomain are capable of forming a paired stem domain. A quality control primer can be capable of hybridizing to at least a portion of the 3′ subdomain. Subjecting the quality control template and the quality control primer to an amplification reaction can generate a first quality control product. The method can comprise detecting the first quality control product.

### RELATED APPLICATIONS

This application is a National Stage application of PCT Application PCT/US2023/073519, filed Sep. 6, 2023, which claims the benefit under 35 U.S.C. § 119(e) of U.S. Provisional Patent Application Ser. No. 63/374,835, filed Sep. 7, 2022, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 68EB-317350-WO, created Sep. 6, 2023, which is 23,044 bytes in size. The information in the electronic format of the Sequence Listing is incorporated herein by reference in its entirety.

### BACKGROUND

### Field

The present disclosure relates generally to methods and compositions for amplification (e.g., isothermal amplification) of nucleic acids.

### Description of the Related Art

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products. Often, nucleic acid-based diagnostic assays require amplification of a specific portion of nucleic acid in a sample. A common technique for nucleic acid amplification is the polymerase chain reaction (PCR). This technique typically requires a cycling of temperatures (i.e., thermocycling) to proceed through the steps of denaturation (e.g., separation of the strands in the double-stranded DNA (dsDNA) complex), annealing of oligonucleotide primers (short strands of complementary DNA sequences), and extension of the primer along a complementary target by a polymerase. Such thermocycling can be a time consuming process that generally requires specialized machinery. Thus, a need exists for quicker nucleic acid amplification methods that can be performed without thermocycling. Such methods may be useful, for example, for on-site testing and point-of-care diagnostics. In particular, there is a need for compositions and methods of quicker nucleic acid detection wherein internal control(s) (“IC”) do not interfere with target amplification efficiency. Two main IC strategies have been employed in nucleic acid detection assays: competitive IC approaches and noncompetitive IC approaches. Their difference lies in whether the IC shares one common set of primers for IC and target amplification. With the competitive IC strategy there is always some competition between target and IC with the simultaneous amplification of target and IC fragments flanked by the same primers. The competition by IC amplification can lower target amplification efficiency and thereby result in a lower detection limit. Competitive IC methods therefore require more IC optimization to achieve a sensitive detection limit. In the noncompetitive approach, the target and IC are amplified using a different primer set for each. Although the kinetics of each reaction are not influenced by a competition for the primers, the IC amplification must be limited by a controlled concentration of the IC specific primers and/or IC template to limit the competition between the target and the IC reactions for primers and DNA polymerase. Therefore, nucleotide composition, copy number, and size of the IC must be carefully considered with this approach. There is a need for compositions and methods of monitoring an amplification reaction having reduced assay complexity and reduced undesirable interactions of the IC with target amplification.

### SUMMARY

Disclosed herein include methods for monitoring an amplification reaction. In some embodiments, the method comprises providing: a quality control template comprising: a 5′ subdomain; a 3′ subdomain; and a loop domain situated between the 5′ subdomain and the 3′ subdomain, wherein intramolecular nucleotide base pairing between the 5′ subdomain and the 3′ subdomain are capable of forming a paired stem domain. In some embodiments, the method comprises providing: a quality control primer capable of hybridizing to at least a portion of the 3′ subdomain. In some embodiments, the method comprises: subjecting the quality control template and the quality control primer to an amplification reaction capable of generating a first quality control product. In some embodiments, the method comprises: detecting the first quality control product. In some embodiments, the amplification reaction is conducted in an amplification reaction mixture under an amplification condition (e.g., an isothermal amplification condition). In some embodiments, subjecting the quality control template and the quality control primer to an amplification reaction capable of generating a first quality control product comprises: amplifying the quality control template with the quality control primer in the amplification reaction mixture under the amplification condition, thereby generating the first quality control product. In some embodiments, the amplification reaction comprises a reverse transcription reaction.

In some embodiments, the method further comprises: providing an enzyme having a polymerase activity (e.g., an enzyme having a hyperthermophile polymerase activity; and/or providing a reverse transcriptase. In some embodiments, the enzyme having a hyperthermophile polymerase activity has a reverse transcriptase activity. In some embodiments, the amplification reaction comprises: contacting the quality control primer with the quality control template for hybridization, and extending the quality control primer hybridized to the quality control template with an enzyme having a polymerase activity, thereby generating a first quality control product. In some embodiments, the amplification reaction comprises: contacting the quality control primer with the first quality control product for hybridization, and extending the quality control primer hybridized to the first quality control product with an enzyme having a polymerase activity, thereby generating a second quality control product. In some embodiments, the amplification reaction comprises: contacting the quality control primer with the second quality control product for hybridization, and extending the quality control primer hybridized to the second quality control product with an enzyme having a polymerase activity, thereby generating a first quality control product. In some embodiments, the first quality control product and second quality control product comprise a 5′ subdomain and the 3′ subdomain capable of forming a paired stem domain. In some embodiments, the first quality control product and second quality control product have the same stem domain. In some embodiments, the first quality control product and the second first quality control product comprise a loop domain complementary to each other. In some embodiments, the amplification reaction comprises linear and/or exponential amplification the first quality control product and the second quality control product. In some embodiments, the 5′ subdomain comprises the sequence of at least a portion of the quality control primer. In some embodiments, the first quality control product and the second quality control product are both capable of forming a hairpin structure. In some embodiments, the quality control template comprises a 5′ terminal domain situated 5′ of the 5′ subdomain, and/or the quality control template comprises a 3′ terminal domain situated 3′ of the 3′ subdomain. In some embodiments, the 5′ terminal domain of the quality control template comprises at least a portion of the sequence of the quality control primer. In some embodiments, the combined sequence of the 5′ terminal domain and the 5′ subdomain comprises the entire sequence of the quality control primer.

Detecting the first quality control product can comprise detecting the first quality control product with a signal-generating oligonucleotide. In some embodiments, the signal-generating oligonucleotide is capable of hybridizing to the first quality control product. In some embodiments, the detecting comprises contacting the first quality control product with the signal-generating oligonucleotide for hybridization. In some embodiments, the signal-generating oligonucleotide comprises a quencher, a label, or both. In some embodiments, the label comprises a comprises a quenchable label (e.g., a fluorophore). In some embodiments, the signal-generating oligonucleotide comprises a quencher. In some embodiments, the quencher is capable of quenching the label. In some embodiments, the detecting comprises contacting the first quality control product with the signal-generating oligonucleotide for hybridization. In some embodiments, the label is capable of generating a signal upon the signal-generating oligonucleotide hybridizing the first quality control product. In some embodiments, upon the signal-generating oligonucleotide hybridizing the first quality control product, the label generates a signal. In some embodiments, the signal is fluorescence. In some embodiments, detecting the first quality control product comprises detecting a signal generated by the label of the signal-generating oligonucleotide. In some embodiments, the label is a fluorophore and the signal is fluorescence. In some embodiments, the detecting comprises detecting the signal of the label before the amplification reaction, during the amplification reaction, after the amplification reaction, or any combination thereof.

In some embodiments, the method further comprises: providing a signal-generating oligonucleotide; subjecting the signal-generating oligonucleotide to the amplification reaction; and detecting the first quality control product with the signal-generating oligonucleotide. In some embodiments, the quality control template is a signal-generating oligonucleotide. In some embodiments, the quality control template is (i) a template for the synthesis of the first quality control product, and (ii) a means of detecting the first quality control product. In some embodiments, the signal-generating oligonucleotide is capable of (i) detecting the first quality control product and (ii) being a template for the quality control primer-driven synthesis of the first quality control product.

In some embodiments, the 5′ terminal domain of the quality control template comprises: one or more RNA nucleotides; and/or the sequence of at least a portion of the quality control primer. In some embodiments, the quality control template does not comprise a 3′ terminal domain; and/or the 3′ end of the quality control template is complementary to the 5′ end of the 5′ subdomain of the quality control template. In some embodiments, a reverse transcriptase is capable using the one or more RNA nucleotides of the 5′ terminal domain of the quality control template as a template to extend the 3′ end of the quality control template, thereby generating an extended quality control template. In some embodiments, the 3′ end of the extended quality control template comprises a sequence complementary to at least a portion of the quality control primer. In some embodiments, the amplification reaction comprises contacting a reverse transcriptase with the quality control template, thereby generating an extended quality control template. In some embodiments, the extended quality control template comprises cDNA. In some embodiments, the amplification reaction comprises: contacting the quality control primer with the 3′ end of the extended quality control template for hybridization, and extending the quality control primer hybridized to the 3′ end of the extended quality control template with a reverse transcriptase and/or an enzyme having a polymerase activity, thereby generating a first quality control product.

In some embodiments, the quality control template is a signal-generating oligonucleotide, wherein the signal-generating oligonucleotide comprises a label, and wherein the loop domain comprises one or more RNA nucleotides. In some embodiments, the label comprises a quenchable label (e.g., a fluorophore). In some embodiments, the signal-generating oligonucleotide comprises a quencher. In some embodiments, the label is situated in the 3′ terminal domain and the quencher is situated in the 5′ terminal domain, and/or the label is situated in the 5′ terminal domain and the quencher is situated in the 3′ terminal domain. In some embodiments, the amplification reaction comprises: contacting the quality control primer with the quality control template for hybridization, and extending the quality control primer hybridized to the quality control template with a reverse transcriptase, thereby generating a first quality control product. In some embodiments, the reverse transcriptase comprises RNaseH activity. In some embodiments, the reverse transcriptase cleaves the quality control template at the one or more RNA nucleotides during the generation of the first quality control product, thereby generating a first cleavage product comprising a label and a second cleavage product. In some embodiments, detecting the first quality control product comprises detecting a signal generated by the first cleavage product comprising a label. In some embodiments, the label is a fluorophore and the signal is fluorescence. In some embodiments, the method further comprises: providing a supplemental quality control primer; and subjecting the supplemental quality control primer to the amplification reaction.

In some embodiments, the signal-generating oligonucleotide is about 10 nucleotides to about 100 nucleotides in length; the quality control template is about 10 nucleotides to about 100 nucleotides in length; the quality control primer and/or the supplemental quality control primer is about 5 nucleotides to about 25 nucleotides in length; and/or the 5′ subdomain, the 3′ subdomain, the loop domain, the 5′ terminal domain, and/or the 3′ terminal domain is about 1 nucleotide to about 25 nucleotides in length. In some embodiments, the signal-generating oligonucleotide, the quality control template, and/or the quality control primer comprises one or more phosphorothioate linkages and/or one or more locked nucleic acids (LNAs). In some embodiments, the signal-generating oligonucleotide is a TaqMan detection probe oligonucleotide, a molecular beacon detection probe oligonucleotide, or a molecular torch detection probe oligonucleotide.

The signal-generating oligonucleotide can comprise one or more LNAs. In some embodiments, the one or more LNAs are situated within the loop domain (e.g., the one or more LNAs enhance the detectability of the first quality control product) and/or stem loop. In some embodiments, the signal-generating oligonucleotide is configured such that the melting temperature (Tm) of first quality control product/signal-generating oligonucleotide duplex is equal to or greater than the melting temperature (Tm) of the paired stem domain of the signal-generating oligonucleotide (e.g., configured via one or more LNAs situated in the loop domain).

In some embodiments, the signal-generating oligonucleotide does not comprise a dye capable of quenching the label. In some embodiments, the signal-generating oligonucleotide does not comprise a moiety capable of quenching the label other than the nucleotides of said signal-generating oligonucleotide. In some embodiments, the 5′ terminal domain of the quality control template and/or the signal-generating oligonucleotide comprises the label. In some embodiments, the 5′ terminal domain and/or the 5′ subdomain of the quality control template and/or the signal-generating oligonucleotide comprises one or more cytosine bases. In some embodiments, the 3′ terminal domain and/or the 3′ subdomain of the quality control template and/or the signal-generating oligonucleotide comprises one or more guanine bases and/or adenine bases. In some embodiments, the one or more guanine bases and/or adenine bases are capable of quenching the label upon the quality control template and/or the signal-generating oligonucleotide forming a hairpin structure. In some embodiments, the 3′ terminal domain of the quality control template and/or the signal-generating oligonucleotide comprises the label. In some embodiments, the 3′ terminal domain and/or the 3′ subdomain of the quality control template and/or the signal-generating oligonucleotide comprises one or more cytosine bases. In some embodiments, the 5′ terminal domain and/or the 5′ subdomain of the quality control template and/or the signal-generating oligonucleotide comprises one or more guanine bases and/or adenine bases. In some embodiments, the one or more guanine bases and/or adenine bases are capable of quenching the label upon the quality control template and/or the signal-generating oligonucleotide forming a hairpin structure.

In some embodiments, detecting the first quality control product comprises detecting a reduction in the amount of signal generated by a label of the quality control primer. In some embodiments, the label is a fluorophore and the signal is fluorescence. In some embodiments, the generation of the first quality control product and second quality control product is correlated with a decline in the signal detected. In some embodiments, the 5′ end of the quality control primer comprises a label. In some embodiments, the quality control primer comprises one or more pyrimidine bases adjacent to the label. In some embodiments, the quality control primer comprises one or more cytosine bases adjacent to the label. In some embodiments, the 3′ terminal domain and/or the 3′ subdomain of the quality control template, first quality control product, and/or the second quality control product comprises one or more guanine bases and/or adenine bases. In some embodiments, upon the quality control primer binding the quality control template and/or second quality control product and being extended with an enzyme having a polymerase activity to generate a first quality control product, the one or more guanine bases and/or adenine bases present in the 3′ terminal domain and/or the 3′ subdomain of the first quality control product are capable of quenching the label upon first quality control product forming a hairpin structure. In some embodiments, upon the quality control primer binding the first quality control product and being extended with an enzyme having a polymerase activity to generate a second quality control product, the one or more guanine bases and/or adenine bases present in the 3′ terminal domain and/or the 3′ subdomain of the second quality control product are capable of quenching the label upon second quality control product forming a hairpin structure. In some embodiments, detecting the first quality control product comprises contacting the first quality control product with a fluorescence dye.

In some embodiments, providing the quality control primer, the quality control template, and/or the signal-generating oligonucleotide comprises providing a reagent composition comprising the quality control primer, the quality control template, and/or the signal-generating oligonucleotide. In some embodiments, subjecting the quality control primer, the quality control template, and/or the signal-generating oligonucleotide to an amplification reaction comprises contacting the reagent composition with a treated sample to generate the amplification reaction mixture. In some embodiments, the method further comprises detecting a target nucleic acid sequence in a sample. In some embodiments, the method comprises: subjecting the target nucleic acid sequence to an amplification reaction capable of generating a nucleic acid amplification product. In some embodiments, the method comprises: detecting the nucleic acid amplification product with a target signal-generating oligonucleotide, wherein the target signal-generating oligonucleotide is capable of hybridizing to the nucleic acid amplification product. In some embodiments, subjecting the target nucleic acid sequence to an amplification reaction capable of generating a nucleic acid amplification product comprises: amplifying the target nucleic acid sequence in the amplification reaction mixture under the amplification condition, thereby generating a nucleic acid amplification product.

The method can comprise: contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence. The method can comprise: contacting the reagent composition with the treated sample to generate the amplification reaction mixture, wherein the reagent composition comprises one or more amplification reagents. In some embodiments, the one or more amplification reagents comprise: a reverse transcriptase; an enzyme having a hyperthermophile polymerase activity (e.g., an enzyme having a hyperthermophile polymerase activity and a reverse transcriptase activity); a forward primer; a reverse primer; a reverse transcription primer; and/or dNTPS. In some embodiments, the sample nucleic acids comprise a nucleic acid comprising the target nucleic acid sequence. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and ii) an enzyme having a hyperthermophile polymerase activity, thereby generating the nucleic acid amplification product. In some embodiments, the nucleic acid is: a double-stranded DNA; and/or a product of reverse transcription reaction. In some embodiments, the nucleic acid is a product of reverse transcription reaction generated from sample ribonucleic acids (e.g., the amplifying comprises generating the nucleic acid by a reverse transcription reaction).

In some embodiments, the amplification reaction is performed for a period of about 5 minutes to about 60 minutes. In some embodiments, amplifying the quality control template comprises generating the first quality control product and/or the second quality control product at detectable levels within about 20 minutes, about 15 minutes, or about 10 minutes. In some embodiments, amplifying the target nucleic acid sequence comprises generating the nucleic acid amplification product at detectable levels within about 20 minutes, about 15 minutes, or about 10 minutes. In some embodiments, the detecting is performed in less than about 20 minutes, less than about 15 minutes, less than about 10 minutes, less than about 5 minutes, or less than about 2 minutes, from the time the reagent composition is contacted with the treated sample. In some embodiments, the lysis buffer comprises one or more of magnesium sulfate, ammonium sulfate, EDTA, and EGTA. In some embodiments, the pH of the lysis buffer is about 1.0 to about 10.0. In some embodiments, the pH of the lysis buffer is about 2.2. In some embodiments, the sample nucleic acids comprise sample ribonucleic acids and/or sample deoxyribonucleic acids. In some embodiments, the sample nucleic acids comprise cellular RNA, mRNA, microRNA, bacterial RNA, viral RNA, or a combination thereof.

In some embodiments, the reagent composition is lyophilized, heat-dried, and/or comprises one or more additives, wherein the one or more additives comprise: Tween 20, Triton X-100, and/or tween 80; an amino acid; a sugar or sugar alcohol (e.g., sucrose, lactose, trehalose, dextran, erythritol, arabitol, xylitol, sorbitol, mannitol, or any combination thereof); and/or a polymer (e.g., polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof). In some embodiments, contacting the reagent composition with the treated sample comprises dissolving the reagent composition in the treated sample. In some embodiments, the one or more lytic reagents comprise: about 0.001% (w/v) to about 1.0 (w/v) of the treated sample (e.g., about 0.2% (w/v)); and/or a detergent (e.g., a cationic surfactant, an anionic surfactant, a non-ionic surfactant, an amphoteric surfactant). In some embodiments, the method: is performed in a single reaction vessel; does not comprise using any enzymes other than the reverse transcriptase and the enzyme having a hyperthermophile polymerase activity; does not comprise using any enzyme other than the enzyme having a hyperthermophile polymerase activity; does not comprise heat denaturing and/or enzymatic denaturing the nucleic acid and/or quality control template during the amplification step; and/or does not comprise contacting the nucleic acid and/or quality control template with a signal-stranded DNA binding protein.

In some embodiments, target nucleic acid sequence comprises a length of no longer than about 20 nucleotides to no longer than about 90 nucleotides. In some embodiments, the target nucleic acid sequence comprises a length of about 30 nucleotides. In some embodiments, the forward primer, the reverse primer, and/or the reverse transcription primer is about 8 to 16 bases long. In some embodiments, the nucleic acid amplification product is about 20 to 40 bases long. In some embodiments, the spacer sequence comprises a portion of the target nucleic acid sequence. In some embodiments, the spacer sequence is 1 to 10 bases long. In some embodiments, the isothermal amplification condition comprises a constant temperature of about 30° C. to about 72° C. (e.g., about 55° C. to about 75° C., about 56° C. to about 67° C.). In some embodiments, the amplifying is performed: for a period of about 5 minutes to about 60 minutes (e.g., about 15 minutes); and/or in helicase-free, signal-stranded binding protein-free, cleavage agent-free, and recombinase-free, isothermal amplification conditions.

In some embodiments, the enzyme having a hyperthermophile polymerase activity has an amino acid sequence that is at least about 90% identical to the amino acid sequence of SEQ ID NO: 1 or a functional fragment thereof. In some embodiments, the enzyme having a hyperthermophile polymerase activity has an amino acid sequence that is at least about 95% identical to the amino acid sequence of SEQ ID NO: 1. In some embodiments, the enzyme having a hyperthermophile polymerase activity has low or no exonuclease activity. In some embodiments, the sample ribonucleic acids are contacted with the reverse transcriptase and the enzyme having a hyperthermophile polymerase activity simultaneously. In some embodiments, the sample ribonucleic acids are contacted with the reverse transcriptase, the enzyme having a hyperthermophile polymerase activity, and the forward and reverse primers simultaneously. In some embodiments, the sample ribonucleic acids are contacted with the reverse transcriptase, the enzyme having a hyperthermophile polymerase activity, the forward primer, the reverse primer, and the reverse transcription primer simultaneously.

In some embodiments, the biological entities comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. In some embodiments, the target nucleic acid sequence is a nucleic acid sequence of a virus, bacteria, fungi, or protozoa. In some embodiments, the sample nucleic acids are derived from a virus, bacteria, fungi, or protozoa. In some embodiments, the sample is a biological sample or an environmental sample. In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

In some embodiments, the amplifying comprises multiplex amplification of two or more target nucleic acid sequences, and the detecting step comprises multiplex detection of two or more nucleic acid amplification products derived from said two or more target nucleic acid sequences. In some embodiments, the two or more target nucleic acid sequences are specific to two or more different organisms (e.g., one or more of SARS-CoV-2, Influenza A, Influenza B, and/or Influenza C). In some embodiments, the amplifying comprises one or more of Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise loop-mediated isothermal amplification (LAMP). In some embodiments, the method does not comprise one or more of: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

In some embodiments, the method, the reagent composition, and/or the amplification reaction mixture does not comprise: a template capable of generating the first quality control product other than the quality control template; a probe capable of detecting of the first quality control product other than the signal-generating oligonucleotide; a double-stranded template capable of generating the first quality control product; a linear template capable of generating the first quality control product; and/or a primer capable of hybridizing to the quality control template, the first quality control product and/or the second quality control product other than the quality control primer.

In some embodiments, the method comprises determining the presence, absence and/or amount of the first quality control product. In some embodiments, the presence, absence and/or amount of the signal indicates the presence, absence and/or amount of the first quality control product. In some embodiments, the presence, absence and/or amount of the signal indicates the presence, absence and/or amount of one or more interfering components in the amplification reaction mixture. In some embodiments, the presence, absence and/or amount of the signal indicates: (i) the integrity of the one or more amplification reagents in the amplification reaction mixture; (ii) failure of the instrument wherein the amplification reaction is conducted; and/or (iii) sample-derived inhibition of the amplification reaction. In some embodiments, sample-derived inhibition comprises matrix-derived inhibition. In some embodiments, the presence, absence and/or amount of the signal indicates the degree to which the amplification of the target nucleic acid sequence is inhibited in the amplification reaction.

In some embodiments, the method, the reagent composition, and/or the amplification reaction mixture comprises at least about 50,000 copies to about 500,000 copies of the quality control template. In some embodiments, a comparable method of monitoring an amplification reaction employs about 20 copies to about 100 copies of an internal control template. In some embodiments, the method, the reagent composition, and/or the amplification reaction mixture comprises an at least about 1.1-fold greater number of copies of the quality control template and/or quality control primer as compared to a comparable method of monitoring an amplification reaction that does not comprise the quality control template and/or the quality control primer. In some embodiments, said comparable method comprises an internal control template that is not capable of forming a hairpin structure. In some embodiments, a comparable method of monitoring an amplification reaction that does not comprise the quality control template and/or the quality control primer inhibits the amplification of the target nucleic acid sequence and/or the detection of nucleic acid amplification product by at least about 1.1-fold more than a method disclosed herein. In some embodiments, the quality control template and/or the quality control primer is not capable of hybridizing to the target nucleic acid sequence. In some embodiments, the presence of the quality control template and/or the quality control primer does not inhibit the amplification of the target nucleic acid sequence and/or the detection of nucleic acid amplification product. In some embodiments, the presence of the quality control template and/or the quality control primer in the amplification reaction mixture improves the amplification of the target nucleic acid sequence and/or detection of the nucleic acid amplification product by at least about 1.1-fold as compared to a comparable method wherein the quality control template and/or the quality control primer is absent from the amplification reaction mixture. In some embodiments, the number of false priming events and/or the generation of primer-dimers is reduced by at least about 1.1-fold as compared to a comparable method of monitoring an amplification reaction that does not comprise the quality control template and/or the quality control primer.

Disclosed herein include kits for monitoring an amplification reaction. In some embodiments, the kit comprises: a quality control template disclosed herein; a quality control primer disclosed herein; a signal-generating oligonucleotide disclosed herein; and/or a supplemental quality control primer disclosed herein.

The kit can comprise: a lysis buffer comprising one or more lytic agents capable of lysing biological entities to release sample nucleic acids comprised therein, wherein the sample nucleic acids are suspected of comprising a target nucleic acid sequence. In some embodiments, the one or more lytic agents comprise a detergent, and the detergent comprises one or more of a cationic surfactant, an anionic surfactant, a non-ionic surfactant, and an amphoteric surfactant. The kit can comprise: a reagent composition comprising one or more amplification reagents comprising one or more components for amplifying the target nucleic acid sequence under isothermal amplification conditions. In some embodiments, said one or more components for amplifying comprise: (i) a forward primer and a reverse primer, wherein the forward primer is capable of hybridizing to a sequence of a first strand of the target nucleic acid sequence, and the reverse primer is capable of hybridizing to a sequence of a second strand of the target nucleic acid sequence; and/or (ii) an enzyme having a hyperthermophile polymerase activity capable of generating a nucleic acid amplification product. In some embodiments, the enzyme having a hyperthermophile polymerase activity has an amino acid sequence that is at least about 90% identical to the amino acid sequence of SEQ ID NO: 1 or a functional fragment thereof. In some embodiments, the enzyme having a hyperthermophile polymerase activity has an amino acid sequence that is at least about 95% identical to the amino acid sequence of SEQ ID NO: 1. In some embodiments, the enzyme having a hyperthermophile polymerase activity is a polymerase comprising the amino acid sequence of SEQ ID NO: 1. In some embodiments, the quality control template, the signal-generating oligonucleotide, the quality control primer, the supplemental quality control primer, and/or the one or more components for amplifying, are in a lyophilized or freeze-dried form and/or are present in the reagent composition.

Disclosed herein include reaction mixtures. In some embodiments, the reaction mixture comprises: a quality control template disclosed herein; a quality control primer disclosed herein; a signal-generating oligonucleotide disclosed herein; a supplemental quality control primer disclosed herein; a target nucleic acid sequence; and/or one or more additional primers and/or one or more probes specific to a target nucleic acid sequence.

In some embodiments, the one or more additional primers and/or one or more probes specific to the target nucleic acid sequence comprise: a forward primer and/or a reverse primer, wherein the forward primer is capable of hybridizing to a sequence of a first strand of the target nucleic acid sequence, and the reverse primer is capable of hybridizing to a sequence of a second strand of the target nucleic acid sequence; and/or a target signal-generating oligonucleotide capable of hybridizing to a nucleic acid amplification product generated by amplifying the target nucleic acid sequence. The reaction mixture can comprise: one or more of an enzyme having a polymerase activity, dNTPs, and a buffering agent. In some embodiments, the enzyme is an enzyme having a hyperthermophile polymerase activity. In some embodiments, the enzyme having a hyperthermophile polymerase activity has an amino acid sequence that is at least about 90% identical to the amino acid sequence of SEQ ID NO: 1 or a functional fragment thereof.

### BRIEF DESCRIPTION OF THE DRAWINGS

FIG. 1 depicts a non-limiting exemplary embodiment of a DNA hairpin internal control assay disclosed herein. IC Primer, Internal Control Primer; HpIC1 MB2, Hairpin Internal Control Molecular Beacon; HpIC1 P1, Hairpin Internal Control Product 1; HpIC1 P2, Hairpin Internal Control Product 2.

FIG. 2 depicts a non-limiting exemplary embodiment of thermal settings for Hairpin IC (HPIC) isothermal amplification reactions described herein.

FIG. 3A-FIG. 3B show data related to target Neisseria gonorrhoeae (Ng) (ROX) detection and Hairpin IC detection (HEX) in clean samples (FIG. 3A) as compared to samples comprising 10% normal urine (FIG. 3B). NTC, no target control; cp, copies. The molecular beacon IC (HPIC) is detected via HEX fluorophore and the target (Ng) is detected via ROX fluorophore.

FIG. 4A-FIG. 4D show data related to target Neisseria gonorrhoeae (Ng) (ROX) detection and Hairpin IC detection (HEX). FIG. 4A shows a Ng only reaction and FIGS. 4B-FIG. 4D depict reactions with increasing levels of IC template (ICT): 0 ICT (FIG. 4B), 50 k ICT (FIG. 4C), and 500 k ICT (FIG. 4D). Star in FIG. 4D indicates that for that sample, bubbles were present in the pipette tip. NTC, no target control; cp, copies. The molecular beacon IC (HPIC) is detected via HEX fluorophore and the target (Ng) is detected via ROX fluorophore.

FIG. 5A-FIG. 5C show data related to target Neisseria gonorrhoeae (Ng) (ROX) detection and Hairpin IC detection (HEX) in samples with increasing levels of inhibitory urine: 10% (FIG. 5A), 20% (FIG. 5B), and 30% (FIG. 5C). NTC, no target control; cp, copies. The molecular beacon IC (HPIC) is detected via HEX fluorophore and the target (Ng) is detected via ROX fluorophore.

FIG. 6A depicts data related to Ng detection in normal urine in the absence of an internal control. Ng ROX probe was used for target detection and Syto 82 intercalating dye was used for amplification product detections. FIG. 6B depicts data related to Ng/Hairpin IC detection in normal urine where the hairpin IC probe (HPIC) is detected via HEX fluorophore and the target (Ng) is detected via ROX fluorophore.

FIG. 7A-FIG. 7B depict data showing a comparison of target Neisseria gonorrhoeae (Ng) (ROX) detection and Hairpin IC detection (HEX) in reactions employing Hairpin IC from different lots: 9.14 (FIG. 7A) and 10.12 (FIG. 7B). NTC, no target control; cp, copies. The molecular beacon IC (HPIC) is detected via HEX fluorophore and the target (Ng) is detected via ROX fluorophore.

FIG. 8A-FIG. 8B show a non-limiting exemplary schematic of an isothermal amplification reaction provided herein.

FIG. 9A-FIG. 9F depict data related to the detection of amplified internal control via molecular beacons HpIC1b MB1 and HpIC1b MB2 (FIG. 9A-FIG. 9C) and intercalating dye (FIG. 9D-FIG. 9F). A hairpin-shaped internal control target was amplified in APA reaction for 10 minutes with simultaneous detection by detection probe (FIG. 9A) and intercalating dye (FIG. 9D). The hairpin-shaped internal control was labeled with HEX at the 5′end and IBFQ at the 3′end. In some such embodiments, it functions as both internal control target and detection probe. After the reaction, the temperature of the reactions was immediately ramped up from assay temperature to 90° C. for melting curve analysis (FIG. 9B and FIG. 9E) and melt derivatives assessment (FIG. 9C and FIG. 9F).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Disclosed herein include methods for monitoring an amplification reaction. In some embodiments, the method comprises providing: a quality control template comprising: a 5′ subdomain; a 3′ subdomain; and a loop domain situated between the 5′ subdomain and the 3′ subdomain, wherein intramolecular nucleotide base pairing between the 5′ subdomain and the 3′ subdomain are capable of forming a paired stem domain. In some embodiments, the method comprises providing: a quality control primer capable of hybridizing to at least a portion of the 3′ subdomain. In some embodiments, the method comprises: subjecting the quality control template and the quality control primer to an amplification reaction capable of generating a first quality control product. In some embodiments, the method comprises: detecting the first quality control product. In some embodiments, the amplification reaction is conducted in an amplification reaction mixture under an amplification condition (e.g., an isothermal amplification condition). In some embodiments, subjecting the quality control template and the quality control primer to an amplification reaction capable of generating a first quality control product comprises: amplifying the quality control template with the quality control primer in the amplification reaction mixture under the amplification condition, thereby generating the first quality control product. In some embodiments, the amplification reaction comprises a reverse transcription reaction.

Disclosed herein include kits for monitoring an amplification reaction. In some embodiments, the kit comprises: a quality control template disclosed herein; a quality control primer disclosed herein; a signal-generating oligonucleotide disclosed herein; and/or a supplemental quality control primer disclosed herein.

Disclosed herein include reaction mixtures. In some embodiments, the reaction mixture comprises: a quality control template disclosed herein; a quality control primer disclosed herein; a signal-generating oligonucleotide disclosed herein; a supplemental quality control primer disclosed herein; a target nucleic acid sequence; and/or one or more additional primers and/or one or more probes specific to a target nucleic acid sequence.

### Hairpin Internal Controls

Provided herein include methods, compositions, kits, and reaction mixtures which can, in some embodiments, use Archaeal Polymerase Amplification (“APA”) to isothermally amplify a region of interest within a target nucleotide template for the purpose of real-time analyte detection while simultaneously monitoring or evaluating the amplification reaction (e.g., an Internal Control (“IC”) assay). The methods, compositions, reaction mixtures, and kits provided herein can overcome the challenges presented by competition of target amplification and address the above-mentioned needs in the art by taking advantage of stability of hairpin structures to reduce non-specific interactions with the primary target amplification. The advantages of the hairpin IC systems, methods, compositions, reaction mixtures, and kits provided herein can include reduced assay complexity and reduced undesirable interactions with target amplification. Unexpectedly, in some embodiments, target amplification, when duplexed with a hairpin IC, shows improvement for low copy detection as compared to a target-only assay.

Disclosed herein are methods, compositions, reaction mixtures, and kits for an internal control assay that can be duplexed with a specific target assay and can report the integrity of core reagents in the reaction, instrument failure, and/or sample inhibition in the absence of specific signals from target amplification. In some embodiments, the internal control assay is designed to leverage APA to simultaneously amplify a DNA target and an internal control template for real-time detection under isothermal conditions.

The disclosed internal control approach can enable the concurrent amplifications of specific target(s) and an internal control (IC) by taking advantage of the characteristic structural stability of stem-loop hairpins. Unlike currently available IC methods wherein a linear DNA is used as IC template, a hairpin-shaped template (e.g., quality control template) is used in some embodiments of the methods and compositions provided herein. Due to the complementary nature of the hairpin stem, only a single primer complementary to the 3′ end of the stem is needed for amplification of the hairpin IC target in some embodiments. The IC primer (e.g., quality control primer) can extend on the hairpin template generating a first-round hairpin product (e.g., a first quality control product) complementary to the IC template. The following reiterative extensions of the IC template driven by the single IC primer can generate two hairpin products with the stem structure and complementary loop sequences. The generated IC products can be detected by a signal-generating oligonucleotide (e.g., a probe, a molecular beacon). In some embodiments, the signal-generating oligonucleotide (e.g., molecular beacon) is modified with LNAs to enhance the detectability of hairpin products. In some embodiments, the hairpin template can be labeled with a signal-generating moiety and functions as both template and detection probe. In some embodiments, there is provided an DNA internal control which includes an IC primer and a hairpin probe acting also as the IC template.

The IC approach described herein can be extended to RNA IC assays comprising an IC primer, a signal-generating oligonucleotide (e.g., molecular beacon) and/or a hairpin-shaped IC template. The IC template can contain an RNA segment at the 5′ end and a hairpin-shaped DNA segment with RT primer sequence at the 3′ end in the stem region. In the presence of reverse transcriptase, the RT primer in the stem region of the hairpin can extend over the RNA segment of the IC template, generating cDNA which is also complementary to the IC primer. The subsequent amplification driven by the single IC primer can generate hairpin-shaped products which are complementary in the loop region which can be detected by a signal-generating oligonucleotide (e.g., molecular beacon). In some embodiments, the signal-generating oligonucleotide (e.g., molecular beacon) is modified with LNAs.

The methods, compositions, reaction mixtures, and kits disclosed herein advantageously employ a hairpin-based IC approach that can permit strong internal control amplification without the risk of competition with target amplification. In some embodiments, only a single IC primer is needed. In some embodiments, a high concentration of IC primer can be used without affecting target amplification. In some embodiments, high IC template copy numbers can be used without hampering target amplification, e.g., used at 50,000 to 500,000 copies, as compared to 20-100 copies of IC target commonly used in other IC systems to reduce competition with target amplification. In some embodiments provided herein wherein a hairpin IC probe is also used as a quality control template, the copy number can be even greater (e.g., in the 25-50 nanomolar concentration range, or >10¹² copies). In some embodiments, only a single primer and a hairpin oligonucleotide labeled with a signal-generating moiety which functions as both template and detection probe are needed. A high concentration of the hairpin oligonucleotide can be used without affecting target amplification. Without being bound by any particular theory, less primer-dimers and false priming can occur due to the use of a single primer and hairpin-shaped template and products.

In some embodiments, the internal control assay comprises a single primer, a hairpin-shaped internal control template, and/or a signal-generating oligonucleotide (e.g., molecular beacon) for detection of amplified hairpin products. In some embodiments, the internal control assay comprises a single primer, and a signal-generating oligonucleotide (e.g., molecular beacon) that functions as both template and detector.

In some embodiments, the signal-generating oligonucleotide (e.g., molecular beacon) comprises a fluorophore at the 5′ end and a quencher at the 3′ end. In some embodiments, the signal-generating oligonucleotide (e.g., molecular beacon) can be replaced by a quencher-free, hairpin-shaped DNA probe to detect hybridization to hairpin amplification products. In the absence of IC amplification, the fluorescence of the hairpin probe with a fluorophore attached at the 5′-end can be quenched by guanine bases and/or adenine bases in the complementary stem. Without being bound by any particular theory, upon hybridization, a conformational reorganization can occur resulting in an increase in fluorescence intensity.

Underlying technical principles used for the designs of hairpin-based internal controls provided herein can include nearest neighbor (NN) theory for prediction of DNA thermodynamics using energy values, and secondary structure dynamics of DNA Hairpins. Without being bound by any particular theory, the mechanism of hairpin amplification and its advantages as an internal control can be related to the thermodynamics of hairpin stem-loop structures.

Additional embodiments of the methods and compositions provided herein include a probe-free version of the hairpin-based internal control. In this approach, a single IC primer labeled with a fluorophore at the 5′ end and a hairpin template can be the only two components necessary for IC amplification and detection. The 5′ end of the primer can contain one or more cytosine bases adjacent to the fluorophore. The labeled primer can copy the hairpin template exponentially, resulting in hairpin products wherein the 5′ end fluorophore is quenched due to the proximity of guanine base(s) via photo-induced electron transfer. In some embodiments, the single IC primer labeled with a fluorophore at the 5′ end can be the only component necessary for IC amplification and detection. The 5′ end of the primer can contain one or more cytosine bases adjacent to the fluorophore. The 3′ end of the labeled primer is self-complementary and can be copied exponentially, resulting in palindrome hairpin products wherein the 5′ end fluorophore is quenched due to the proximity of guanine base(s) via photo-induced electron transfer. In this one component DNA IC assay, the (i) the quality control template, (ii) the quality control primer, and (iii) the signal-generating oligonucleotide, are present in the same molecule.

### Three-Component IC Assays

In some embodiments, the RNA Hairpin IC assay includes an IC primer, a signal-generating oligonucleotide (e.g., molecular beacon) and/or a hairpin shaped IC template. The IC template can comprise an RNA segment at the 5′ end and a hairpin-shaped DNA segment with RT primer sequence at the 3′ end in the stem region. In the presence of reverse transcriptase, the RT primer in the stem region of the hairpin can extend over the RNA segment of the IC template, generating cDNA which is also complementary to the IC primer. Without being bound by any particular theory, the subsequent amplification can be entirely driven by the single IC primer forming pan-handle shaped products. For ease of amplification and detection, the product hybridization melting temperature (Tm) can be designed to be greater than or equal to the product hairpin Tm. A signal-generating oligonucleotide (e.g., molecular beacon) modified with locked nucleic acids (LNAs) in the spacer region can be used for IC product detection in some embodiments. In some embodiments, a fluorescence dye can be used for amplified IC product detection.

The DNA Hairpin IC assay can comprise a hairpin-shaped IC template, a single IC primer and/or a beacon. In some embodiments, the IC primer can extend on the IC template, generating two “pan-handle” shaped products which are complementary in the loop (spacer) region. Without being bound by any particular theory, the following reiterative extensions of IC primer on pan-handle products can generate exponential amplification. For ease of amplification and detection, the product hybridization Tm can be designed to be greater than or equal to product hairpin Tm. A signal-generating oligonucleotide (e.g., molecular beacon) modified with LNAs can be used for IC product detection in some embodiments. In some embodiments, a fluorescence dye can be used for amplified IC product detection.

The disclosed Hairpin IC method can provide, in some embodiments, reduced primer-related interactions with the target being assayed. In some embodiments, only three IC amplification components are needed: a primer, a template and a probe (e.g., a molecular beacon) for either DNA or RNA IC assays. In an RNA IC assay provided herein, the RT primer can be embedded in a chimeric IC template. In some embodiments, the combination of low IC primer concentration and formation of “pan-handle” structures can further reduce nonspecific interactions with the target being assayed.

### Two-Component IC Assays

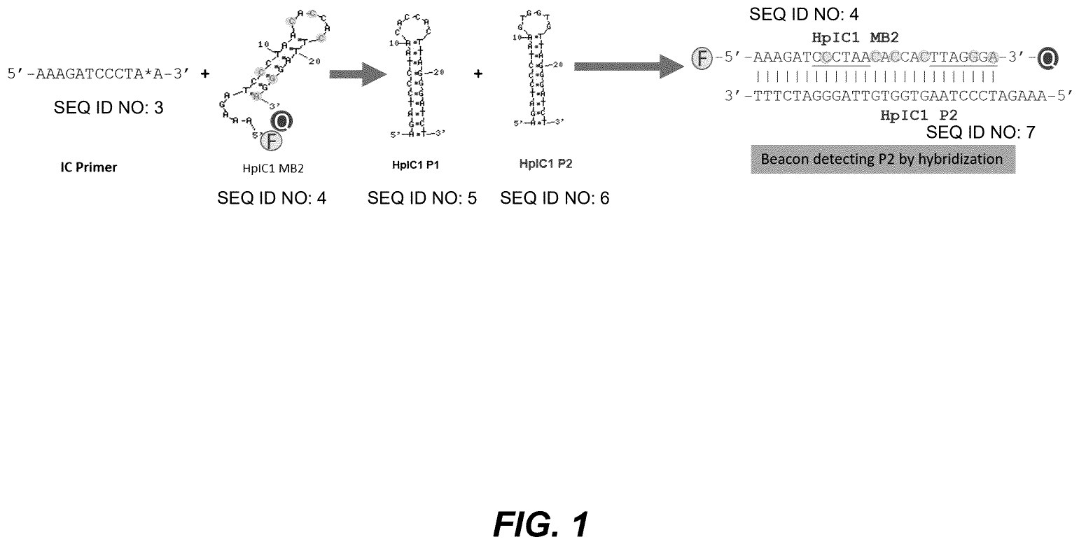

In some embodiments, the hairpin IC method comprises a single IC primer and a signal-generating oligonucleotide (e.g., molecular beacon) for both IC amplification and detection (FIG. 1). The sequence of the signal-generating oligonucleotide (e.g., molecular beacon) can include partial or entire IC primer, the spacer region of the IC template and 4-5 nucleotides adjacent to IC spacer and complementary to the 3′end of the IC primer. The IC primer can hybridize to the beacon and can generate a first-round extension product whose 3′ end is complementary to the IC primer. The subsequent extension of IC primer can generate a “pan-handle” shaped internal control product. Without being bound by any particular theory, the following reiterative extensions of IC primer on pan-handle products can be entirely driven by the single IC primer. The IC product generated can be detected by the IC beacon modified with, e.g., LNAs, to enhance the detectability of pan-handle products.

The two-component hairpin-based IC approaches described herein can be extended to RNA IC assays. The signal-generating oligonucleotide (e.g., molecular beacon) in this case can comprise a few RNA bases in the loop region. An RT primer can extend along the RNA bases in the beacon, generating cDNA. The RNA bases in the loop region can be degraded by the RNase H activity in reverse transcriptase during cDNA synthesis, resulting in fluorescence signal release. Depending on the beacon design, in some embodiments, an IC primer can be employed to improve exponential amplification.

Advantageously, this approach can have the least primer-related interactions with the target being assayed relative to alternative methods of assay monitoring. There are, in some embodiments, only two IC components of a DNA IC assay: a primer and a hairpin probe (e.g., beacon) for both IC amplification and detection as internal control. For two-component RNA IC assays provided herein, an RT primer and a signal-generating oligonucleotide (e.g., molecular beacon) containing an RNA stretch in the loop region can be the minimal components, with an optional IC primer employed in some embodiments.

### Methods of Monitoring Amplification Reactions

Disclosed herein include methods for monitoring an amplification reaction. In some embodiments, the method comprises providing: a quality control template comprising: a 5′ subdomain; a 3′ subdomain; and a loop domain situated between the 5′ subdomain and the 3′ subdomain, wherein intramolecular nucleotide base pairing between the 5′ subdomain and the 3′ subdomain are capable of forming a paired stem domain. In some embodiments, the method comprises providing: a quality control primer capable of hybridizing to at least a portion of the 3′ subdomain. In some embodiments, the method comprises: subjecting the quality control template and the quality control primer to an amplification reaction capable of generating a first quality control product. In some embodiments, the method comprises: detecting the first quality control product. The amplification reaction can be conducted in an amplification reaction mixture under an amplification condition (e.g., an isothermal amplification condition). In some embodiments, subjecting the quality control template and the quality control primer to an amplification reaction capable of generating a first quality control product comprises: amplifying the quality control template with the quality control primer in the amplification reaction mixture under the amplification condition, thereby generating the first quality control product. The amplification reaction can comprise a reverse transcription reaction.

The method can comprise: providing an enzyme having a polymerase activity (e.g., an enzyme having a hyperthermophile polymerase activity). The method can comprise: providing a reverse transcriptase. In some embodiments, the enzyme having a hyperthermophile polymerase activity has a reverse transcriptase activity. The amplification reaction can comprise: contacting the quality control primer with the quality control template for hybridization, and extending the quality control primer hybridized to the quality control template with an enzyme having a polymerase activity, thereby generating a first quality control product. The amplification reaction can comprise: contacting the quality control primer with the first quality control product for hybridization, and extending the quality control primer hybridized to the first quality control product with an enzyme having a polymerase activity, thereby generating a second quality control product. The amplification reaction can comprise: contacting the quality control primer with the second quality control product for hybridization, and extending the quality control primer hybridized to the second quality control product with an enzyme having a polymerase activity, thereby generating a first quality control product. The first quality control product and second quality control product can comprise a 5′ subdomain and the 3′ subdomain capable of forming a paired stem domain. In some embodiments, the first quality control product and second quality control product have the same stem domain. The first quality control product and the second first quality control product can comprise a loop domain complementary to each other. The amplification reaction can comprise linear and/or exponential amplification the first quality control product and the second quality control product. The 5′ subdomain can comprise the sequence of at least a portion of the quality control primer. The first quality control product and the second quality control product can be both capable of forming a hairpin structure. As used herein, the term “hairpin structure” shall be given its ordinary meaning, and shall also refer to a double-helical region formed by base pairing between adjacent, inverted, complementary sequences in a single strand of RNA or DNA. The quality control template can comprise a 5′ terminal domain situated 5′ of the 5′ subdomain, and/or the quality control template can comprise a 3′ terminal domain situated 3′ of the 3′ subdomain. The 5′ terminal domain of the quality control template can comprise at least a portion of the sequence of the quality control primer. The combined sequence of the 5′ terminal domain and the 5′ subdomain can comprise the entire sequence of the quality control primer.

Detecting the first quality control product can comprise detecting the first quality control product with a signal-generating oligonucleotide. The signal-generating oligonucleotide can be capable of hybridizing to the first quality control product. The detecting can comprise contacting the first quality control product with the signal-generating oligonucleotide for hybridization. The signal-generating oligonucleotide can comprise a quencher, a label, or both. The label can comprise a quenchable label (e.g., a fluorophore). The quenchable label can be, for example, a fluorophore. As used herein, the term “fluorophore” shall be given its ordinary meaning and also refers to any reporter group whose presence can be detected by its light emitting properties. Non-limiting examples of fluorophore include: Cy2™ (506), YO-PRO™-1 (509), YOYO™-1 (509), Calcein (517), FITC (518), FluorX™ (519), Alexa™ (520), Rhodamine 110 (520), Oregon Green™ 500 (522), Oregon Green™ 488 (524), RiboGreen™ (525), Rhodamine Green™ (527), Rhodamine 123 (529), Magnesium Green™ (531), Calcium Green™ (533), TO-PRO™-1 (533), TOTO1 (533), JOE (548), BODIPY530/550 (550), Dil (565), BODIPY TMR (568), BODIPY558/568 (568), BODIPY564/570 (570), Cy3™ (570), Alexa™ 546 (570), TRITC (572), Magnesium Orange™ (575), Phycoerythrin R&B (575), Rhodamine Phalloidin (575), Calcium Orange™ (576), Pyronin Y (580), Rhodamine B (580), TAMRA (582), Rhodamine Red™ (590), Cy3.5™ (596), ROX (608), Calcium Crimson™ (615), Alexa™ 594 (615), Texas Red (615), Nile Red (628), YO-PRO™-3 (631), YOYO™-3 (631), R-phycocyanin (642), C-Phycocyanin (648), TO-PRO™-3 (660), TOTO3 (660), DiD DilC (5) (665), Cy5™ (670), Thiadicarbocyanine (671), Cy5.5 (694), HEX (556), TET (536), Biosearch Blue (447), CAL Fluor Gold 540 (544), CAL Fluor Orange 560 (559), CAL Fluor Red 590 (591), CAL Fluor Red 610 (610), CAL Fluor Red 635 (637), FAM (520), Fluorescein (520), Fluorescein-C3 (520), Pulsar 650 (566), Quasar 570 (667), Quasar 670 (705) and Quasar 705 (610) (the numbers in parentheses are maximum emission wavelength in nanometers for the responding fluorophore). In some embodiments, the fluorophore is Cy5™. In some embodiments, the fluorophore is hexachlorofluorescein (HEX).

The signal-generating oligonucleotide can comprise a quencher. The quencher can be capable of quenching the label. Quenching can be mediated by fluorescence resonance energy transfer (FRET). FRET is based on classical dipole-dipole interactions between the transition dipoles of the donor (e.g., a fluorophore) and acceptor (e.g., a quencher) and is dependent on the donor-acceptor distance. FRET can typically occur over distances up to 100 Å. FRET also depends on the donor-acceptor spectral overlap and the relative orientation of the donor and acceptor transition dipole moments. Quenching of a fluorophore can also occur as a result of the formation of a non-fluorescent complex between a fluorophore and another fluorophore or non-fluorescent molecule. This mechanism is known as “contact quenching,” “static quenching,” or “ground-state complex formation.” Without being bound by any particular theory, it is believed that a quencher moiety is not required in some embodiments of the method disclosed herein in order to observe a detectable change in fluorescence, and proximal-base quenching effects are sufficient to produce a detectable shift in fluorescence to allow evaluating, monitoring, observing, and/or tracking a nucleic acid amplification reaction. Examples of the quencher include, but are not limited to, Iowa Black FQ, Iowa Black RQ, Black Hole Quencher-1 (BHQ-1), Black Hole Quencher-2 (BHQ-2), TMR, QSY-7, and Dabcyl.

The detecting can comprise contacting the first quality control product with the signal-generating oligonucleotide for hybridization. The label can be capable of generating a signal upon the signal-generating oligonucleotide hybridizing the first quality control product. In some embodiments, upon the signal-generating oligonucleotide hybridizing the first quality control product, the label generates a signal. The signal can be fluorescence. Detecting the first quality control product can comprise detecting a signal generated by the label of the signal-generating oligonucleotide. The label can be a fluorophore and the signal can be fluorescence. The detecting can comprise detecting the signal of the label before the amplification reaction, during the amplification reaction, after the amplification reaction, or any combination thereof.

The method can comprise: providing a signal-generating oligonucleotide; subjecting the signal-generating oligonucleotide to the amplification reaction; and detecting the first quality control product with the signal-generating oligonucleotide. The quality control template can be a signal-generating oligonucleotide. The quality control template can be (i) a template for the synthesis of the first quality control product, and (ii) a means of detecting the first quality control product. The signal-generating oligonucleotide can be capable of (i) detecting the first quality control product and (ii) being a template for the quality control primer-driven synthesis of the first quality control product.

In some embodiments, the 5′ terminal domain of the quality control template comprises: one or more RNA nucleotides; and/or the sequence of at least a portion of the quality control primer. In some embodiments, the quality control template does not comprise a 3′ terminal domain. The 3′ end of the quality control template can be complementary to the 5′ end of the 5′ subdomain of the quality control template. In some embodiments, a reverse transcriptase can be capable using the one or more RNA nucleotides of the 5′ terminal domain of the quality control template as a template to extend the 3′ end of the quality control template, thereby generating an extended quality control template. The 3′ end of the extended quality control template can comprise a sequence complementary to at least a portion of the quality control primer. The amplification reaction can comprise contacting a reverse transcriptase with the quality control template, thereby generating an extended quality control template. The extended quality control template can comprise cDNA. In some embodiments, the amplification reaction comprises: contacting the quality control primer with the 3′ end of the extended quality control template for hybridization, and extending the quality control primer hybridized to the 3′ end of the extended quality control template with a reverse transcriptase and/or an enzyme having a polymerase activity, thereby generating a first quality control product.

The quality control template can be a signal-generating oligonucleotide. The signal-generating oligonucleotide can comprise a label. The loop domain can comprise one or more RNA nucleotides. The label can comprise a quenchable label (e.g., a fluorophore). The signal-generating oligonucleotide can comprise a quencher. The label can be situated in the 3′ terminal domain and the quencher can be situated in the 5′ terminal domain, and/or the label can be situated in the 5′ terminal domain and the quencher can be situated in the 3′ terminal domain. The amplification reaction can comprise: contacting the quality control primer with the quality control template for hybridization, and extending the quality control primer hybridized to the quality control template with a reverse transcriptase, thereby generating a first quality control product. The reverse transcriptase can comprise RNaseH activity. In some embodiments, the reverse transcriptase cleaves the quality control template at the one or more RNA nucleotides during the generation of the first quality control product, thereby generating a first cleavage product comprising a label and a second cleavage product. Detecting the first quality control product can comprise detecting a signal generated by the first cleavage product comprising a label. Detecting the first quality control product can comprise detecting the signal of a label. The first cleavage product can comprise a label. The amount of signal detected can indicate the absence, presence, or amount of the first cleavage product. The absence, presence, or amount of the first cleavage product can indicate the absence, presence, or amount of the first quality control product. The label can be a fluorophore and the signal can be fluorescence. The method can comprise: providing a supplemental quality control primer; and subjecting the supplemental quality control primer to the amplification reaction.

The signal-generating oligonucleotide can be about 10 nucleotides to about 100 nucleotides in length. The quality control template can be about 10 nucleotides to about 100 nucleotides in length. The quality control primer and/or the supplemental quality control primer can be about 5 nucleotides to about 25 nucleotides in length. The 5′ subdomain, the 3′ subdomain, the loop domain, the 5′ terminal domain, and/or the 3′ terminal domain can be about 1 nucleotide to about 25 nucleotides in length. The signal-generating oligonucleotide, the quality control template, and/or the quality control primer can comprise one or more phosphorothioate linkages and/or one or more locked nucleic acids. The signal-generating oligonucleotide can be a TaqMan detection probe oligonucleotide, a molecular beacon detection probe oligonucleotide, or a molecular torch detection probe oligonucleotide.

The signal-generating oligonucleotide can comprise one or more LNAs. The one or more LNAs can be situated within the loop domain (e.g., the one or more LNAs enhance the detectability of the first quality control product). The signal-generating oligonucleotide can be configured such that the melting temperature (Tm) of first quality control product/signal-generating oligonucleotide duplex is equal to or greater than the melting temperature (Tm) of the paired stem domain of the signal-generating oligonucleotide (e.g., configured via one or more modifications and/or modified bases, such as LNAs situated in the loop domain). LNAs can be situated in the stem domain. In some embodiments, LNAs are also placed in the stem region to strengthen the hairpin stability and enhance the detectability. In addition, and without being bound by any particular theory, in some embodiments when archaeal polymerases are used, the 3′ terminal base is LNA modified (or with other modifications) to block the 3′ to 5′ exonuclease activity of the polymerases. The stem LNAs can be most effective in configuring the Tm of the probe to be above the assay temperature, and thereby (i) stabilizing the hairpin and reducing baseline signals and/or (ii) enhancing the Tm of the quality control product/signal-generating oligonucleotide duplex, thus enhancing the detectability of quality control product. In some embodiments, and without being bound by any particular theory, there is difference between LNAs in 5′ Stem and 3′ stem. In some embodiments, when situated in the stem at the 3′ end, the LNAs enhance the priming of the IC primer and increase the Tm of quality product/signal-generating oligonucleotide duplex. In some embodiments, when situated at the 5′ stem, the LNAs can slow or inhibit the IC product generation even though they can enhance the Tm of the quality control product/signal-generating oligonucleotide duplex. Modifications and modified bases can include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S—S, dithiol, thiol modifier C6 S—S)); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxyInosine, Super T®, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, phosphorothioate linkages, 3′ phosphate groups, spacer bases (e.g., C3 spacer or other spacer bases). The one or more modified nucleotides can comprise a spacer, an a-basic site, an un-methylated RNA base, a 2′-O-methylated nucleotide, and any combination thereof.

In some embodiments, the signal-generating oligonucleotide does not comprise a dye capable of quenching the label. In some embodiments, the signal-generating oligonucleotide does not comprise a moiety capable of quenching the label other than the nucleotides of said signal-generating oligonucleotide. The 5′ terminal domain of the quality control template and/or the signal-generating oligonucleotide can comprise the label. The 5′ terminal domain and/or the 5′ subdomain of the quality control template and/or the signal-generating oligonucleotide can comprise one or more cytosine bases. The 3′ terminal domain and/or the 3′ subdomain of the quality control template and/or the signal-generating oligonucleotide can comprise one or more guanine bases and/or adenine bases. The one or more guanine bases and/or adenine bases can be capable of quenching the label upon the quality control template and/or the signal-generating oligonucleotide forming a hairpin structure. The 3′ terminal domain of the quality control template and/or the signal-generating oligonucleotide can comprise the label. The 3′ terminal domain and/or the 3′ subdomain of the quality control template and/or the signal-generating oligonucleotide can comprise one or more cytosine bases. The 5′ terminal domain and/or the 5′ subdomain of the quality control template and/or the signal-generating oligonucleotide can comprise one or more guanine bases and/or adenine bases. The one or more guanine bases and/or adenine bases can be capable of quenching the label upon the quality control template and/or the signal-generating oligonucleotide forming a hairpin structure.

Detecting the first quality control product can comprise detecting a reduction in the amount of signal generated by a label of the quality control primer. The label can be a fluorophore and the signal can be fluorescence. The generation of the first quality control product and second quality control product can be correlated with a decline in the signal detected. The 5′ end of the quality control primer can comprise a label, and the quality control primer can comprise one or more cytosine bases adjacent to the label. The 3′ terminal domain and/or the 3′ subdomain of the quality control template, first quality control product, and/or the second quality control product can comprise one or more guanine bases and/or adenine bases. Upon the quality control primer binding the quality control template and/or second quality control product and being extended with an enzyme having a polymerase activity to generate a first quality control product, the one or more guanine bases and/or adenine bases present in the 3′ terminal domain and/or the 3′ subdomain of the first quality control product can be capable of quenching the label upon first quality control product forming a hairpin structure. Upon the quality control primer binding the first quality control product and being extended with an enzyme having a polymerase activity to generate a second quality control product, the one or more guanine bases and/or adenine bases present in the 3′ terminal domain and/or the 3′ subdomain of the second quality control product can be capable of quenching the label upon second quality control product forming a hairpin structure. Detecting the first quality control product can comprise contacting the first quality control product with a fluorescence dye.

Providing the quality control primer, the quality control template, and/or the signal-generating oligonucleotide can comprise providing a reagent composition comprising the quality control primer, the quality control template, and/or the signal-generating oligonucleotide. Subjecting the quality control primer, the quality control template, and/or the signal-generating oligonucleotide to an amplification reaction can comprise contacting the reagent composition with a treated sample to generate the amplification reaction mixture. The method can comprise detecting a target nucleic acid sequence in a sample. The method can comprise: subjecting the target nucleic acid sequence to an amplification reaction capable of generating a nucleic acid amplification product. The method can comprise: detecting the nucleic acid amplification product with a target signal-generating oligonucleotide, wherein the target signal-generating oligonucleotide is capable of hybridizing to the nucleic acid amplification product. Subjecting the target nucleic acid sequence to an amplification reaction capable of generating a nucleic acid amplification product can comprise amplifying the target nucleic acid sequence in the amplification reaction mixture under the amplification condition, thereby generating a nucleic acid amplification product. The method can comprise: contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence. The method can comprise: contacting the reagent composition with the treated sample to generate the amplification reaction mixture, wherein the reagent composition comprises one or more amplification reagents.

The one or more amplification reagents can comprise: a reverse transcriptase; an enzyme having a hyperthermophile polymerase activity (e.g., an enzyme having a hyperthermophile polymerase activity and a reverse transcriptase activity); a forward primer; a reverse primer; a reverse transcription primer; and/or dNTPS. The sample nucleic acids can comprise a nucleic acid comprising the target nucleic acid sequence. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and ii) an enzyme having a hyperthermophile polymerase activity, thereby generating the nucleic acid amplification product. In some embodiments, the nucleic acid is: a dsDNA; and/or a product of reverse transcription reaction. The nucleic acid can be a product of reverse transcription reaction generated from sample ribonucleic acids (e.g., the amplifying comprises generating the nucleic acid by a reverse transcription reaction). The amplification reaction can be performed for a period of about 5 minutes to about 60 minutes. In some embodiments, amplifying the quality control template can comprise generating the first quality control product and/or the second quality control product at detectable levels within about 20 minutes, about 15 minutes, or about 10 minutes.

In some embodiments, the method: is performed in a single reaction vessel; does not comprise using any enzymes other than the reverse transcriptase and the enzyme having a hyperthermophile polymerase activity; does not comprise using any enzyme other than the enzyme having a hyperthermophile polymerase activity; does not comprise heat denaturing and/or enzymatic denaturing the nucleic acid and/or quality control template during the amplification step; and/or does not comprise contacting the nucleic acid and/or quality control template with a signal-stranded DNA binding protein.

In some embodiments, the method, the reagent composition, and/or the amplification reaction mixture does not comprise: a template capable of generating the first quality control product other than the quality control template; a probe capable of detecting of the first quality control product other than the signal-generating oligonucleotide; a double-stranded template capable of generating the first quality control product; a linear template capable of generating the first quality control product; and/or a primer capable of hybridizing to the quality control template, the first quality control product and/or the second quality control product other than the quality control primer.

The method can comprise determining the presence, absence and/or amount of the first quality control product. The presence, absence and/or amount of the signal can indicate the presence, absence and/or amount of the first quality control product. The presence, absence and/or amount of the signal can indicate the presence, absence and/or amount of one or more interfering components in the amplification reaction mixture. The presence, absence and/or amount of the signal can indicate: (i) the integrity of the one or more amplification reagents in the amplification reaction mixture; (ii) failure of the instrument wherein the amplification reaction is conducted; and/or (iii) sample-derived inhibition (e.g., matrix-derived inhibition) of the amplification reaction. The presence, absence and/or amount of the signal can indicate the degree to which the amplification of the target nucleic acid sequence is inhibited in the amplification reaction.

The method, the reagent composition, and/or the amplification reaction mixture can comprise at least about 50,000 copies to about 500,000 copies of the quality control template (e.g., in a 3-component assay). The method, the reagent composition, and/or the amplification reaction mixture can comprise a concentration of IC template/probe of about 25 nM to 50 nM, which can be about 10¹² copies of IC template (e.g., in a 2-component assay). A comparable method of monitoring an amplification reaction can employ about 20 copies to about 100 copies of an internal control template. In some embodiments, the method, the reagent composition, and/or the amplification reaction mixture comprises an at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) greater number of copies of the quality control template and/or quality control primer as compared to a comparable method of monitoring an amplification reaction that does not comprise the quality control template and/or the quality control primer. In some embodiments, said comparable method comprises an internal control template that is not capable of forming a hairpin structure. In some embodiments, a comparable method of monitoring an amplification reaction that does not comprise the quality control template and/or the quality control primer inhibits the amplification of the target nucleic acid sequence and/or the detection of nucleic acid amplification product by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) more than a method disclosed herein. In some embodiments, the quality control template and/or the quality control primer is not capable of hybridizing to the target nucleic acid sequence. In some embodiments, the presence of the quality control template and/or the quality control primer does not inhibit the amplification of the target nucleic acid sequence and/or the detection of nucleic acid amplification product. The presence of the quality control template and/or the quality control primer in the amplification reaction mixture can improve the amplification of the target nucleic acid sequence and/or detection of the nucleic acid amplification product by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a comparable method wherein the quality control template and/or the quality control primer is absent from the amplification reaction mixture. The number of false priming events and/or the generation of primer-dimers can be reduced by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a comparable method of monitoring an amplification reaction that does not comprise the quality control template and/or the quality control primer.

Disclosed herein include reaction mixtures. In some embodiments, the reaction mixture comprises: a quality control template disclosed herein; a quality control primer disclosed herein; a signal-generating oligonucleotide disclosed herein; a supplemental quality control primer disclosed herein; a target nucleic acid sequence; and/or one or more additional primers and/or one or more probes specific to a target nucleic acid sequence. The reaction mixture can comprise one or more of an enzyme having a polymerase activity, dNTPs, and a buffering agent.

Amplifying the target nucleic acid sequence can comprise generating the nucleic acid amplification product and/or quality control product at detectable levels within about 20 minutes, about 15 minutes, or about 10 minutes. The detecting can be performed in less than about 20 minutes, less than about 15 minutes, less than about 10 minutes, or less than about 5 minutes, from the time the reagent composition is contacted with the treated sample.

The lysis buffer can comprise one or more of magnesium sulfate, ammonium sulfate, EDTA, and EGTA. The pH of the lysis buffer can be about 1.0 to about 10.0 (e.g., about 2.2). The sample nucleic acids can comprise sample ribonucleic acids and/or sample deoxyribonucleic acids. The sample nucleic acids can comprise cellular RNA, mRNA, microRNA, bacterial RNA, viral RNA, or a combination thereof. In some embodiments, the one or more amplification reagents comprise: a reverse transcriptase; an enzyme having a hyperthermophile polymerase activity; and/or dNTPS. In some embodiments, the enzyme having a hyperthermophile polymerase activity has a reverse transcriptase activity a forward primer; a reverse primer; a reverse transcription primer. The reagent composition can be lyophilized, heat-dried, and/or comprises one or more additives. In some embodiments, the one or more additives comprise: Tween 20, Triton X-100, and/or tween 80; an amino acid; a sugar or sugar alcohol; and/or a polymer. The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof Contacting the reagent composition with the treated sample can comprise dissolving the reagent composition in the treated sample. In some embodiments, the one or more lytic reagents comprise: about 0.001% (w/v) to about 1.0 (w/v) of the treated sample (e.g., about 0.2% (w/v) of the treated sample); and/or a detergent (e.g., one or more of a cationic surfactant, an anionic surfactant, a non-ionic surfactant, and an amphoteric surfactant).

In some embodiments, the method: is performed in a single reaction vessel; does not comprise using any enzymes other than the reverse transcriptase and the enzyme having a hyperthermophile polymerase activity; does not comprise using any enzyme other than the enzyme having a hyperthermophile polymerase activity; does not comprise heat denaturing and/or enzymatic denaturing the nucleic acid during the amplification step; and/or does not comprise contacting the nucleic acid with a single-stranded DNA binding protein.

The target nucleic acid sequence can comprise a length of no longer than about 20 nucleotides to no longer than about 90 nucleotides (e.g., about 30 nucleotides). The forward primer, the reverse primer, and/or the reverse transcription primer can be about 8 to 16 bases long. The nucleic acid amplification product can be about 20 to 40 bases long. The spacer sequence can comprise a portion of the target nucleic acid sequence. The spacer sequence can be 1 to 10 bases long. The isothermal amplification condition can comprise a constant temperature of about 30° C. to about 72° C., optionally about 55° C. to about 75° C., optionally about 56° C. to about 67° C. The amplifying can be performed: for a period of about 5 minutes to about 60 minutes (e.g., a period of about 15 minutes). The amplifying can be performed: in helicase-free, single-stranded binding protein-free, cleavage agent-free, and recombinase-free, isothermal amplification conditions. The amplifying can be carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). The PCR can be real-time PCR and/or quantitative real-time PCR (QRT-PCR).

The enzyme having a hyperthermophile polymerase activity has an amino acid sequence that can be at least about 90% identical to the amino acid sequence of SEQ ID NO: 1 or a functional fragment thereof. The enzyme having a hyperthermophile polymerase activity has an amino acid sequence that can be at least about 95% identical to the amino acid sequence of SEQ ID NO: 1. The enzyme having a hyperthermophile polymerase activity can be a polymerase comprising the amino acid sequence of SEQ ID NO: 1. In some embodiments, the enzyme having a hyperthermophile polymerase activity has low or no exonuclease activity. The sample ribonucleic acids can be contacted with the reverse transcriptase and the enzyme having a hyperthermophile polymerase activity simultaneously. The sample ribonucleic acids can be contacted with the reverse transcriptase, the enzyme having a hyperthermophile polymerase activity, and the forward and reverse primers simultaneously. The sample ribonucleic acids can be contacted with the reverse transcriptase, the enzyme having a hyperthermophile polymerase activity, the forward primer, the reverse primer, and the reverse transcription primer simultaneously.

There are provided, in some embodiments, quality control primers. The quality control primer can comprise a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 3 or SEQ ID NO: 9. The quality control primer can comprise a sequence that has 1, 2, 3, 4 or more mismatches or universal nucleotides relative to SEQ ID NO: 3 or SEQ ID NO: 9. There are provided, in some embodiments, quality control templates (e.g., Hairpin Internal Control (HPIC) Molecular Beacons). The quality control template can comprise a sequence that is at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) identical to SEQ ID NO: 4, SEQ ID NO: 8, or SEQ ID NO: 11. The quality control template can comprise a sequence that has 1, 2, 3, 4 or more mismatches or universal nucleotides relative to SEQ ID NO: 4, SEQ ID NO: 8, or SEQ ID NO: 11. The quality control primer and quality control template can comprise one or more modifications (e.g., phosphorothioated DNA bases, LNAs). The quality control template can comprise a 5′ modification (e.g., 5HEX) and/or a 3′ modification (e.g., 3IABkFQ). The IC assay components provided herein can be employed in multiplex assays (e.g., in concert with assay detecting C. trachomatis and/or N. Gonorrhea).

In some embodiments, the amplifying comprises and/or does not comprise one or more of the following amplification methods: APA, LAMP, HDA, RPA, SDA, NASBA, TMA, NEAR, RCA, MDA, RAM, cHDA, SPIA, SMART, 3SR, GEAR and IMDA. In some embodiments, the amplifying does not comprise LAMP.

In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

The term “isothermal amplification reaction” shall be given its ordinary meaning and shall also include reactions wherein the temperature does not significantly change during the reaction. In some embodiments, the temperature of the isothermal amplification reaction does not deviate by more than 10° C., for example by not more than 5° C. or by not more than 2° C. during the main enzymatic reaction step where amplification takes place. Depending on the method of isothermal amplification of nucleic acids, different enzymes can be used for amplification. Isothermal amplification compositions and methods are described in PCT Application published as WO2017176404, the content of which is incorporated herein by reference in its entirety.

In some embodiments, the methods and components described herein comprise a storage-stable lysis buffer. In some embodiments, the lysis buffer is resistant to the formation of a precipitate for a period of time under a storage condition (e.g., storage-stable lysis buffer). Compositions, kits, and methods wherein lysis buffers resist precipitation are described in the International Application No. PCT/US23/61980 entitled “NON-OPAQUE LYTIC BUFFER COMPOSITION FORMULATIONS” and filed on Feb. 3, 2023, the content of which is incorporated herein by reference in its entirety.

Some embodiments of the methods and compositions provided herein do not comprise agents and/or conditions that denature nucleic acids (e.g., promote strand separation and/or promote unwinding) other than acids and/or low pH conditions. Compositions, kits, and methods for nucleic acid detection wherein nucleic acid strands are dissociated under low pH conditions (e.g., via contact with an acidic lysis buffer) to facilitate subsequent rapid amplification and detection are described in the International Application No. PCT/US23/61978 entitled “METHOD FOR SEPARATING GENOMIC DNA FOR AMPLIFICATION OF SHORT NUCLEIC ACID TARGETS” and filed on Feb. 3, 2023, the content of which is incorporated herein by reference in its entirety.

In some embodiments, the methods and compositions described herein can comprise a lysis buffer and/or a reagent composition. Lysis buffers comprising a lytic agent and a reducing agent, and reagent compositions comprising amplification agents and one or more protectants (e.g., cyclodextrin compounds) capable of sequestering lytic agents, are described in the International Application No. PCT/US22/21015 entitled “ISOTHERMAL AMPLIFICATION OF PATHOGENS” and filed on Mar. 18, 2022, the content of which is incorporated herein by reference in its entirety.

In some embodiments, the methods and compositions described herein can comprise a signal-generating oligonucleotide comprising one or more polymerase stoppers (e.g., a protected signal-generating oligonucleotide). Compositions, kits, and methods for nucleic acid detection wherein protected signal-generating oligonucleotides enable reduced non-specific product formation and/or fewer false positives are described in the U.S. Provisional Patent Application No. 63/374,772 entitled “MODIFIED MOLECULAR BEACONS FOR IMPROVED DETECTION SPECIFICITY” and filed on Sep. 7, 2022, the content of which is incorporated herein by reference in its entirety.

Some embodiments of the methods and compositions described herein can comprise probe(s) melting at temperatures different than the optimal APA reaction temperature to enable multiplexing targets and/or an internal control(s). Compositions, kits, and methods for multiplexed nucleic acid detection are described in the U.S. Provisional Patent Application No. 63/374,831 entitled “ARCHEAL POLYMERASE AMPLIFICATION” and filed on Sep. 7, 2022, the content of which is incorporated herein by reference in its entirety.

Some embodiments of the methods and compositions described herein can, in some embodiments, be employed in concert with the systems, methods, compositions, and kits for detecting pathogens described in the U.S. Provisional Patent Application No. 63/374,774 entitled “METHODS AND COMPOSITIONS FOR PATHOGEN DETECTION” and filed on Sep. 7, 2022, the content of which is incorporated herein by reference in its entirety.”

### Nucleic Acid, Subjects, Samples and Nucleic Acid Processing

Provided herein are methods and compositions for amplifying nucleic acid. The terms “nucleic acid” and “nucleic acid molecule” may be used interchangeably herein. The terms refer to nucleic acids of any composition, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus, a mitochondria, or cytoplasm of a cell. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid may be used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame, “forward” strand or “reverse” strand) and double-stranded polynucleotides. The term “gene” means the segment of DNA involved in producing a polypeptide chain; and generally includes regions preceding and following the coding region (leader and trailer) involved in the transcription/translation of the gene product and the regulation of the transcription/translation, as well as intervening sequences (introns) between individual coding segments (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

In some embodiments of the methods provided herein, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence may refer to either the sense or antisense strand of a nucleic acid sequence, and also may refer to sequences as they exist on target nucleic acids, amplified copies, or amplification products, of the original target sequence. A target sequence can be a subsequence within a larger polynucleotide. For example, a target sequence can be a short sequence (e.g., 20 to 50 bases) within a nucleic acid fragment, a chromosome, a plasmid, that is targeted for amplification. In some embodiments, a target sequence may refer to a sequence in a target nucleic acid that is complementary to an oligonucleotide (e.g., primer) used for amplifying a nucleic acid. Thus, a target sequence may refer to the entire sequence targeted for amplification or may refer to a subsequence in the target nucleic acid where an oligonucleotide binds. An amplification product may be a larger molecule that comprises the target sequence, as well as at least one other sequence, or other nucleotides. The amplification product can be about the same length as the target sequence, for example exactly the same length as the target sequence. The amplification product can comprise, or consist of, the target sequence.

The length of the target sequence, and/or the guanine cytosine (GC) concentration (percent), may depend, in part, on the temperature at which an amplification reaction is run, and this temperature may depend, in part, on the stability of the polymerase(s) used in the reaction. Sample assays may be performed to determine an appropriate target sequence length and GC concentration for a set of reaction conditions. For example, where a polymerase is stable up to 60° C. to 65° C., then the target sequence may be, for example, from 19 to 50 nucleotides in length, or for example, from about 40 to 50, 20 to 45, 20 to 40, or 20 to 30 nucleotides in length. GC concentration under these conditions may be, for example, less than 60%, less than 55%, less than 50%, or less than 45%.

Target nucleic acid can include, for example, genomic nucleic acid, plasmid nucleic acid, mitochondrial nucleic acid, cellular nucleic acid, extracellular nucleic acid, bacterial nucleic acid and viral nucleic acid. In some embodiments, target nucleic acid may include genomic DNA, chromosomal DNA, plasmid DNA, mitochondrial DNA, a gene, any type of cellular RNA, messenger RNA, bacterial RNA, viral RNA or a synthetic oligonucleotide. Genomic nucleic acid can include any nucleic acid from any genome, for example, animal, plant, insect, viral and bacterial genomes (e.g., genomes present in spores). In some embodiments, genomic target nucleic acid is within a particular genomic locus or a plurality of genomic loci. A genomic locus can include any or a combination of open reading frame DNA, non-transcribed DNA, intronic sequences, extronic sequences, promoter sequences, enhancer sequences, flanking sequences, or any sequences considered associated with a given genomic locus.

The target sequence can comprise one or more repetitive elements (e.g., multiple repeat sequences, inverted repeat sequences, palindromic sequences, tandem repeats, microsatellites, minisatellites, and the like). In some embodiments, a target sequence is present within a sample nucleic acid (e.g., within a nucleic acid fragment, a chromosome, a genome, a plasmid) as a repetitive element (e.g., a multiple repeat sequence, an inverted repeat sequence, a palindromic sequence, a tandem repeat, a microsatellite repeat, a minisatellite repeat and the like). For example, a target sequence may occur multiple times as a repetitive element and one, some, or all occurrences of the target sequence within a repetitive element may be amplified (e.g., using a single pair of primers) using methods described herein. In some embodiments, a target sequence is present within a sample nucleic acid (e.g., within a nucleic acid fragment, a chromosome, a genome, a plasmid) as a duplication and/or a paralog.

Target nucleic acid can include microRNAs. MicroRNAs, miRNAs, or small temporal RNAs (stRNAs) are short (e.g., about 21 to 23 nucleotides long) and single-stranded RNA sequences involved in gene regulation. MicroRNAs may interfere with translation of messenger RNAs and are partially complementary to messenger RNAs. Target nucleic acid can include microRNA precursors such as primary transcript (pri-miRNA) and pre-miRNA stem-loop-structured RNA that is further processed into miRNA. Target nucleic acid can include short interfering RNAs (siRNAs), which are short (e.g., about 20 to 25 nucleotides long) and at least partially double-stranded RNA molecules involved in RNA interference (e.g., down-regulation of viral replication or gene expression).

Nucleic acid utilized in methods described herein can be obtained from any suitable biological specimen or sample, e.g., isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), serum, plasma, urine, aspirate, biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation.

A sample can include samples containing spores, viruses, cells, nucleic acids from prokaryotes or eukaryotes, and/or any free nucleic acid. For example, a method described herein can be used for detecting nucleic acid on the outside of spores (e.g., without the need for lysis). A sample can be isolated from any material suspected of containing a target sequence, such as from a subject described above. In some embodiments, a target sequence is present in air, plant, soil, or other materials suspected of containing biological organisms.

Nucleic acid can be derived (e.g., isolated, extracted, purified) from one or more sources by methods known in the art. Any suitable method can be used for isolating, extracting and/or purifying nucleic acid from a biological sample, including methods of DNA preparation in the art, and various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), GFX™ Genomic Blood DNA Purification Kit (Amersham, Piscataway, N.J.), and the like or combinations thereof. U.S. Pat. No. 7,888,006 provides DNA purification methods and does not disclose the compositions (e.g., lysis buffers, protectants) and methods provided herein

In some embodiments, a cell lysis procedure is performed. Cell lysis can be performed prior to initiation of an amplification reaction described herein (e.g., to release DNA and/or RNA from cells for amplification). Cell lysis procedures and reagents are known in the art and may be performed by chemical (e.g., detergent, hypotonic solutions, enzymatic procedures, and the like, or combination thereof), physical (e.g., French press, sonication, and the like), or electrolytic lysis methods. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl). Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5, for example. In some embodiments, a cell lysis buffer is used in conjunction with the methods and components described herein.

Nucleic acid can be provided for conducting methods described herein without processing of the sample(s) containing the nucleic acid. For example, nucleic acid can be provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components.

Nucleic acid may be provided for conducting methods described herein without modifying the nucleic acid. Modifications can include, for example, denaturation, digestion, nicking, unwinding, incorporation and/or ligation of heterogeneous sequences, addition of epigenetic modifications, addition of labels (e.g., radiolabels such as³²p³³p ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, fluorochromes), and the like. Accordingly, in some embodiments, an unmodified nucleic acid is amplified.

Methods disclosed herein for detecting a target nucleic acid sequence (single-stranded or ds DNA and/or RNA) in a sample can detect a target nucleic acid sequence (e.g., DNA or RNA) with a high degree of sensitivity. In some embodiments, the method can be used to detect a target DNA/RNA present in a sample comprising a plurality of RNAs/DNAs (including the target RNA/DNA and a plurality of non-target RNAs/DNAs), wherein the target RNA/DNA is present at one or more copies per 10, 20, 25, 50, 100, 500, 10³, 5×10³, 10⁴, 5×10⁴, 10⁵, 5×10⁵, 10⁶, or 10⁷, non-target DNAs/RNAs. As used herein, the terms “RNA/DNA” and “RNAs/DNAs” shall be given their ordinary meaning, and shall also refer to DNA, or RNA, or a combination of DNA and RNA.

The threshold of detection, for a method of detecting a target RNA/DNA in a sample, can be, for example 10 nM or less. The term “threshold of detection” shall be given its ordinary meaning, and shall also describe the minimal amount of target RNA/DNA that must be present in a sample in order for detection to occur. As an illustrative example, when a threshold of detection is 10 nM, then a signal can be detected when a target RNA/DNA is present in the sample at a concentration of 10 nM or more. In some embodiments, a disclosed method has a threshold of detection of 5 nM or less, 1 nM or less, 0.5 nM or less, 0.1 nM or less, 0.05 nM or less, 0.01 nM or less, 0.005 nM or less, 0.001 nM or less, 0.0005 nM or less, 0.0001 nM or less, 0.00005 nM or less, 0.00001 nM or less, 10 pM or less, 1 pM or less, 500 fM or less, 250 fM or less, 100 fM or less, 50 fM or less, 500 aM (attomolar) or less, 250 aM or less, 100 aM or less, 50 aM or less, 10 aM or less, or 1 aM or less. In some embodiments, a disclosed composition or method exhibits an attomolar (aM), femtomolar (fM), picomolar (pM), and/or nanomolar (nM), sensitivity of detection.

A sample can comprise sample nucleic acids (e.g., a plurality of sample nucleic acids). The term “plurality” is used herein to mean two or more. Thus, in some embodiments, a sample includes two or more (e.g., 3 or more, 5 or more, 10 or more, 20 or more, 50 or more, 100 or more, 500 or more, 1,000 or more, or 5,000 or more) sample nucleic acids (e.g., DNAs/RNAs). A disclosed method can be used as a very sensitive way to detect a target nucleic acid present in a sample (e.g., in a complex mixture of nucleic acids such as DNAs/RNAs). In some embodiments the sample includes 5, 10, 20, 25, 50, 100, 500, 10³, 5×10³, 10⁴, 5×10⁴, 10⁵, 5×10⁵, 10⁶, or 10⁷, 50, or more, DNAs/RNAs that differ from one another in sequence. In some embodiments, the sample includes DNAs/RNAs from a cell (e.g., a eukaryotic cell, a mammalian cell, or a human cell) or a cell lysate (e.g., a eukaryotic cell lysate, a mammalian cell lysate, a human cell lysate, a prokaryotic cell lysate, a plant cell lysate, or the like).

The term “sample” is used here shall be given its ordinary meaning and shall include any sample that includes RNA and/or DNA (e.g., in order to determine whether a target DNA and/or target RNA is present among a population of RNAs and/or DNAs). The sample can be a biological sample or an environmental sample. The sample can be derived from any source, e.g., the sample can be a synthetic combination of purified DNAs and/or RNAs; the sample can be a cell lysate, an DNA/RNA-enriched cell lysate, or DNAs/RNAs isolated and/or purified from a cell lysate. The sample can be from a patient (e.g., for the purpose of diagnosis). The sample can be from permeabilized cells, crosslinked cells, tissue sections, or combination thereof. The sample can be from tissues prepared by crosslinking followed by delipidation and adjustment to make a uniform refractive index. A sample can include a target nucleic acid (e.g., target DNA/RNA) and a plurality of non-target DNAs/RNAs. In some embodiments, the target DNA/RNA is present in the sample at one copy per 10, 20, 25, 50, 100, 500, 10³, 5×10³, 10⁴, 5×10⁴, 10⁵, 5×10⁵, 10⁶, or 10⁷, non-target DNAs/RNAs.

A sample with respect to a patient encompasses blood and other liquid samples of biological origin, solid tissue samples such as a biopsy specimen or tissue cultures or cells derived therefrom and the progeny thereof, as well as samples that have been manipulated in any way after their procurement (such as by treatment with reagents); washed; or enriched for certain cell populations (e.g., cancer cells) or particular types of molecules (e.g., RNAs). A sample can comprise, or be, a biological sample including but not limited to a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A biological sample can comprise biological fluids derived therefrom (e.g., cancerous cell, infected cell, etc.), e.g., a sample comprising RNAs that is obtained from such cells (e.g., a cell lysate or other cell extract comprising RNAs). In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof.

The source of the sample can be a (or is suspected of being a) diseased cell, fluid, tissue, or organ; or a normal (non-diseased) cell, fluid, tissue, or organ. In some embodiments, the source of the sample is a (or is suspected of being a) pathogen-infected cell, tissue, or organ. For example, the source of a sample can be an individual who may or may not be infected—and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual, as well as cultures thereof. The sample can be a cell-free liquid sample or a liquid sample that comprise cells. Pathogens can be viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis C virus; Hepatitis A virus; Hepatitis B virus; papillomavirus; and the like. Pathogenic viruses can include DNA viruses such as: a papovavirus (e.g., HPV, polyomavirus); a hepadnavirus; a herpesvirus (e.g., HSV (e.g., HSV I, HSV II), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Non-limiting examples of pathogens include Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, human serum parvo-like virus, respiratory syncytial virus, measles virus, adenovirus, human T-cell leukemia viruses, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria sp. (e.g., tenella), Onchocerca volvulus, Leishmania sp., (e.g., tropica), Streptococcus pneumonia, Pneumocystis carinii, Trichophyton rubrum, Entamoeba histolytica, Babesia microti, Giardia lamblia, Cyclospora sp., SARS-CoV-2, Human Immunodeficiency Virus Type 1 (HIV-1), Human T-Cell Lymphotrophic Virus Type 1 (HTLV-1), Herpes Simplex, Herpesvirus 6, Herpesvirus 7, JC Virus, Influenza A, Influenza B, Influenza C, Rotavirus, Human Adenovirus, Human Enteroviruses, Hantavirus, Legionella dumoffii, Mycoplasma fermentans, Haemophilus influenzae, Rickettsia rickettsii, Ehrlichia sp. (e.g., chaffeensis), Borrelia burgdorferi, Yersinia pestis, Chlamydia pneumoniae, Trichinella spiralis, Theileria parva, Taenia hydatigena, Taenia ovis, Taenia saginata, Echinococcus granulosus, Mesocestoides corti, Mycoplasma sp. (e.g., arthritidis), M. hyorhinis, M. orale, M. arginini, Acholeplasma laidlawii, M. salivarium and M. pneumoniae.

### Amplification

Provided herein are methods for amplifying nucleic acid. In some embodiments, nucleic acids are amplified using a suitable amplification process. Nucleic acid amplification typically involves enzymatic synthesis of nucleic acid amplicons (copies), which contain a sequence complementary to a nucleotide sequence being amplified. In some embodiments, an amplification method is performed in a single vessel, a single chamber, and/or a single volume (i.e., contiguous volume). In some embodiments, an amplification method and a detection method (e.g., a detection method described herein) are performed in a single vessel, a single chamber, and/or a single volume (i.e., contiguous volume).

The terms “amplify”, “amplification”, “amplification reaction”, or “amplifying” refer to any in vitro process for multiplying the copies of a target nucleic acid. Amplification sometimes refers to an “exponential” increase in target nucleic acid. “Amplifying” can also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

A generalized description of an amplification process is presented herein. Primers (e.g., oligonucleotides described herein) and target nucleic acid are contacted, and complementary sequences anneal or hybridize to one another, for example. Primers can anneal to a target nucleic acid, at or near (e.g., adjacent to, abutting, and the like) a sequence of interest. A primer annealed to a target may be referred to as a primer-target hybrid, hybridized primer-target, or a primer-target duplex. The terms near or adjacent to when referring to a nucleotide sequence of interest refer to a distance (e.g., number of bases) or region between the end of the primer and the nucleotide or nucleotides (e.g., nucleotide sequence) of a target. Generally, adjacent is in the range of about 1 nucleotide to about 50 nucleotides (e.g., about 1, 2, 3, 4, 5, 6, 7, 8, 9, 10, 20, 30, 40, or 50 nucleotide(s)) away from a nucleotide or nucleotide sequence of interest. In some embodiments, primers in a set (e.g., a pair of primers, a forward and a reverse primer, a first oligonucleotide and a second oligonucleotide) anneal within about 1 to 20 nucleotides from a nucleotide or nucleotide sequence of interest and produce amplified products. In some embodiments, primers anneal within a nucleotide or a nucleotide sequence of interest. After annealing, each primer is extended along the target (i.e., template strand) by a polymerase to generate a complementary strand. Several cycles of primer annealing and extension can be carried out, for example, until a detectable amount of amplification product is generated. In some embodiments, where a target nucleic acid is RNA, a DNA copy (cDNA) of the target RNA is synthesized prior to or during the amplification step by reverse transcription.

Components of an amplification reaction (e.g., the one or more amplification reagents) can include, for example, one or more primers (e.g., individual primers, primer pairs, primer sets, oligonucleotides, multiple primer sets for multiplex amplification, and the like), nucleic acid target(s) (e.g., target nucleic acid from a sample), one or more polymerases, nucleotides (e.g., dNTPs and the like), and a suitable buffer (e.g., a buffer comprising a detergent, a reducing agent, monovalent ions, and divalent ions). An amplification reaction can further include one or more of: a reverse transcriptase, a reverse transcription primer, and one or more detection agents.

Nucleic acid amplification can be conducted in the presence of native nucleotides, for example, dideoxyribonucleoside triphosphates (dNTPs), and/or derivatized nucleotides. A native nucleotide generally refers to adenylic acid, guanylic acid, cytidylic acid, thymidylic acid, or uridylic acid. A derivatized nucleotide generally is a nucleotide other than a native nucleotide. A ribonucleoside triphosphate is referred to as NTP or rNTP, where N can be A, G, C, U. A deoxynucleoside triphosphate substrates is referred to as dNTP, where N can be A, G, C, T, or U. Monomeric nucleotide subunits may be denoted as A, G, C, T, or U herein with no particular reference to DNA or RNA. In some embodiments, non-naturally occurring nucleotides or nucleotide analogs, such as analogs containing a detectable label (e.g., fluorescent or colorimetric label), may be used. For example, nucleic acid amplification can be carried out in the presence of labeled dNTPs, for example, radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, or fluorochromes. In some embodiments, nucleic acid amplification may be carried out in the presence of modified dNTPs, for example, heat activated dNTPs (e.g., CleanAmp™ dNTPs from TriLink).

The one or more amplification reagents can include non-enzymatic components and enzymatic components. Non-enzymatic components can include, for example, primers, nucleotides, buffers, salts, reducing agents, detergents, and ions. In some embodiments, the Non-enzymatic components do not include proteins (e.g., nucleic acid binding proteins), enzymes, or proteins having enzymatic activity, for example, polymerases, reverse transcriptases, helicases, topoisomerases, ligases, exonucleases, endonucleases, restriction enzymes, nicking enzymes, recombinases and the like. In some embodiments, an enzymatic component consists of a polymerase or consists of a polymerase and a reverse transcriptase. Accordingly, such enzymatic components would exclude other proteins (e.g., nucleic acid binding proteins and/or proteins having enzymatic activity), for example, helicases, topoisomerases, ligases, exonucleases, endonucleases, restriction enzymes, nicking enzymes, recombinases, and the like.

In some embodiments, amplification conditions comprise an enzymatic activity (e.g., an enzymatic activity provided by a polymerase or provided by a polymerase and a reverse transcriptase). In some embodiments, the enzymatic activity does not include enzymatic activity provided by enzymes other than the polymerase and/or the reverse transcriptase, for example, helicases, topoisomerases, ligases, exonucleases, endonucleases, restriction enzymes, nicking enzymes, recombinases, and the like. A polymerase activity and a reverse transcriptase activity can be provided by separate enzymes or separate enzyme types (e.g., polymerase(s) and reverse transcriptase(s)), or provided by a single enzyme or enzyme type (e.g., polymerase(s)).

Amplification of nucleic acid can comprise a non-thermocycling type of PCR. In some embodiments, amplification of nucleic acid comprises an isothermal amplification process, for example an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction can be kept at an essentially constant temperature, which means the temperature may not be maintained at precisely one temperature. For example, small fluctuations in temperature (e.g., ±1 to 5° C.) may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions herein can be conducted at an essentially constant temperature. In some embodiments, isothermal amplification reactions herein are conducted at a temperature of about 55° C. to a temperature of about 75° C., for example at a temperature of, or a temperature of about, 55, 56, 57, 58, 59, 60, 61, 62, 63, 64, 65, 66, 67, 68, 69, 70, 71, 72, 73, 74, or about 75° C., or a number or a range between any two of these values. In some embodiments, a temperature element (e.g., heat source) is kept at an essentially constant temperature, for example an essentially constant temperature at or below about 75° C., at or below about 70 degrees Celsius, at or below about 65° C., or at or below about 60° C.

An amplification process herein can be conducted over a certain length of time, for example until a detectable nucleic acid amplification product and/or quality control product is generated. A nucleic acid amplification product and/or quality control product may be detected by any suitable detection process and/or a detection process described herein. The amplification process can be conducted over a length of time within about 20 minutes or less, or about 10 minutes or less. For example, an amplification process can be conducted within about 1, 2, 3, 4, 5, 6, 7, 8, 9, 10, 11, 12, 13, 14, 15, 16, 17, 18, 19 or 20 minutes, or a number or a range between any two of these values.

Nucleic acid targets can be amplified without exposure to agents or conditions that denature nucleic acid, in some embodiments. Nucleic acid targets can be amplified without exposure to agents or conditions that promote strand separation during the amplification step (and/or other steps) in some embodiments. Nucleic acid targets can be amplified without exposure to agents or conditions that promote unwinding during the amplification step (and/or other steps) in some embodiments. Agents or conditions that denature nucleic acid and/or promote strand separation and/or promote unwinding may include, for example, thermal conditions (e.g., high temperatures), pH conditions (e.g., high or low pH), chemical agents, proteins (e.g., enzymatic agents), and the like.

In some embodiments, the methods disclosed herein does not comprise thermal denaturation (e.g., heating a solution containing a nucleic acid to an elevated temperature, such as, for example a temperature above 75° C., 80° C., 90° C., or 95° C., or higher) or protein-based (e.g., enzymatic) denaturation of a nucleic acid. Protein-based (e.g., enzymatic) denaturation can comprise contacting a nucleic acid with one or more of a helicase, a topoisomerase, a ligase, an exonuclease, an endonuclease, a restriction enzyme, a nicking enzyme, a recombinase, a RNA replicase, and a nucleic acid binding protein (e.g., single-stranded binding protein). In some embodiments, the compositions provided herein do not comprise a helicase, a topoisomerase, a ligase, an exonuclease, an endonuclease, a restriction enzyme, a nicking enzyme, a recombinase, a RNA replicase, and/or a nucleic acid binding protein (e.g., single-stranded binding protein). In some embodiments, the compositions and methods provided herein do not comprise intercalators, alkylating agents, and/or chemicals such as formamide, glycerol, urea, dimethyl sulfoxide (DMSO), or N,N,N-trimethylglycine (betaine). In some embodiments, the disclosed methods do not comprise contacting a nucleic acid with denaturing agents (e.g., formamide). In some embodiments, the amplifying step does not comprise agents and/or conditions that denature nucleic acids (e.g., promote strand separation and/or promote unwinding). In some embodiments, the amplifying step (e.g., step (c)) does not comprise agents and/or conditions that denature nucleic acids (e.g., promote strand separation and/or promote unwinding) other than a polymerase (e.g., a hyperthermophile polymerase). In some embodiments, the methods and compositions provided herein not comprise agents and/or conditions that denature nucleic acids (e.g., promote strand separation and/or promote unwinding) other than a polymerase (e.g., a hyperthermophile polymerase) and/or low pH conditions (e.g., contact with acid(s)).

Nucleic acid targets can be amplified without exposure to agents or conditions that promote strand separation and/or unwinding, for example a helicase, a topoisomerase, a ligase, an exonuclease, an endonuclease, a restriction enzyme, a nicking enzyme, a recombinase, a RNA replicase, a nucleic acid binding protein (e.g., single-stranded binding protein), or any combination thereof. For example, nucleic acid targets can be amplified without exposure to a helicase, including but not limited to DNA helicases and RNA helicases. Amplification conditions that do not include use of a helicase are helicase-free amplification conditions.

Nucleic acid targets can be amplified without exposure to a recombinase, including but not limited to, Cre recombinase, Hin recombinase, Tre recombinase, FLP recombinase, RecA, RAD51, RadA, T4 uvsX. In some embodiments, nucleic acid targets are amplified without exposure to a recombinase accessory protein, for example, a recombinase loading factor (e.g., T4 uvsY). Nucleic acid targets can be amplified without exposure to a nucleic acid binding protein (e.g., single-stranded binding protein or single-strand DNA-binding protein (SSB)), for example, T4 gp32. In some embodiments, nucleic acid targets are amplified without exposure to a topoisomerase. Nucleic acid targets can be amplified with or without exposure to agents or conditions that destabilize nucleic acid. As used herein, the term “destabilization” shall be given its ordinary meaning, and shall also refer to a disruption in the overall organization and geometric orientation of a nucleic acid molecule (e.g., double helical structure) by one or more of tilt, roll, twist, slip, and flip effects (e.g., as described in Lenglet et al., (2010) Journal of Nucleic Acids Volume 2010, Article ID 290935, 17 pages). Destabilization generally does not refer to melting or separation of nucleic acid strands (e.g., denaturation). Nucleic acid destabilization can be achieved, for example, by exposure to agents such as intercalators or alkylating agents, and/or chemicals such as formamide, urea, dimethyl sulfoxide (DMSO), or N,N,N-trimethylglycine (betaine). In some embodiments, methods provided herein include use of one or more destabilizing agents. In some embodiments, methods provided herein exclude use of destabilizing agents. In some embodiments, nucleic acid targets are amplified without exposure to a ligase and/or an RNA replicase.

Nucleic acid targets can be amplified without cleavage or digestion, in some embodiments. For example, nucleic acid targets can be amplified without prior exposure to one or more cleavage agents, and intact nucleic acid is amplified. In some embodiments, nucleic acid targets are amplified without exposure to one or more cleavage agents during amplification. In some embodiments, nucleic acid targets are amplified without exposure to one or more cleavage agents after amplification. Amplification conditions that do not include use of a cleavage agent may be referred to herein as cleavage agent-free amplification conditions. The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site. Cleavage agents can include endonucleases (e.g., restriction enzymes, nicking enzymes, and the like); exonucleases (DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases); and chemical cleavage agents.

Nucleic acid targets can be amplified without use of restriction enzymes and/or nicking enzymes. In some embodiments, nucleic acid is amplified without prior exposure to restriction enzymes and/or nicking enzymes. In some embodiments, nucleic acid is amplified without exposure to restriction enzymes and/or nicking enzymes during amplification. In some embodiments, nucleic acid is amplified without exposure to restriction enzymes and/or nicking enzymes after amplification. Nucleic acid targets can be amplified without exonuclease treatment. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, nucleic acid is amplified without exonuclease treatment prior to, during, and/or after amplification. Amplification conditions that do not include use of an exonuclease are exonuclease-free amplification conditions. In some embodiments, nucleic acid is amplified without DNAse treatment and/or RNAse treatment. In some embodiments, nucleic acid is amplified without RNAseH treatment.

An amplified nucleic acid may be referred to herein as a nucleic acid amplification product or amplicon. In some embodiments, the amplification product includes naturally occurring nucleotides, non-naturally occurring nucleotides, nucleotide analogs and the like and combinations of the foregoing. An amplification product typically has a nucleotide sequence that is identical to or substantially identical to a sequence in a sample nucleic acid (e.g., target sequence) or complement thereof. A “substantially identical” nucleotide sequence in an amplification product will generally have a high degree of sequence identity to the nucleotide sequence being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of polymerase infidelity or other variables.

In some embodiments, a nucleic acid amplification product comprises a polynucleotide that is continuously complementary to or substantially identical to a target sequence in sample nucleic acid. Continuously complementary generally refers to a nucleotide sequence in a first strand, for example, where each base in order (e.g., read 5′ to 3′) pairs with a correspondingly ordered base in a second strand, and there are no gaps, additional sequences or unpaired bases within the sequence considered as continuously complementary. Stated another way, continuously complementary generally refers to all contiguous bases of a nucleotide sequence in a first stand being complementary to corresponding contiguous bases of a nucleotide sequence in a second strand. A continuously complementary sequence sometimes is about 5 to about 25 contiguous bases in length, e.g., about 5, 6, 7, 8, 9, 10, 11, 12, 13, 14, 15, 16, 17, 18, 19, 20, 21, 22, 23, 24, 25, or a range between any two of these values, contiguous bases in length. In some embodiments, a nucleic acid amplification product consists of a polynucleotide that is continuously complementary to or substantially identical to a target sequence in sample nucleic acid. Accordingly, in some embodiments, a nucleic acid amplification product does not include any additional sequences (e.g., at the 5′ and/or 3′ end, or within the product) that are not continuously complementary to or substantially identical to a target sequence, for example, additional sequences incorporated into an amplification product by way of tailed primers or ligation, and/or additional sequences providing cleavage agent recognition sites (e.g., nicking enzyme recognition sites). Generally, unless a target sequence comprises tandem repeats, an amplification product does not include product in the form of tandem repeats.

Nucleic acid amplification products can comprise sequences complementary to or substantially identical to one or more primers used in an amplification reaction. In some embodiments, a nucleic acid amplification product comprises a first nucleotide sequence that is continuously complementary to or identical to a first primer sequence, and a second nucleotide sequence that is continuously complementary to or identical to a second primer sequence.

Nucleic acid amplification products can comprise a spacer sequence. As described herein, a spacer sequence in an amplification product is a sequence (1 or more bases) continuously complementary to or substantially identical to a portion of a target sequence in the sample nucleic acid, and is flanked by sequences in the amplification product that are complementary to or substantially identical to one or more primers used in an amplification reaction. A spacer sequence flanked by sequences in the amplification product generally lies between a first sequence (complementary to or substantially identical to a first primer) and a second sequence (complementary to or substantially identical to a second primer). Thus, an amplification product typically includes a first sequence followed by a spacer sequences followed by a second sequence. A spacer sequence generally is not complementary to or substantially identical to a sequence in the primer(s). A spacer sequence can be, or can comprise, about 1 to 10 bases, including 1, 2, 3, 4, 5, 6, 7, 8, 9, or 10 bases. In some embodiments, a nucleic acid amplification product consists of, or consists essentially of, a first nucleotide sequence that is continuously complementary to or identical to a first primer sequence, a second nucleotide sequence that is continuously complementary to or identical to a second primer sequence, and a spacer sequence. In some embodiments, a nucleic acid amplification product does not include any additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. In some embodiments, a nucleic acid amplification product generally does not include additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. However, in such embodiments, a nucleic acid amplification product may include, for example, some mismatched (i.e., non-complementary) bases or one more extra bases (e.g., at the 5′ and/or 3′ end; or within the product) introduced into the product by way of error or promiscuity in the amplification process.

Nucleic acid amplification products can be up to 50 bases in length, including 10, 15, 20, 25, 30, 35, 40, 45, 50, or a number or a range between any two of these values, bases long. In some embodiments, nucleic acid amplification products for a given target sequence have the same length or substantially the same length (e.g., within 1 to 10 bases). Accordingly, nucleic acid amplification products for a given target sequence may produce a single signal (e.g., band on an electrophoresis gel) and generally do not produce multiple signals indicative of multiple lengths (e.g., a ladder or smear on an electrophoresis gel). For multiplex reactions, nucleic acid amplification products for different target sequences may have different lengths.

The methods and components described herein can be used for multiplex amplification which generally refers to the amplification of more than one nucleic acid of interest (e.g., amplification or more than one target sequence). For example, multiplex amplification can refer to amplification of multiple sequences from the same sample or amplification of one of several sequences in a sample. For example, the amplifying step can comprise multiplex amplification of two or more target nucleic acid sequences and the detecting step can comprise multiplex detection of two or more nucleic acid amplification products derived from said two or more target nucleic acid sequences. The two or more target nucleic acid sequences can be specific to two or more different organisms (e.g., one or more of SARS-CoV-2, Influenza A, Influenza B, and/or Influenza C). Multiplex amplification also can refer to amplification of one or more sequences present in multiple samples either simultaneously or in step-wise fashion. For example, a multiplex amplification can be used for amplifying least two target sequences that are capable of being amplified (e.g., the amplification reaction comprises the appropriate primers and enzymes to amplify at least two target sequences). In some embodiments, an amplification reaction is prepared to detect at least two target sequences, but only one of the target sequences is present in the sample being tested, such that both sequences are capable of being amplified, but only one sequence is amplified. In some embodiments, where two target sequences are present, an amplification reaction results in the amplification of both target sequences. A multiplex amplification reaction can result in the amplification of one, some, or all of the target sequences for which it comprises the appropriate primers and enzymes. In some embodiments, an amplification reaction is prepared to detect two sequences with one pair of primers, where one sequence is a target sequence and one sequence is a control sequence (e.g., a synthetic sequence capable of being amplified by the same primers as the target sequence and having a different spacer base or sequence than the target). In some embodiments, an amplification reaction is prepared to detect multiple sets of sequences with corresponding primer pairs, where each set includes a target sequence and a control sequence.

### Primers

Nucleic acid amplification generally is conducted in the presence of one or more primers. A primer is generally characterized as an oligonucleotide that includes a nucleotide sequence capable of hybridizing or annealing to a target nucleic acid, at or near (e.g., adjacent to) a specific region of interest (i.e., target sequence). Primers can allow for specific determination of a target nucleic acid nucleotide sequence or detection of the target nucleic acid (e.g., presence or absence of a sequence), or feature thereof, for example. A primer can be naturally occurring or synthetic. The term specific, or specificity, generally refers to the binding or hybridization of one molecule to another molecule, such as a primer for a target polynucleotide. That is, specific or specificity refers to the recognition, contact, and formation of a stable complex between two molecules, as compared to substantially less recognition, contact, or complex formation of either of those two molecules with other molecules. The term anneal or hybridize generally refers to the formation of a stable complex between two molecules. The terms primer, oligo, or oligonucleotide may be used interchangeably herein, when referring to primers.

A primer can be designed and synthesized using suitable processes, and can be of any length suitable for hybridizing to a target sequence and performing an amplification process described herein. Primers often are designed according to a sequence in a target nucleic acid. A primer in some embodiments may be about 5 to about 30 bases in length, for example 5, 6, 7, 8, 9, 10, 11, 12, 13, 14, 15, 16, 17, 18, 19, 20, 21, 22, 23, 24, 25, 26, 27, 28, 29 or 30 bases in length. A primer may be composed of naturally occurring and/or non-naturally occurring nucleotides (e.g., modified nucleotides, labeled nucleotides), or a mixture thereof. Modifications and modified bases may include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S—S, dithiol, thiol modifier C6 S—S)); fluorophores (e.g., Freedom™ Dyes, Alexa Fluor® Dyes, LI-COR IRDyes®, ATTO™ Dyes, Rhodamine Dyes, WellRED Dyes, 6-FAM (azide), Texas Red®-X (NHS ester), Lightcycler® 640 (NHS ester), Dy 750 (NHS ester)); Iowa Black® dark quenchers modifications (e.g., Iowa Black® FQ, Iowa Black® RQ); dark quenchers modifications (e.g., Black Hole Quencher®-1, Black Hole Quencher®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxyInosine, Super T®, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate (PS) bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, PS linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tm of RNA:RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate (PS) linkages (e.g., PS bond modifications). A PS bond substitutes a sulfur atom for a non-bridging oxygen in the phosphate backbone of a primer. This modification typically renders the internucleotide linkage resistant to nuclease degradation. PS bonds can be introduced between about the last 3 to 5 nucleotides at the 5′-end or the 3′-end of a primer to inhibit exonuclease degradation, for example. PS bonds included throughout an entire primer can help reduce attack by endonucleases, in some embodiments. A primer can, for example, comprise a 3′ phosphate group. 3′ phosphorylation can inhibit degradation by certain 3′-exonucleases and can be used to block extension by DNA polymerases, in certain instances. In some embodiments, a primer comprises one or more spacer bases (e.g., one or more C3 spacers). A C3 spacer phosphoramidite can be incorporated internally or at the 5′-end of a primer. Multiple C3 spacers can be added at either end of a primer to introduce a long hydrophilic spacer arm for the attachment of fluorophores or other pendent groups, for example.

A primer can comprises DNA bases, RNA bases, or both, where one or more of the DNA bases and RNA bases is modified or unmodified. For example, a primer can be a mixture of DNA bases and RNA bases. The primer can consist of DNA bases (e.g., modified DNA bases and/or unmodified DNA bases). In some embodiments, the primer consists of unmodified DNA bases. In some embodiments, the primer consists of modified DNA bases. The primer can consist of RNA bases (e.g., modified RNA bases and/or unmodified RNA bases). In some embodiments, the primer consists of unmodified RNA bases. In some embodiments, the primer consists of modified RNA bases. In some embodiments, a primer comprises no RNA bases. In some embodiments, a primer comprises no DNA bases. In some embodiments, the primer comprises no cleavage agent recognition sites (e.g., no nicking enzyme recognition sites). In some embodiments, a primer comprises no tail (e.g., no tail comprising a nicking enzyme recognition site).

All or a portion of a primer sequence can be complementary or substantially complementary to a target nucleic acid, in some embodiments. Substantially complementary with respect to sequences generally refers to nucleotide sequences that will hybridize with each other. The stringency of the hybridization conditions can be altered to tolerate varying amounts of sequence mismatch. The target and primer sequences can be, for example, at least 75% complementary to each other, including 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to each other. Primers that are substantially complimentary to a target nucleic acid sequence typically are also substantially identical to the complement of the target nucleic acid sequence (i.e., the sequence of the anti-sense strand of the target nucleic acid). The primer and the anti-sense strand of the target nucleic acid can be at least 75% identical in sequence, for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each other.

In some embodiments, primers comprise a pair of primers. A pair of primers may include a forward primer and a reverse primer (e.g., primers that bind to the sense and antisense strands of a target nucleic acid). In some embodiments, primers consist of a pair of primers (i.e. a forward primer and a reverse primer). Accordingly, in some embodiments, amplification of a target sequence is performed using a pair of primers and no additional primers or oligonucleotides are included in the amplification of the target sequence (e.g., the amplification reaction components comprise no additional primer pairs for a given target sequence, no nested primers, no bumper primers, no oligonucleotides other than the primers, no probes, and the like). In some embodiments, primers consist of a pair of primers. In some embodiments, an amplification reaction can include additional primer pairs for amplifying different target sequences, such as in a multiplex amplification. In some embodiments, primers consist of a pair of primers, however, in some embodiments, an amplification reaction can include additional primers, oligonucleotides or probes for a detection process that are not considered part of amplification. In some embodiments, primers are used in sets. An amplification primer set can include a pair of forward and reverse primers for a given target sequence. For multiplex amplification, primers that amplify a first target sequence are considered a primer set, and primers that amplify a second target sequence are considered a different primer set.

Nucleic acids described herein (e.g., amplification products, sample nucleic acids, target nucleic acid sequences) can comprise a first strand and a second strand complementary to each other. Amplification reaction components can comprise, or consist of, a first primer (first oligonucleotide) complementary to a target sequence in a first strand (e.g., sense strand, forward strand) of a sample nucleic acid, and a second primer (second oligonucleotide) complementary to a target sequence in a second strand (e.g., antisense strand, reverse strand) of a sample nucleic acid. In some embodiments, a first primer (first oligonucleotide) comprises a first polynucleotide continuously complementary to a target sequence in a first strand of sample nucleic acid, and a second primer (second oligonucleotide) comprises a second polynucleotide continuously complementary to a target sequence in a second strand of sample nucleic acid. Continuously complementary for a primer-target generally refers to a nucleotide sequence in a primer, where each base in order pairs with a correspondingly ordered base in a target sequence, and there are no gaps, additional sequences or unpaired bases within the sequence considered as continuously complementary. In some embodiments, a primer does not include any additional sequences (e.g., at the 5′ and/or 3′ end, or within the primer) that are not continuously complementary to a target sequence, for example, additional sequences present in tailed primers or looped primers, and/or additional sequences providing cleavage agent recognition sites (e.g., nicking enzyme recognition sites). In some embodiments, amplification reaction components do not comprise primers comprising additional sequences (i.e., sequences other than the sequence that is continuously complementary to a target sequence), for example, tailed primers, looped primers, primers capable of forming step-loop structures, hairpin structures, and/or additional sequences providing cleavage agent recognition sites (e.g., nicking enzyme recognition sites), and the like.

The primer, in some embodiments, can contain a modification such as one or more inosines, abasic sites, LNAs, minor groove binders, duplex stabilizers (e.g., acridine, spermidine), Tm modifiers or any modifier that changes the binding properties of the primer. The primer, in some embodiments, can contain a detectable molecule or entity (e.g., a fluorophore, radioisotope, colorimetric agent, particle, enzyme and the like).

### Polymerase

Amplification reaction components (e.g., one or more amplification reagents) can comprise one or more polymerases. Polymerases are proteins capable of catalyzing the specific incorporation of nucleotides to extend a 3′ hydroxyl terminus of a primer molecule, for example, an amplification primer described herein, against a nucleic acid target sequence (e.g., to which a primer is annealed). Non-limiting examples of polymerases include thermophilic or hyperthermophilic polymerases that can have activity at an elevated reaction temperature (e.g., above 55, 60, 65, 70, 75, 80, 85, 90, 95, or 100° C.). A hyperthermophilic polymerase may be referred to as a hyperthermophile polymerase. A polymerase may or may not have strand displacement capabilities. In some embodiments, a polymerase can incorporate about 1 to about 50 nucleotides in a single synthesis, for example about 1, 5, 10, 15, 20, 25, 30, 35, 40, 45 or 50 nucleotides, or a number or a range between any two of these values, in a single synthesis.

The amplification reaction components can comprise one or more DNA polymerases selected from: 9° N DNA polymerase; 9° Nm™ DNA polymerase; Therminator™ DNA Polymerase; Therminator™ II DNA Polymerase; Therminator™ III DNA Polymerase; Therminator™ γ DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; Deep VentR™ (exo-) DNA Polymerase; Deep VentR™ DNA Polymerase; DyNAzyme™ EXT DNA; DyNAzyme™ II Hot Start DNA Polymerase; Phusion™ High-Fidelity DNA Polymerase; VentR® DNA Polymerase; VentR® (exo-) DNA Polymerase; RepliPHI™ Phi29 DNA Polymerase; rBst DNA Polymerase, large fragment (IsoTherm™ DNA Polymerase); MasterAmp™ AmpliTherm™ DNA Polymerase; Taq DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, the amplification reaction components comprise one or more hyperthermophile DNA polymerases (e.g., hyperthermophile DNA polymerases that are thermostable at high temperatures). The hyperthermophile DNA polymerase can have a half-life of about 5 to 10 hours at 95° C. and a half-life of about 1 to 3 hours at 100° C. For example, the amplification reaction components can comprise one or more hyperthermophile DNA polymerases from Archaea (e.g., hyperthermophile DNA polymerases from Thermococcus, or hyperthermophile DNA polymerases from Thermococcaceaen archaean). In some embodiments, amplification reaction components comprise one or more hyperthermophile DNA polymerases from Pyrococcus, Methanococcaceae, Methanococcus, or Thermus. In some embodiments, amplification reaction components comprise one or more hyperthermophile DNA polymerases from Thermus thermophiles.

In some embodiments, amplification reaction components comprise a hyperthermophile DNA polymerase or functional fragment thereof. A functional fragment generally retains one or more functions of a full-length polymerase, for example, the capability to polymerize DNA (e.g., in an amplification reaction). In some instances, a functional fragment performs a function (e.g., polymerization of DNA in an amplification reaction) at a level that is at least about 50%, at least about 75%, at least about 90%, at least about 95% the level of function for a full length polymerase. Levels of polymerase activity can be assessed, for example, using a detectable nucleic acid amplification method, such as a method described herein. In some embodiments, amplification reaction components comprise a hyperthermophile DNA polymerase comprising an amino acid sequence of SEQ ID NO: 1 or SEQ ID NO: 2, or a functional fragment of SEQ ID NO: 1 or SEQ ID NO: 2.

In some embodiments, amplification reaction components (e.g., one or more amplification reagents) comprise a polymerase comprising an amino acid sequence that is at least about 90% identical to a hyperthermophile polymerase or a functional fragment thereof. In some embodiments, amplification reaction components comprise a polymerase comprising an amino acid sequence that is at least about 90%, 95%, or 99% identical to the amino acid sequence of SEQ ID NO: 1 or SEQ ID NO: 2, or a functional fragment thereof.

The polymerase can possess reverse transcription capabilities. In such embodiments, the amplification reaction can amplify RNA targets, for example, in a single step without the use of a separate reverse transcriptase. Non-limiting examples of polymerases that possess reverse transcriptase capabilities include Bst (large fragment), 9° N DNA polymerase, 9° Nm™ DNA polymerase, Therminator™, Therminator™ II, and the like). Amplification reaction components can comprise one or more separate reverse transcriptases. In some embodiments, more than one polymerase is included in in an amplification reaction. For example, an amplification reaction may comprise a polymerase having reverse transcriptase activity and a second polymerase having no reverse transcriptase activity.

In some embodiments, one or more polymerases having exonuclease activity are used during amplification. In some embodiments, one or more polymerases having no or low exonuclease activity are used during amplification. In some embodiments, a polymerase having no or low exonuclease activity comprises one or more modifications (e.g., amino acid substitutions) that reduce or eliminate the exonuclease activity of the polymerase. For example, a modified polymerase having low exonuclease activity can have 10% or less exonuclease activity compared to an unmodified polymerase, for example less than about 10%, 9%, 8%, 7%, 6%, 5%, 4%, 3%, 2%, or 1% exonuclease activity compared to an unmodified polymerase. In some embodiments, a polymerase has no or low 5′ to 3′ exonuclease activity, and/or no or low 3′ to 5′ exonuclease activity. In some embodiments, a polymerase has no or low single strand dependent exonuclease activity, and/or no or low double strand dependent exonuclease activity. Nonlimiting examples of the modifications that can reduce or eliminate exonuclease activity for a polymerase include one or more amino acid substitutions at position 141 and/or 143 and/or 458 of SEQ ID NO: 1 (e.g., D141A, E143A, E143D and A485L), or at a position corresponding to position 141 and/or 143 and/or 458 of SEQ ID NO: 1.

### Detection and Quantification

The methods described herein can comprise detecting and/or quantifying nucleic acid amplification product(s) and/or quality control product(s). Amplification product(s) can be detected and/or quantified, for example, by any suitable detection and/or quantification method described herein (e.g., signal-generating oligonucleotides). Non-limiting examples of detection and/or quantification methods include molecular beacon (e.g., real-time, endpoint), lateral flow, fluorescence resonance energy transfer (FRET), fluorescence polarization (FP), surface capture, 5′ to 3′ exonuclease hydrolysis probes (e.g., TAQMAN), intercalating/binding dyes, absorbance methods (e.g., colorimetric, turbidity), electrophoresis (e.g., gel electrophoresis, capillary electrophoresis), mass spectrometry, nucleic acid sequencing, digital amplification, a primer extension method (e.g., iPLEX™), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and LNA probes, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), nanopore sequencing, chips and combinations thereof. In some embodiments, detecting a nucleic acid amplification product and/or quality control product comprises use of a real-time detection method (i.e., product is detected and/or continuously monitored during an amplification process). In some embodiments, detecting a nucleic acid amplification product comprises use of an endpoint detection method (i.e., product is detected after completing or stopping an amplification process). Nucleic acid detection methods may also employ the use of labeled nucleotides incorporated directly into a target sequence or into probes containing complementary sequences to a target. Such labels may be radioactive and/or fluorescent in nature and can be resolved in any of the manners discussed herein. In some embodiments, quantification of a nucleic acid amplification product may be achieved using one or more detection methods described below. In some embodiments, the detection method can be used in conjunction with a measurement of signal intensity, and/or generation of (or reference to) a standard curve and/or look-up table for quantification of a nucleic acid amplification product and/or quality control product.

Detecting a nucleic acid amplification product and/or quality control product can comprise use of molecular beacon technology. The term molecular beacon generally refers to a detectable molecule, where the detectable property of the molecule is detectable under certain conditions, thereby enabling the molecule to function as a specific and informative signal. Non-limiting examples of detectable properties include optical properties (e.g., fluorescence), electrical properties, magnetic properties, chemical properties and time or speed through an opening of known size. Molecular beacons for detecting nucleic acid molecules can be, for example, hair-pin shaped oligonucleotides containing a fluorophore on one end and a quenching dye on the opposite end. The loop of the hair-pin can contain a probe sequence that is complementary to a target sequence and the stem is formed by annealing of complementary arm sequences located on either side of the probe sequence. A fluorophore and a quenching molecule can be covalently linked at opposite ends of each arm. Under conditions that prevent the oligonucleotides from hybridizing to its complementary target or when the molecular beacon is free in solution, the fluorescent and quenching molecules are proximal to one another preventing FRET. When the molecular beacon encounters a target molecule (e.g., a nucleic acid amplification product and/or quality control product), hybridization can occur, and the loop structure is converted to a stable more rigid conformation causing separation of the fluorophore and quencher molecules leading to fluorescence. Due to the specificity of the probe, the generation of fluorescence generally is exclusively due to the synthesis of the intended amplified product. In some instances, a molecular beacon probe sequence hybridizes to a sequence in an amplification product that is identical to or complementary to a sequence in a target nucleic acid. In some instances, a molecular beacon probe sequence hybridizes to a sequence in an amplification product that is not identical to or complementary to a sequence in a target nucleic acid (e.g., hybridizes to a sequence added to an amplification product by way of a tailed amplification primer or ligation). Molecular beacons are highly specific and can discern a single nucleotide polymorphism. Molecular beacons also can be synthesized with different colored fluorophores and different target sequences, enabling simultaneous detection of several products in the same reaction (e.g., in a multiplex reaction). For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. Detection using molecular beacons can be done in real time or as an end-point detection method.

Detecting a nucleic acid amplification product and/or quality control product can comprise use of lateral flow. Use of lateral flow typically includes use of a lateral flow device including but not limited to dipstick assays and thin layer chromatographic plates with various appropriate coatings. Immobilized on the flow path are various binding reagents for the sample, binding partners or conjugates involving binding partners for the sample and signal producing systems.

Detecting a nucleic acid amplification product and/or quality control product can comprise use of FRET which is an energy transfer mechanism between two chromophores: a donor and an acceptor molecule. Briefly, a donor fluorophore molecule is excited at a specific excitation wavelength. The subsequent emission from the donor molecule as it returns to its ground state may transfer excitation energy to the acceptor molecule through a long range dipole-dipole interaction. The emission intensity of the acceptor molecule can be monitored and is a function of the distance between the donor and the acceptor, the overlap of the donor emission spectrum and the acceptor absorption spectrum and the orientation of the donor emission dipole moment and the acceptor absorption dipole moment. FRET can be useful for quantifying molecular dynamics, for example, in DNA-DNA interactions as described for molecular beacons. For monitoring the production of a specific product, a probe can be labeled with a donor molecule on one end and an acceptor molecule on the other. Probe-target hybridization brings a change in the distance or orientation of the donor and acceptor and FRET change is observed.

Detecting a nucleic acid amplification product and/or quality control product can comprise use of fluorescence polarization (FP). FP techniques are based on the principle that a fluorescently labeled compound when excited by linearly polarized light will emit fluorescence having a degree of polarization inversely related to its rate of rotation. Therefore, when a molecule such as a tracer-nucleic acid conjugate, for example, having a fluorescent label is excited with linearly polarized light, the emitted light remains highly polarized because the fluorophore is constrained from rotating between the time light is absorbed and emitted. When a free tracer compound (i.e., unbound to a nucleic acid) is excited by linearly polarized light, its rotation is much faster than the corresponding tracer-nucleic acid conjugate and the molecules are more randomly oriented, therefore, the emitted light is depolarized. Thus, fluorescence polarization provides a quantitative means for measuring the amount of tracer-nucleic acid conjugate produced in an amplification reaction.

Detecting a nucleic acid amplification product and/or quality control product can comprise use of surface capture, accomplished for example by the immobilization of specific oligonucleotides to a surface producing a biosensor that is both highly sensitive and selective. Example surfaces that can be used for attaching the probe include gold and carbon. Detecting a nucleic acid amplification product and/or quality control product can comprise use of 5′ to 3′ exonuclease hydrolysis probes (e.g., TAQMAN). TAQMAN probes, for example, are hydrolysis probes that can increase the specificity of a quantitative amplification method (e.g., quantitative PCR). The TAQMAN probe principle relies on 1) the 5′ to 3′ exonuclease activity of Taq polymerase to cleave a dual-labeled probe during hybridization to a complementary target sequence and 2) fluorophore-based detection. A resulting fluorescence signal permits quantitative measurements of the accumulation of amplification product during the exponential stages of amplification, and the TAQMAN probe can significantly increase the specificity of the detection.

Detecting a nucleic acid amplification product and/or quality control product can comprise use of intercalating and/or binding dyes, including dyes that specifically stain nucleic acid (e.g., intercalating dyes exhibit enhanced fluorescence upon binding to DNA or RNA). Dyes can include DNA or RNA intercalating fluorophores, including but not limited to, SYTO® 82, acridine orange, ethidium bromide, Hoechst dyes, PicoGreen®, propidium iodide, SYBR® I (an asymmetrical cyanine dye), SYBR® II, TOTO (a thiaxole orange dimer) and YOYO (an oxazole yellow dimer). Detecting a nucleic acid amplification product and/or quality control product can comprise use of absorbance methods (e.g., colorimetric, turbidity). In some embodiments, detection and/or quantitation of nucleic acid can be achieved by directly converting absorbance (e.g., UV absorbance measurements at 260 nm) to concentration. Direct measurement of nucleic acid can be converted to concentration using the Beer Lambert law which relates absorbance to concentration using the path length of the measurement and an extinction coefficient. Detecting a nucleic acid amplification product and/or quality control product can comprise use of electrophoresis (e.g., gel electrophoresis, capillary electrophoresis) and/or use of mass spectrometry. Mass Spectrometry is an analytical technique that can be used to determine the structure and quantity of a nucleic acid and can be used to provide rapid analysis of complex mixtures. Following amplification, samples can be ionized, the resulting ions separated in electric and/or magnetic fields according to their mass-to-charge ratio, and a detector measures the mass-to-charge ratio of ions. Mass spectrometry methods include, for example, MALDI, MALDI-TOF, and electrospray. These methods may be combined with gas chromatography (GC/MS) and liquid chromatography (LC/MS). Mass spectrometry (e.g., matrix-assisted laser desorption/ionization mass spectrometry (MALDI MS)) can be high throughput due to high-speed signal acquisition and automated analysis off solid surfaces.

Detecting a nucleic acid amplification product and/or quality control product can comprise use of nucleic acid sequencing. The entire sequence or a partial sequence of an amplification product can be determined, and the determined nucleotide sequence may be referred to as a read. For example, linear amplification products may be analyzed directly without further amplification (e.g., by using single-molecule sequencing methodology). In some embodiments, linear amplification products are subject to further amplification and then analyzed (e.g., using sequencing by ligation or pyrosequencing methodology). Non-limiting examples of sequencing methods include single-end sequencing, paired-end sequencing, reversible terminator-based sequencing, sequencing by ligation, pyrosequencing, sequencing by synthesis, single-molecule sequencing, multiplex sequencing, solid phase single nucleotide sequencing, and nanopore sequencing. Detecting a nucleic acid amplification product and/or quality control product can comprise use of digital amplification (e.g., digital PCR). Systems for digital amplification and analysis of nucleic acids are available (e.g., Fluidigm® Corporation).

### Lysis Buffers

### Lytic Agents

As disclosed herein, the lytic agents can comprise a detergent. The detergent can comprise one or more of a cationic surfactant, an anionic surfactant, a non-ionic surfactant, and an amphoteric surfactant. The anionic surfactant can comprise NH₄⁺, K⁺, Na⁺, or Li⁺ as a counter ion. The cationic surfactant can comprise I⁻, Br⁻, or Cl⁻ as a counter ion.

The lytic agents provided herein can be capable of acting as a denaturing agent. “Denaturing agent” or “denaturant,” as used herein, shall be given its ordinary meaning and include any compound or material which will cause a reversible unfolding of a protein. The strength of a denaturing agent or denaturant will be determined both by the properties and the concentration of the particular denaturing agent or denaturant. Suitable denaturing agents or denaturants include chaotropes, detergents, organic solvents, water miscible solvents, phospholipids, or a combination of two or more such agents. Suitable chaotropes include, but are not limited to, urea, guanidine, and sodium thiocyanate. Useful detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents (e.g., N->2,3-(Dioleyoxy)-propyl-N,N,N-trimethylammonium), mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Organic, water miscible solvents such as acetonitrile, lower alkanols (especially C₂-C₄ alkanols such as ethanol or isopropanol), or lower alkandiols (especially C₂-C₄ alkandiols such as ethylene-glycol) may be used as denaturants. Phospholipids can be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

Suitable surfactant levels can be from about 0.1% to about 25%, from about 0.25% to about 10%, or from about 0.5% to about 5% by weight of the total composition. In some embodiments, the surfactants are anionic surfactants, amphoteric surfactants, nonionic surfactants, zwitterionic surfactants, cationic surfactants, and mixtures thereof. In some embodiments, it can be advantageous to use anionic, amphoteric, nonionic and zwitterionic surfactants (and mixtures thereof).

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; cetyl trimethylammonium bromide; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions, methods and kits of the present disclosure can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilizing foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilizing group, e.g., carboxy, sulphonate, sulphate, phosphate or phosphonate.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

### Reducing Agents

The lysis buffer and/or reagent composition (e.g., dried composition) can comprise one or more reducing agents. A “reducing agent” can be a compound or a group of compounds. As used herein, “reducing agent”, also known as “reductant,” “reducer,” or “reducing equivalent,” can refer to an element or compound that donates an electron to another species. In particular, a reducing agent is generally a compound that breaks disulfide bonds by reduction, thereby overcoming those tertiary protein folding and quaternary protein structures (oligomeric subunits) which are stabilized by disulfide bonds. Examples of a suitable reducing agent include, but are not limited to, 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED. In some embodiments of the methods, compositions and kits provided herein, the lysis buffer and/or reagent composition (e.g., dried composition) does not comprise one or more reducing agents.

### Reagent Composition

The reagent compositions described herein (e.g., dried composition) can be provided in a “dry form,” or in a form not suspended in liquid medium. The “dry form” of the compositions can include dry powders, lyophilized compositions, spray-dried, or precipitated compositions. The “dry form” compositions can include one or more lyoprotectants, such as sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. As used herein, the terms “lyophilization,” “lyophilized,” and “freeze-dried” refer to a process by which the material to be dried is first frozen and then the ice or frozen solvent is removed by sublimation in a vacuum environment. “Lyophilisate” refers to a lyphophilized substance.

The reagent composition (e.g., dried composition) can be frozen or lyophilized or spray dried. The reagent composition can be heat dried. The reagent composition can comprise one or more additives (e.g., an amino acid, a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. Lyophilized reagents can include poly rA, EGTA, EDTA, Tween 80, and/or Tween 20.

The frozen or lyophilized or spray dried or heat dried composition or the aqueous composition for preparing the frozen or lyophilized or spray dried composition may comprise one or more of the following: (i) Non-aqueous solvents such as ethylene glycol, glycerol, dimethylsulphoxide, and dimethylformamide. (ii) Surfactants such as Tween 80, Brij 35, Brij 30, Lubrol-px, Triton X-10; Pluronic F127 (polyoxyethylene-polyoxypropylene copolymer) also known as poloxamer, poloxamine, and sodium dodecyl sulfate. (iii) Dissacharides such as trehalose, sucrose, lactose, and maltose. (iv) Polymers (which may have different MWs) such as polyethylene glycol, dextran, polyvinyl alcohol), hydroxypropyl methylcellulose, gelatin, polyvinylpyrrolidone, hydroxyethyl cellulose, Ficoll, and albumin. (v) Amino acids such as glycine, proline, 4-hydroxyproline, L-serine, glutamate, alanine, lysine, sarcosine, and gamma-aminobutyric acid.

The reagent composition (e.g., dried composition) can comprise one or more protectants and one or more amplification reagents. The one or more protectants can comprise a cyclodextrin compound. Cyclodextrins (CD) can be employed for complexation with lytic agents (e.g., SDS). Cyclodextrins (CDs) can be cyclic oligosaccharides which resemble truncated cones with hydrophobic inner cavity and hydrophilic outer surface The most commonly used natural cyclodextrins include 6, 7, and 8 glucose units, named as α, β and γ-CD. Natural CDs have can have solubility. Chemical modified CDs such as hydroxypropyl derivatives improve solubility up to 50% in aqueous media. CAVASOL® is the trade name of WACKER's cyclodextrin derivatives, which covers a variety of α, β and γ-CD derivatives. β-CD can form a strong inclusion complex (more so than α-CD and β-CD) with sodium dodecyl sulfate (SDS) in a predominately 1:1 stoichiometry. The binding constant of β-CD to SDS can range from 2100 M⁻¹ to 2500 M⁻¹.

### Kits

Disclosed herein include kits for monitoring an amplification reaction. In some embodiments, the kit comprises: a quality control template disclosed herein; a quality control primer disclosed herein; a signal-generating oligonucleotide disclosed herein; and/or a supplemental quality control primer disclosed herein. The kit can comprise: a reagent composition comprising one or more amplification reagents comprising one or more components for amplifying the target nucleic acid sequence under isothermal amplification conditions (e.g., a forward primer, a reverse primer). In some embodiments, the quality control template, the signal-generating oligonucleotide, the quality control primer, the supplemental quality control primer, and/or the one or more components for amplifying, are in a lyophilized or freeze-dried form and/or are present in the reagent composition.

The kit can comprise: at least one component providing real-time detection activity for a nucleic acid amplification product and/or quality control product. The real-time detection activity can be provided by a molecular beacon. The reagent composition (e.g., dried composition) can comprise a reverse transcriptase and/or a reverse transcription primer.

In some embodiments, the molar ratio of the one or more protectants to the one or more amplification reagents is between about 10:1 to about 1:10 (e.g., about 2:1). In some embodiments, the one or more additives comprise Tween 20, Triton X-100, Tween 80, a non-ionic detergent (e.g., a non-ionic surfactant), or any combination thereof. In some embodiments, the one or more protectants comprises a cyclodextrin compound. In some embodiments, the one or more lytic reagents comprise about 0.001% (w/v) to about 1.0% (w/v) (e.g., about 0.2% (w/v)) of the treated sample. In some embodiments, the one or more lytic agents comprise a detergent. The detergent can comprise one or more of a cationic surfactant, an anionic surfactant, a non-ionic surfactant, and an amphoteric surfactant. In some embodiments, it can be advantageous that the one or more protectants are capable of sequestering the one or more lytic agents, thereby preventing the denaturing of the one or more amplification reagents by the one or more lytic agents.

Kits can comprise, for example, one or more polymerases and one or more primers, and optionally one or more reverse transcriptases and/or reverse transcription primers, as described herein. Where one target is amplified, a pair of primers (forward and reverse) can be included in the kit. Where multiple target sequences are amplified, a plurality of primer pairs can be included in the kit. A kit can include a control polynucleotide, and where multiple target sequences are amplified, a plurality of control polynucleotides can be included in the kit.

The enzyme having a hyperthermophile polymerase activity can have an amino acid sequence that is at least about 90% or 95% identical to the amino acid sequence of SEQ ID NO: 1 or a functional fragment thereof. For example, the enzyme having a hyperthermophile polymerase activity can comprise the amino acid sequence of SEQ ID NO: 1.

The nucleic acid amplification product can be about 20 to 40 bases long. The nucleic acid amplification product can comprise: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The biological entities can comprise one or more of prokaryotic cells, eukaryotic cells, viral particles, exosomes, protoplasts, and microvesicles. The biological entities can comprise a virus, a bacteria, a fungi, a protozoa, portions thereof, or any combination thereof. The target nucleic acid sequence can be a nucleic acid sequence of a virus, bacteria, fungi, or protozoa. The sample nucleic acids can be derived from a virus, bacteria, fungi, or protozoa.

Kits can also comprise one or more of the components in any number of separate vessels, chambers, containers, packets, tubes, vials, microtiter plates and the like, or the components can be combined in various combinations in such containers. Components of the kit can, for example, be present in one or more containers. In some embodiments, all of the components are provided in one container. In some embodiments, the enzymes (e.g., polymerase(s) and/or reverse transcriptase(s)) can be provided in a separate container from the primers. The components can, for example, be lyophilized, heat dried, freeze dried, or in a stable buffer. In some embodiments, polymerase(s) and/or reverse transcriptase(s) are in lyophilized form or heat dried form in a single container, and the primers are either lyophilized, heat dried, freeze dried, or in buffer, in a different container. In some embodiments, polymerase(s) and/or reverse transcriptase(s), and the primers are, in lyophilized form or heat dried form, in a single container.

Kits can comprise, for example, dNTPs used in the reaction, or modified nucleotides, vessels, cuvettes or other containers used for the reaction, or a vial of water or buffer for re-hydrating lyophilized or heat-dried components. The buffer used can, for example, be appropriate for both polymerase and primer annealing activity. Kits can also comprise instructions for performing one or more methods described herein and/or a description of one or more components described herein. Instructions and/or descriptions can be in printed form and can be included in a kit insert. A kit also can include a written description of an internet location that provides such instructions or descriptions. Kits can comprise reagents used for detection methods, for example, reagents used for FRET, lateral flow devices, dipsticks, fluorescent dye, colloidal gold particles, latex particles, a molecular beacon, or polystyrene beads.

### EXAMPLES

Some aspects of the embodiments discussed above are disclosed in further detail in the following examples, which are not in any way intended to limit the scope of the present disclosure.

### Example 1

### Hairpin Internal Control (HPIC) Proof of Principle

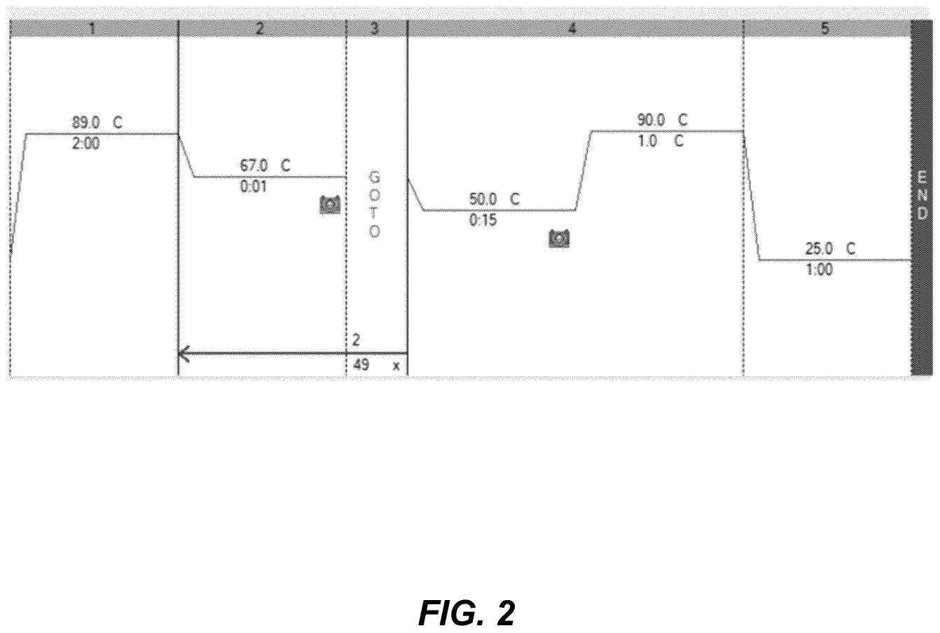

This example demonstrates the monitoring of an amplification reaction using the methods and compositions described herein. FIG. 1 depicts a non-limiting exemplary embodiment of a DNA hairpin internal control assay reaction disclosed herein and tested in this Example—a two-component MB-IC hairpin approach. An IC primer extends on a molecular beacon (e.g., hairpin-shaped probe), ultimately yielding the generation of two hairpin products which have same stem sequence and are complementary only in the loop (spacer) region. Real-time detection of hairpin products can be achieved by the same molecular beacon. The HPIC assay components are one IC primer and a molecular beacon IC (Table 1). FIG. 2 depicts thermal settings for the Hairpin IC (HPIC) isothermal amplification reactions described in this Example. In the reactions described in this Example, the molecular beacon IC (HPIC) was detected via HEX fluorophore and the target (Neisseria gonorrhoeae (Ng)) was detected via ROX fluorophore. The IC template employed is also shown in Table 1. In some embodiments, the probes (e.g., molecular beacons) provided herein comprise a 5′ modification (e.g., 5HEX). In some embodiments, the probes (e.g., molecular beacons) provided herein comprise a 3′ modification (e.g., 3IABkFQ).

TABLE 1
HPIC ASSAY COMPONENTS

| | | SEQ |
|---|---|---|
| | | ID |
| Name | Sequence | NO |
| IC Primer | AAAGATCCCTA*A | 3 |
| HpIC1 MB2 | AAAGATC+CCTAA+CA+CCA+CTTAG+GG+A | 4 |
| IC Template | AGATCCCTAACACCACTTAGGGATCTaaaa | 10 |
| (HpIC DNA26) | | |

*indicates phosphorothioated DNA bases (e.g. A*, G*, C*, T*)
*denotes phosphorothioate linkage between adjacent bases
+indicates locked nucleic acids (e.g. +A, +G, +C, +T)

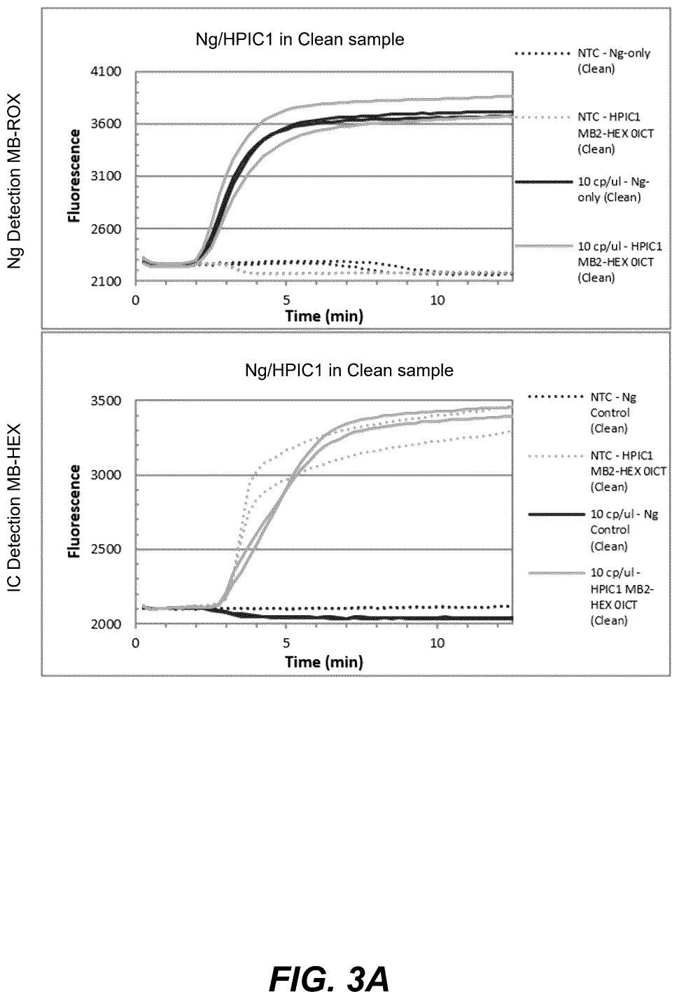

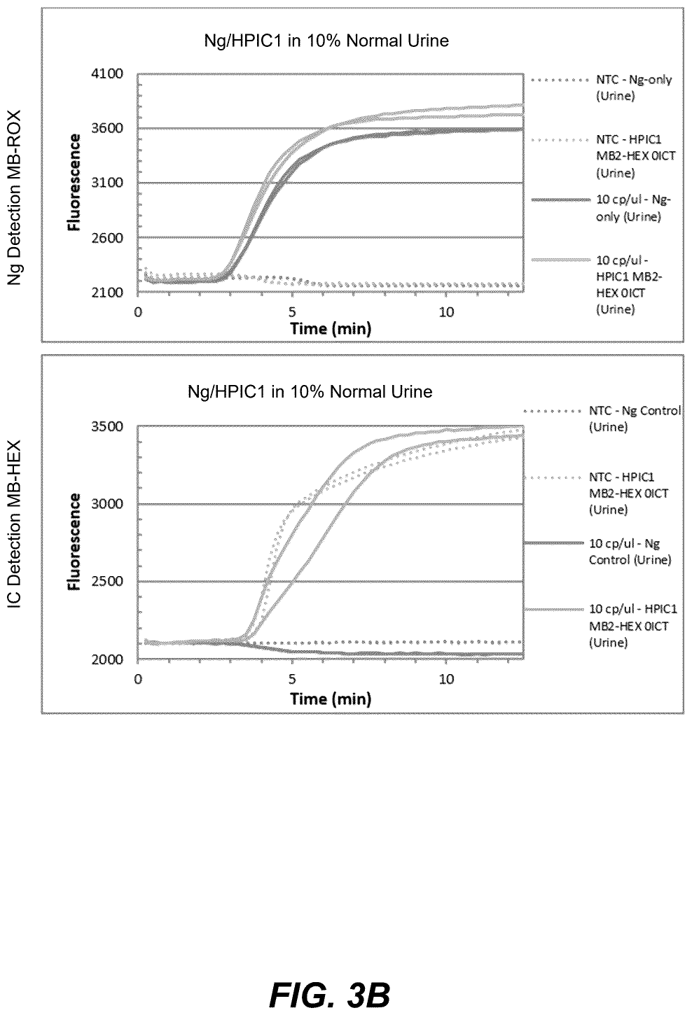

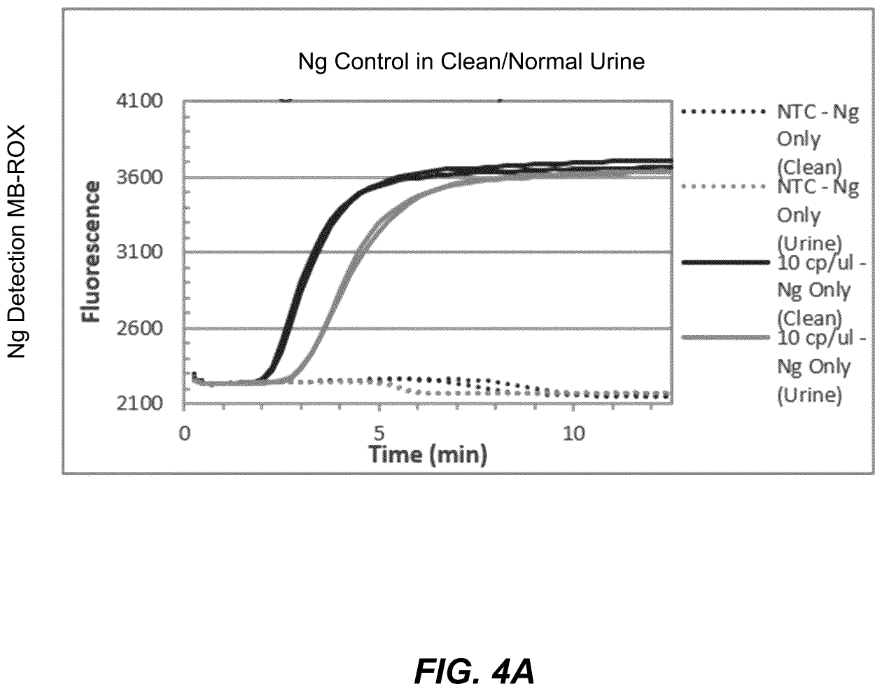

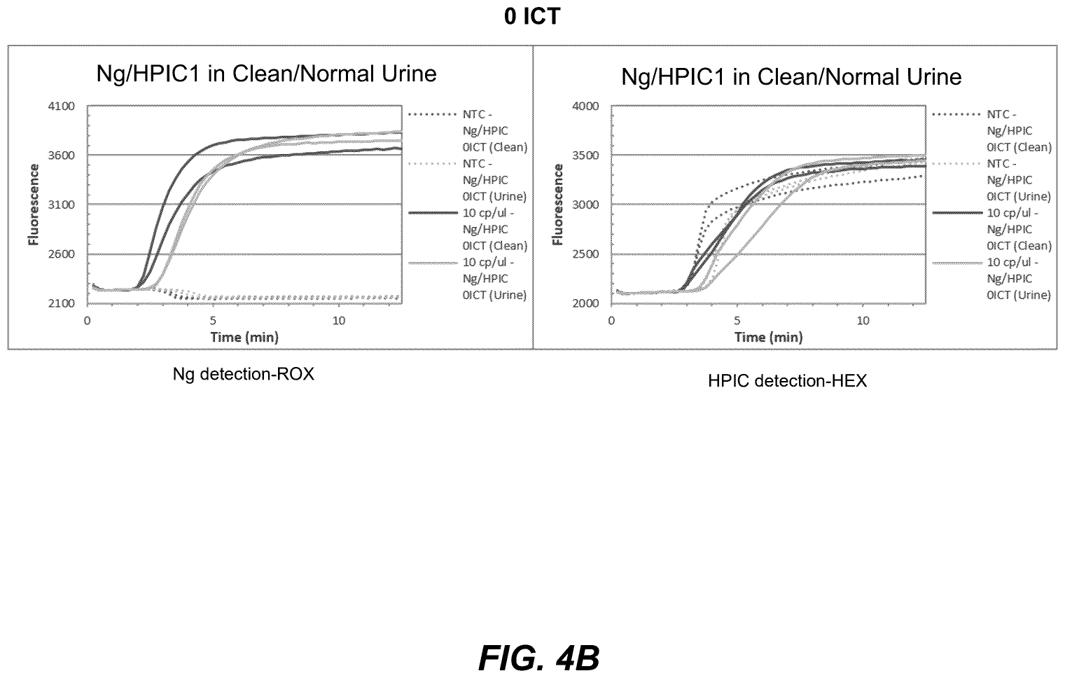

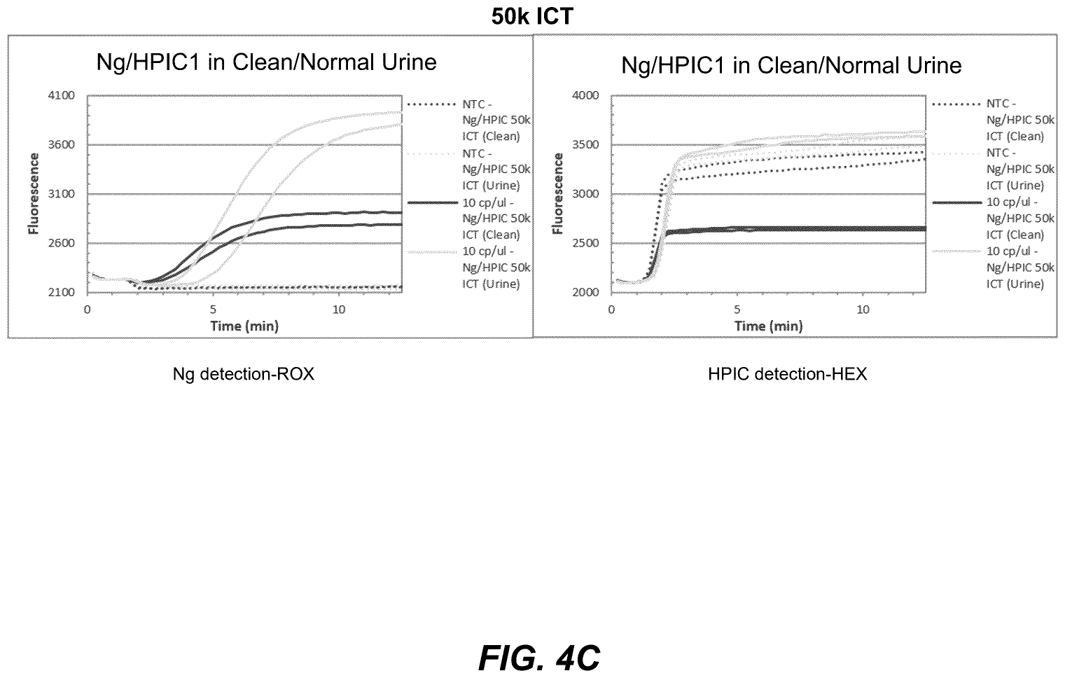

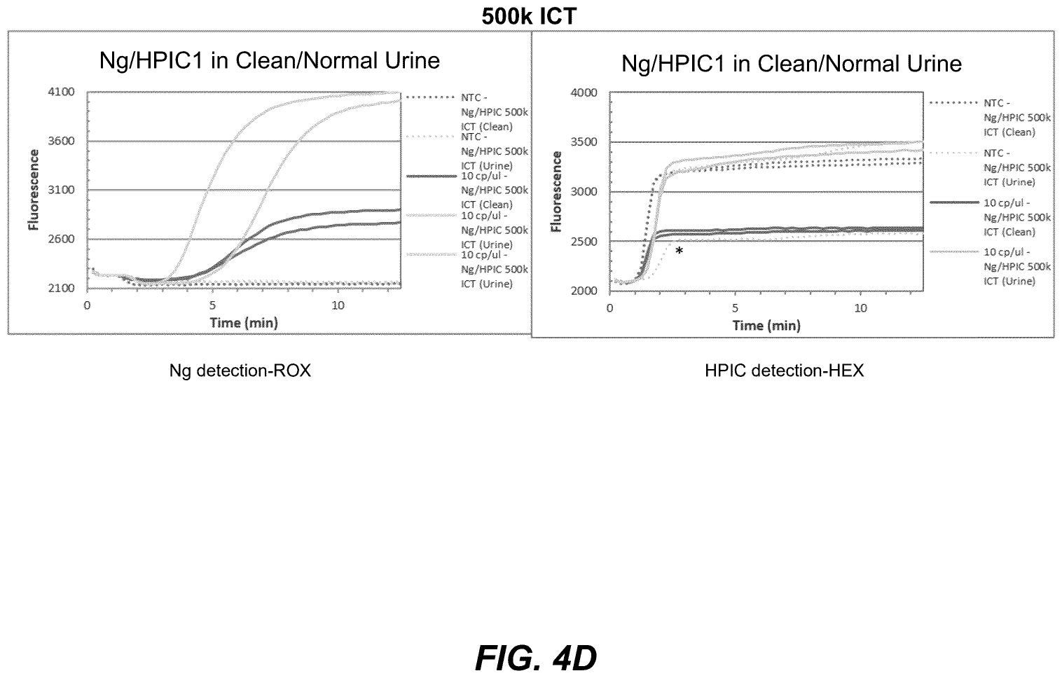

The performance of a duplex system of hairpin IC (400 nM) and target Neisseria gonorrhoeae (Ng) detection was evaluated in clean samples versus 10% normal urine samples and with HPIC template titration. FIG. 3A-FIG. 3B show data related to target (Ng) (ROX) detection and Hairpin IC detection (HEX) in clean samples (FIG. 3A) as compared to samples comprising 10% normal urine (FIG. 3B). FIG. 4A shows a Ng only reaction and FIGS. 4B-FIG. 4D depict reactions with increasing levels of IC template (ICT): 0 ICT (FIG. 4B), 50 k ICT (FIG. 4C), and 500 k ICT (FIG. 4D) in clean samples and 10% urine samples. These results show that the HPIC (e.g., MB2-HEX) operates as both a beacon for detection and as a template. The consequence of including additional IC template in mixes was examined, and it was found that increasing IC template can make the IC too strong to have accurate detection and, without being bound by any particular theory, may compete with target (e.g., Ng) signal.

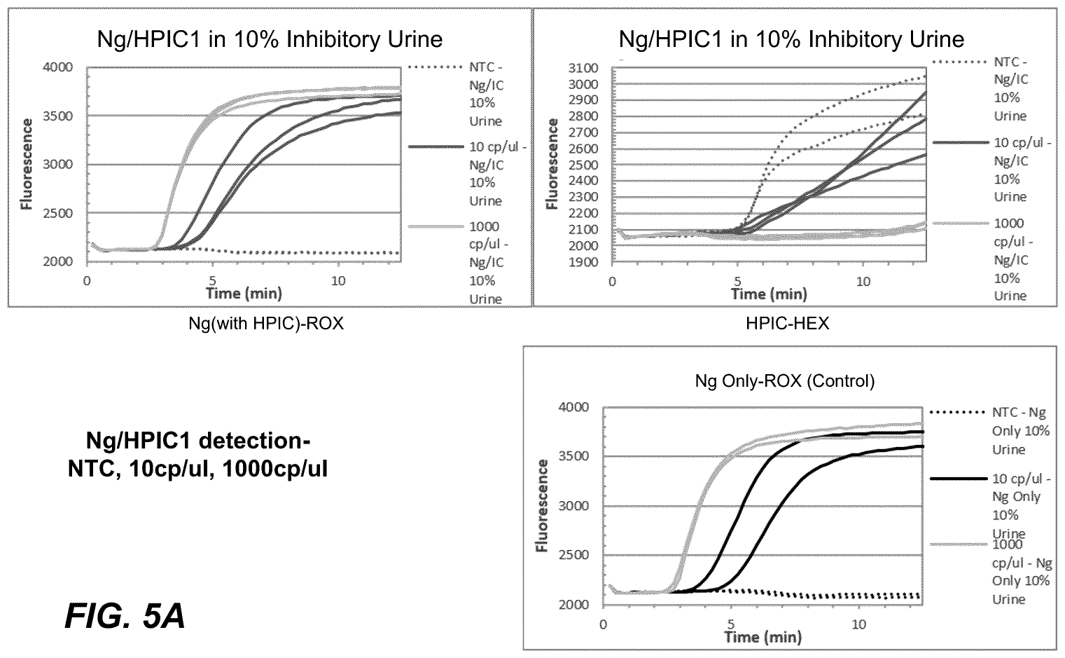

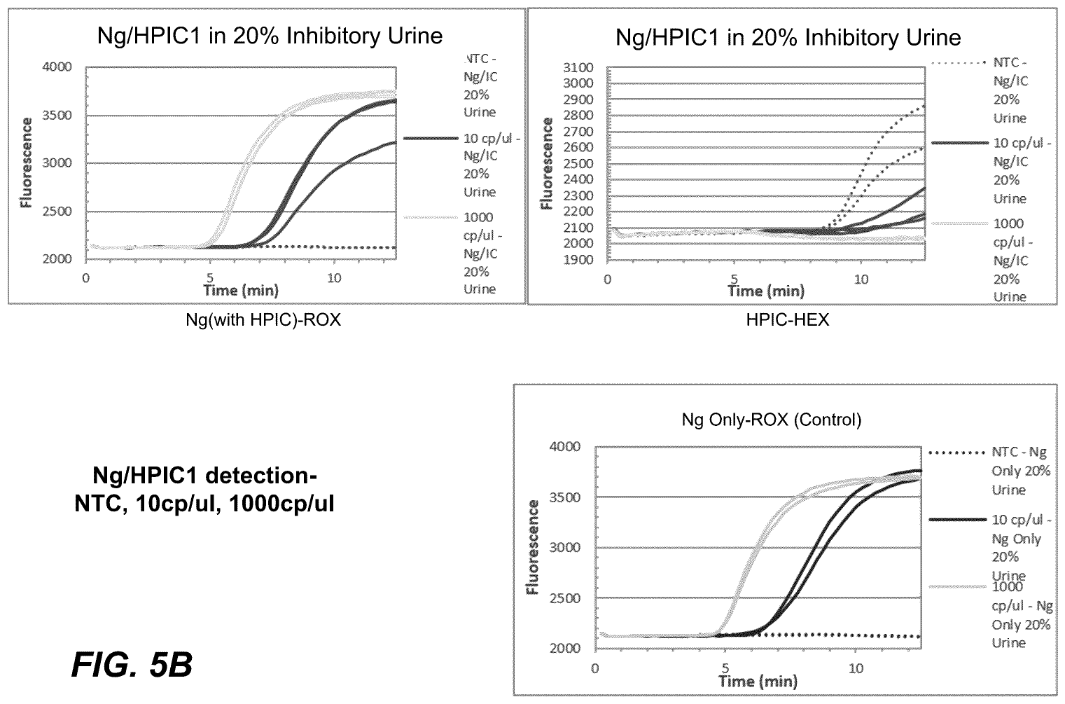

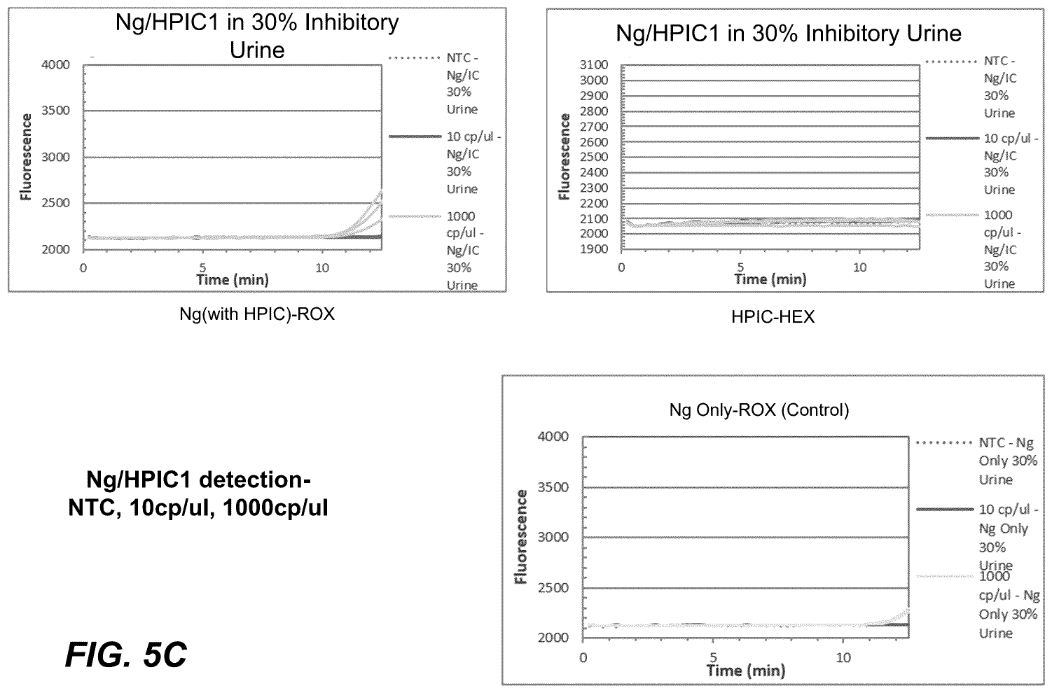

Next, the performance of a duplex system of hairpin IC (400 nM) and target (Ng) detection was evaluated in samples with increasing levels of inhibitory urine: 10% (FIG. 5A), 20% (FIG. 5B), and 30% (FIG. 5C). The MB hairpin IC was found to be detectably amplified in samples comprising 10% or 20% Inhibitory Urine.

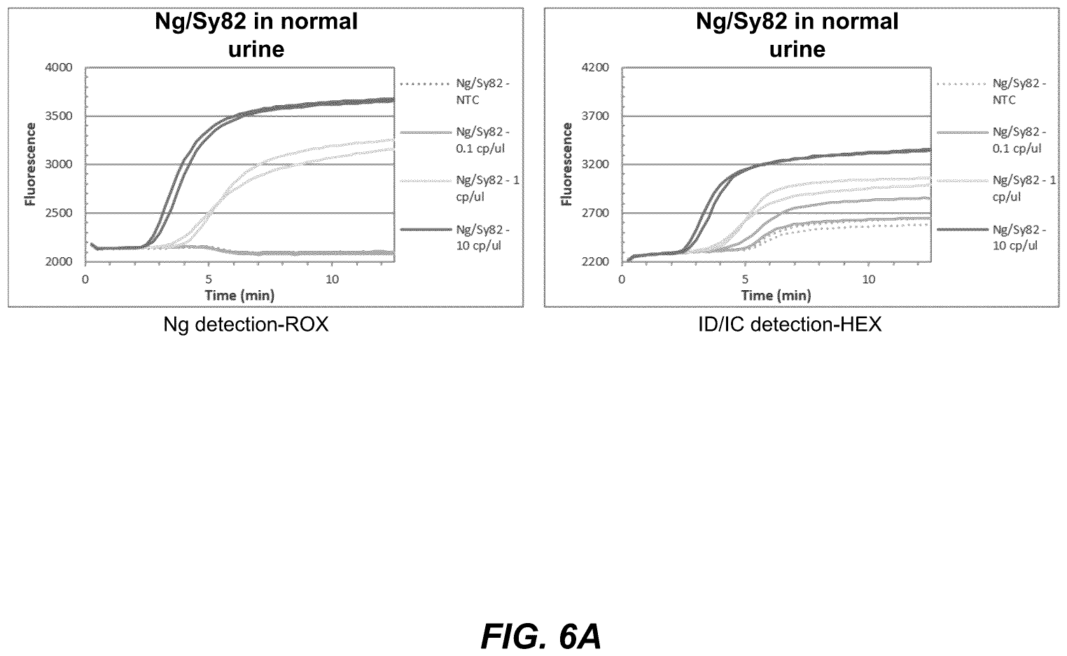

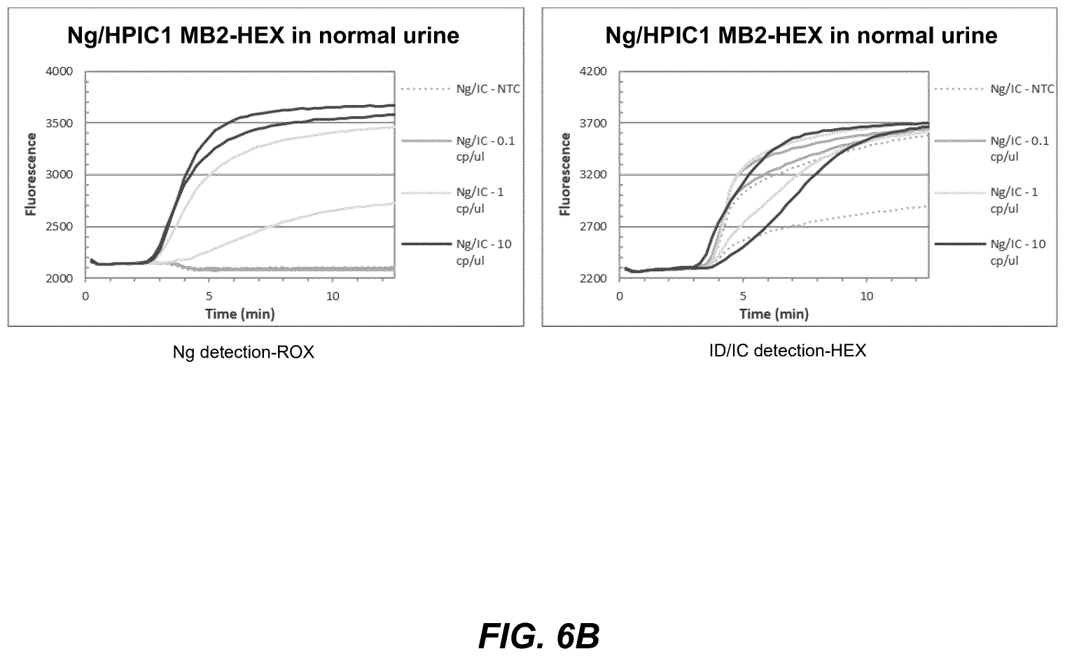

FIG. 6A depicts data related to Ng detection in normal urine in the absence of an internal control. Ng ROX probe was used for target detection and Syto 82 intercalating dye was used for both specific and nonspecific product detections. FIG. 6B depicts data related to Ng/Hairpin IC detection in normal urine where the hairpin IC probe (HPIC) is detected via HEX fluorophore and the target (Ng) is detected via ROX fluorophore.

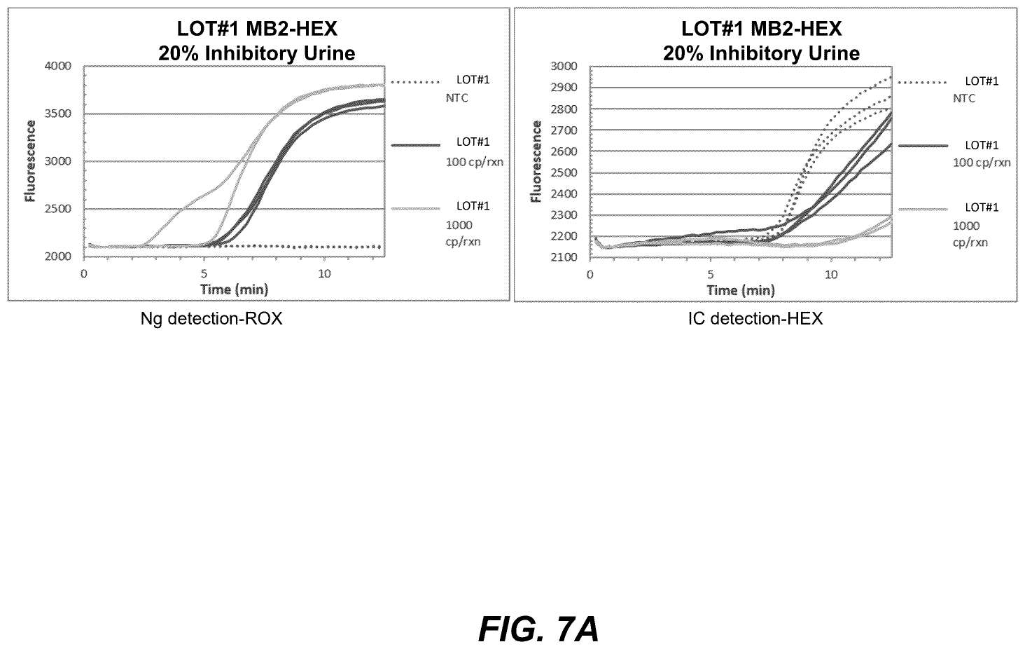

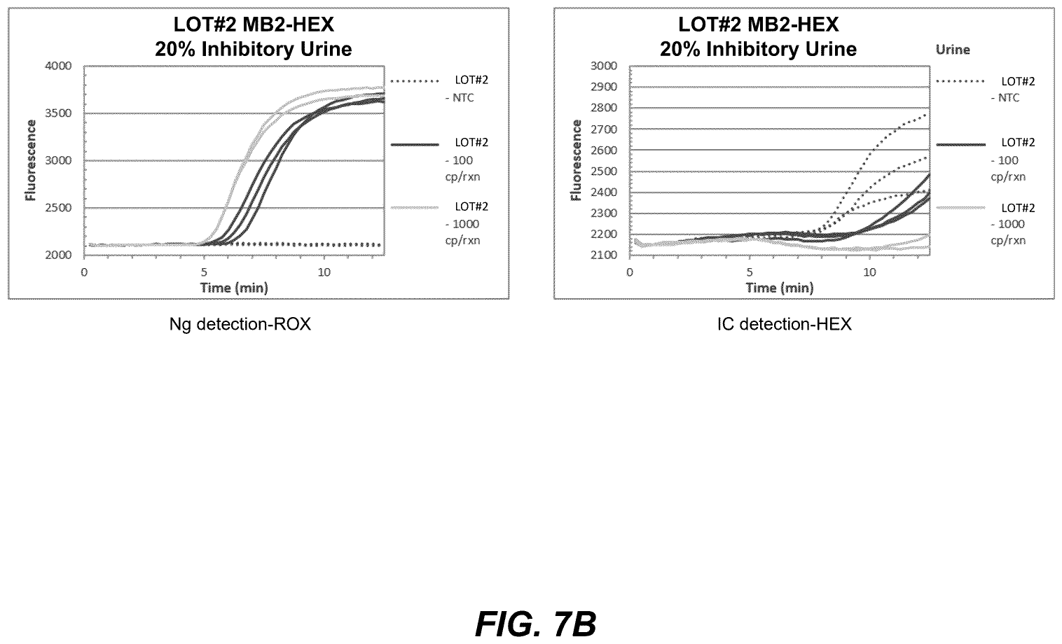

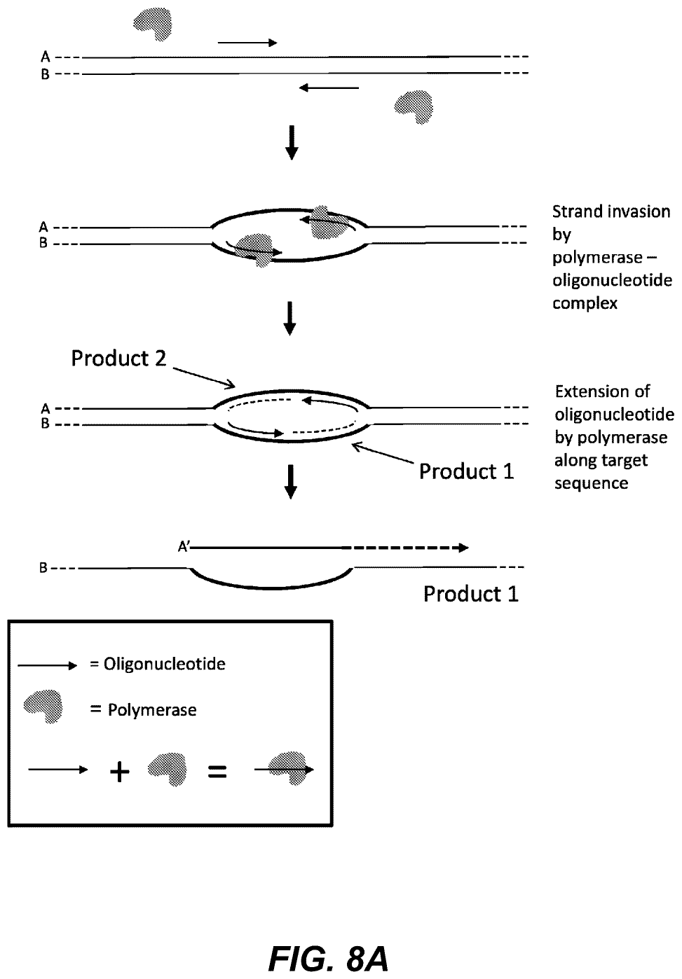

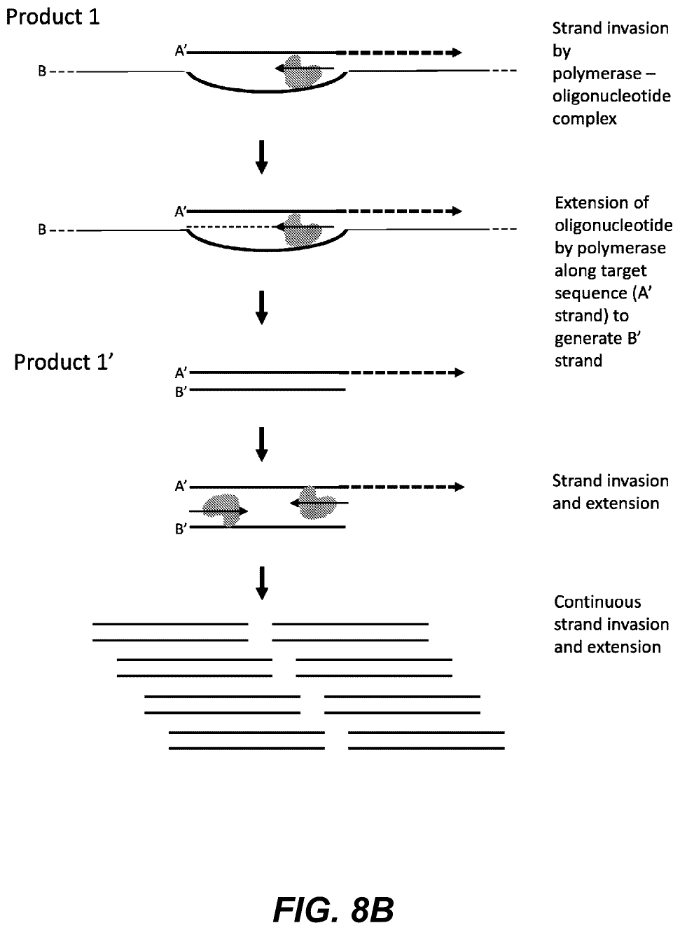

The MB hairpin IC employed in this Example also shows similar results across different lots. FIG. 7A-FIG. 7B depict data showing a comparison of target (Ng) (ROX) detection and Hairpin IC detection (HEX) in samples comprising 20% inhibitory urine and Hairpin IC from different lots: 9.14 (FIG. 7A) and 10.12 (FIG. 7B).

In summary, this Example demonstrates that the hairpin IC MB can function as both an IC beacon and as a template, without the need to add additional IC template. The addition of the hairpin IC MB did not interfere negatively with target (Ng) detection. The hairpin IC can be fine-tuned to track target amplification for matrix inhibition. The IC reactions performed well in samples containing interfering substances (e.g., in 10% normal urine, 10% inhibitory urine, and 20% inhibitory urine matrix). The IC signal can increase as target (Ng) decreases. In some cases, there is an about 1 minute difference between Ng signal Td and IC signal Td in 10% inhibitory urine and an about 2 minutes difference in 20% inhibitory urine. In samples with 10% inhibitory urine and 10 cp/ul of target sequence, the Td was about 4 minutes for the target and the IC signal appeared at about 5 minutes. In samples with 20% inhibitory urine and 10 cp/ul of target, the Td was about 7 minutes for the target, and the IC signal appeared at about 9 minutes.

### Example 2

### Hairpin Internal Control (HPIC) Molecular Beacon Modification

This example demonstrates an exemplary modification of a signal-generating oligonucleotide (e.g., molecular beacon) as described herein. For ease of amplification and detection, the product hybridization melting temperature (Tm) can be designed to be greater than or equal to the product hairpin Tm. A signal-generating oligonucleotide (e.g., molecular beacon) modified with LNAs in the spacer region can be used for IC product detection.

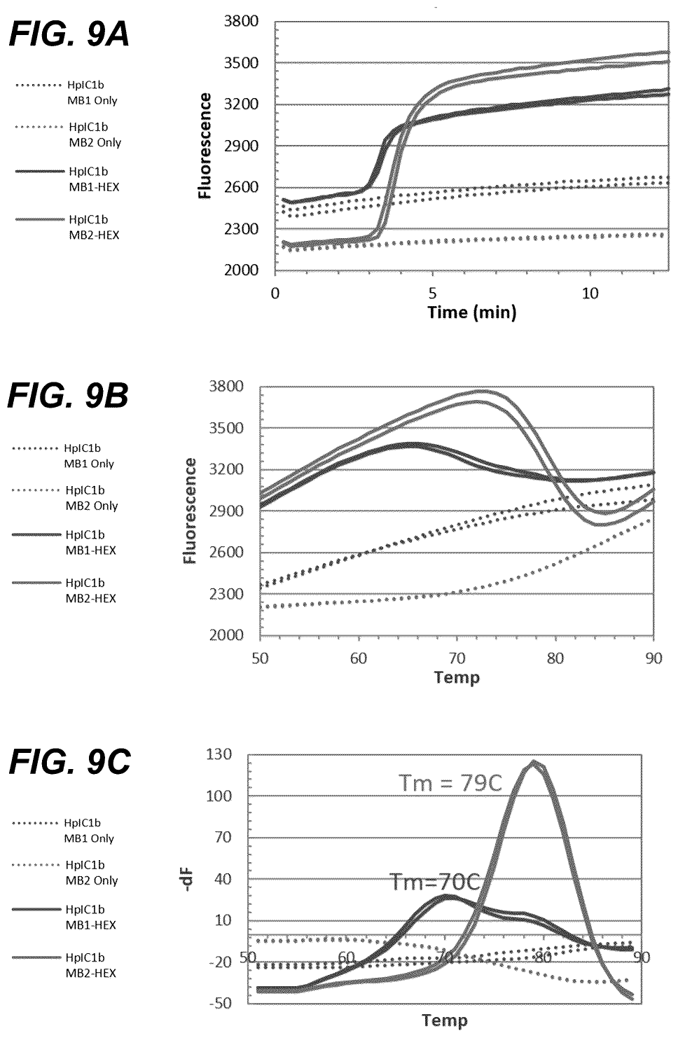

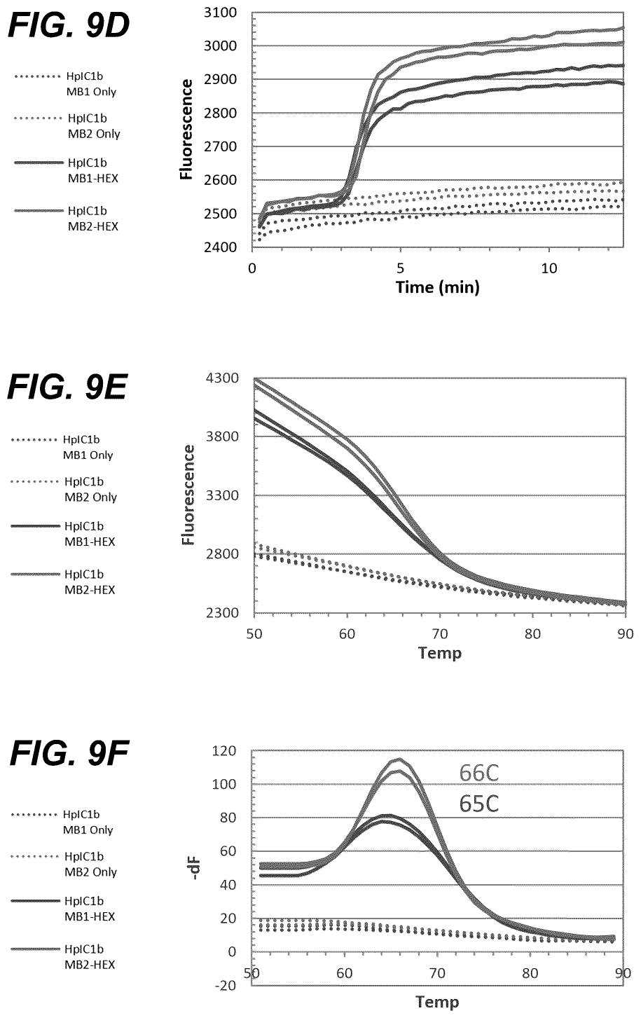

In this example, a hairpin-shaped internal control target was amplified in APA reaction for 10 minutes with simultaneous detection by molecular beacon (FIG. 9A) and intercalating dye (FIG. 9D). After the reaction, the temperature of the reactions was immediately ramped up from assay temperature to 90° C. for melting curve analysis (FIG. 9B and FIG. 9E) and melt derivatives assessment (FIG. 9C and FIG. 9F). The sequences of the two molecular beacons (50 nM) and internal control primer (500 nM) are shown in Table 2. The two molecular beacon designs (HpIC1b MB1 and HpIC1b MB2) contain the same fluorophore and quencher pair and have the same sequence. They differ only in the locations of LNA modification on the beacon. The resulting melting temperature difference for the two beacons MB1 (Red curves, upper right) and MB2 (Green curves, lower right) is 9° C. In some embodiments, the probes (e.g., molecular beacons) provided herein comprise a 5′ modification (e.g., 5HEX). In some embodiments, the probes (e.g., molecular beacons) provided herein comprise a 3′ modification (e.g., 3IABkFQ).

TABLE 2
IC ASSAY COMPONENTS

| | | SEQ |
|---|---|---|
| | | ID |
| Name | Sequence | NO |
| HpIC1b MB1 | AAAGTACCCTAA+CA+CCA+CT+TAG+GG+T | 8 |
| HpIC1b MB2 | AAAGTACC+CTAA+CA+CCACT+TAG+GG+T | 11 |
| IC Primer | AAAGTACCCTA*A | 9 |

*indicates phosphorothioated DNA bases (e.g. A*, G*, C*, T*)
*denotes phosphorothioate linkage between adjacent bases
+indicates locked nucleic acids (e.g. +A, +G, +C, +T)

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for monitoring an amplification reaction, comprising:
providing:
a quality control template comprising:
a 5′ subdomain;
a 3′ subdomain; and
a loop domain situated between the 5′ subdomain and the 3′ subdomain, and wherein intramolecular nucleotide base pairing between the 5′ subdomain and the 3′ subdomain are capable of forming a paired stem domain; and
a quality control primer capable of hybridizing to at least a portion of the 3′ subdomain of the quality control template;
subjecting the quality control template and the quality control primer to an amplification reaction capable of generating a first quality control product; and
detecting the first quality control product.

2. The method of claim 1, wherein the amplification reaction is conducted in an amplification reaction mixture under an amplification condition, optionally the amplification condition is an isothermal amplification condition.

3. The method of any one of claims 1-2, wherein subjecting the quality control template and the quality control primer to an amplification reaction capable of generating a first quality control product comprises:
amplifying the quality control template with the quality control primer in the amplification reaction mixture under the amplification condition, thereby generating the first quality control product.

4. The method of any one of claims 1-3, wherein the amplification reaction comprises a reverse transcription reaction.

5. The method of any one of claims 1-4, wherein the method further comprises:
providing an enzyme having a polymerase activity, optionally the enzyme having a polymerase activity is an enzyme having a hyperthermophile polymerase activity, and further optionally the enzyme having a hyperthermophile polymerase activity has a reverse transcriptase activity; and/or
providing a reverse transcriptase.

6. The method of any one of claims 1-5, wherein the amplification reaction comprises:
contacting the quality control primer with the quality control template for hybridization, and
extending the quality control primer hybridized to the quality control template with an enzyme having a polymerase activity, thereby generating a first quality control product.

7. The method of any one of claims 1-6, wherein the amplification reaction comprises:
contacting the quality control primer with the first quality control product for hybridization, and
extending the quality control primer hybridized to the first quality control product with an enzyme having a polymerase activity, thereby generating a second quality control product.

8. The method of any one of claims 1-7, wherein the amplification reaction comprises:
contacting the quality control primer with the second quality control product for hybridization, and
extending the quality control primer hybridized to the second quality control product with an enzyme having a polymerase activity, thereby generating a first quality control product.

9. The method of any one of claims 1-8, wherein:
the first quality control product and second quality control product comprise a 5′ subdomain and the 3′ subdomain capable of forming a paired stem domain;
the first quality control product and second quality control product have the same stem domain; and/or
the first quality control product and the second first quality control product comprise a loop domain complementary to each other.

10. The method of any one of claims 1-9, wherein the amplification reaction comprises linear and/or exponential amplification the first quality control product and the second quality control product.

11. The method of any one of claims 1-10, wherein the 5′ subdomain of the quality control template comprises the sequence of at least a portion of the quality control primer.

12. The method of any one of claims 1-11, wherein the first quality control product and the second quality control product are both capable of forming a hairpin structure.

13. The method of any one of claims 1-12, wherein:
the quality control template comprises a 5′ terminal domain situated 5′ of the 5′ subdomain, and/or
the quality control template comprises a 3′ terminal domain situated 3′ of the 3′ subdomain.

14. The method of any one of claims 1-13, wherein the 5′ terminal domain of the quality control template comprises at least a portion of the sequence of the quality control primer, optionally the combined sequence of the 5′ terminal domain and the 5′ subdomain comprises the entire sequence of the quality control primer.

15. The method of any one of claims 1-14, wherein detecting the first quality control product comprises detecting the first quality control product with a signal-generating oligonucleotide, optionally the signal-generating oligonucleotide is capable of hybridizing to the first quality control product.

16. The method of any one of claims 1-15, wherein the detecting comprises contacting the first quality control product with the signal-generating oligonucleotide for hybridization.

17. The method of any one of claims 1-16, wherein the signal-generating oligonucleotide comprises a quencher, a label, or both, optionally the label comprises a quenchable label, further optionally the quenchable label is a fluorophore.

18. The method of any one of claims 1-17, wherein the signal-generating oligonucleotide comprises a quencher, optionally the quencher is capable of quenching the label.

19. The method of any one of claims 1-18, wherein the detecting comprises contacting the first quality control product with the signal-generating oligonucleotide for hybridization.

20. The method of any one of claims 1-19, wherein:
the label is capable of generating a signal upon the signal-generating oligonucleotide hybridizing the first quality control product; and/or
upon the signal-generating oligonucleotide hybridizing the first quality control product, the label generates a signal,
optionally the signal is fluorescence.

21. The method of any one of claims 1-20, wherein detecting the first quality control product comprises detecting a signal generated by the label of the signal-generating oligonucleotide, optionally the label is a fluorophore and the signal is fluorescence.

22. The method of any one of claims 1-21, wherein the detecting comprises detecting the signal of the label before the amplification reaction, during the amplification reaction, after the amplification reaction, or any combination thereof.

23. The method of any one of claims 1-22, wherein the method further comprises:
providing a signal-generating oligonucleotide;
subjecting the signal-generating oligonucleotide to the amplification reaction; and
detecting the first quality control product with the signal-generating oligonucleotide.

24. The method of any one of claims 1-23, wherein the quality control template is a signal-generating oligonucleotide.

25. The method of any one of claims 1-24, wherein the quality control template is (i) a template for the synthesis of the first quality control product, and (ii) a means of detecting the first quality control product.

26. The method of any one of claims 1-25, wherein the signal-generating oligonucleotide is capable of (i) detecting the first quality control product and (ii) being a template for the quality control primer-driven synthesis of the first quality control product.

27. The method of any one of claims 1-26, wherein (i) the quality control template, (ii) the quality control primer, and (iii) the signal-generating oligonucleotide, are present in the same molecule.

28. The method of any one of claims 1-27, wherein the 5′ terminal domain of the quality control template comprises:
one or more RNA nucleotides; and/or
the sequence of at least a portion of the quality control primer.

29. The method of any one of claims 1-28, wherein:
the quality control template does not comprise a 3′ terminal domain; and/or
the 3′ end of the quality control template is complementary to the 5′ end of the 5′ subdomain of the quality control template.

30. The method of any one of claims 1-29, wherein a reverse transcriptase is capable using the one or more RNA nucleotides of the 5′ terminal domain of the quality control template as a template to extend the 3′ end of the quality control template, thereby generating an extended quality control template.

31. The method of any one of claims 1-30, wherein the 3′ end of the extended quality control template comprises a sequence complementary to at least a portion of the quality control primer.

32. The method of any one of claims 1-31, wherein the amplification reaction comprises contacting a reverse transcriptase with the quality control template, thereby generating an extended quality control template, optionally the extended quality control template comprises cDNA.

33. The method of any one of claims 1-32, wherein the amplification reaction comprises:
contacting the quality control primer with the 3′ end of the extended quality control template for hybridization, and
extending the quality control primer hybridized to the 3′ end of the extended quality control template with a reverse transcriptase and/or an enzyme having a polymerase activity, thereby generating a first quality control product.

34. The method of any one of claims 1-33, wherein the quality control template is a signal-generating oligonucleotide, wherein the signal-generating oligonucleotide comprises a label, and wherein the loop domain comprises one or more RNA nucleotides, optionally the label comprises a quenchable label, further optionally the quenchable label is a fluorophore.

35. The method of any one of claims 1-34, wherein the signal-generating oligonucleotide comprises a quencher, optionally:
the label is situated in the 3′ terminal domain and the quencher is situated in the 5′ terminal domain, and/or
the label is situated in the 5′ terminal domain and the quencher is situated in the 3′ terminal domain.

36. The method of any one of claims 1-35, wherein the amplification reaction comprises:
contacting the quality control primer with the quality control template for hybridization, and
extending the quality control primer hybridized to the quality control template with a reverse transcriptase, thereby generating a first quality control product, optionally the reverse transcriptase comprises RNaseH activity.

37. The method of any one of claims 1-36, wherein the reverse transcriptase cleaves the quality control template at the one or more RNA nucleotides during the generation of the first quality control product, thereby generating a first cleavage product comprising a label and a second cleavage product.

38. The method of any one of claims 1-37, wherein detecting the first quality control product comprises detecting a signal generated by the first cleavage product comprising a label, optionally the label is a fluorophore and the signal is fluorescence.

39. The method of any one of claims 1-38, wherein the method further comprises:
providing a supplemental quality control primer; and
subjecting the supplemental quality control primer to the amplification reaction.

40. The method of any one of claims 1-39, wherein:
the signal-generating oligonucleotide is about 10 nucleotides to about 100 nucleotides in length;
the quality control template is about 10 nucleotides to about 100 nucleotides in length;
the quality control primer and/or the supplemental quality control primer is about 5 nucleotides to about 25 nucleotides in length; and/or
the 5′ subdomain, the 3′ subdomain, the loop domain, the 5′ terminal domain, and/or the 3′ terminal domain is about 1 nucleotide to about 25 nucleotides in length.

41. The method of any one of claims 1-40, wherein the signal-generating oligonucleotide, the quality control template, and/or the quality control primer comprises one or more phosphorothioate linkages and/or one or more locked nucleic acids.

42. The method of any one of claims 1-41, wherein the signal-generating oligonucleotide is a TaqMan detection probe oligonucleotide, a molecular beacon detection probe oligonucleotide, or a molecular torch detection probe oligonucleotide.

43. The method of any one of claims 1-42, wherein the signal-generating oligonucleotide comprises one or more locked nucleic acids (LNAs), optionally the one or more LNAs are situated within the loop domain and/or stem domain, further optionally the one or more LNAs enhance the detectability of the first quality control product.

44. The method of any one of claims 1-43, wherein the signal-generating oligonucleotide is configured such that the melting temperature (Tm) of first quality control product/signal-generating oligonucleotide duplex is equal to or greater than the melting temperature (Tm) of the paired stem domain of the signal-generating oligonucleotide, optionally configured via one or more LNAs situated in the loop domain and/or stem domain.

45. The method of any one of claims 1-44, wherein the signal-generating oligonucleotide does not comprise a dye capable of quenching the label.

46. The method of any one of claims 1-45, wherein the signal-generating oligonucleotide does not comprise a moiety capable of quenching the label other than the nucleotides of said signal-generating oligonucleotide.

47. The method of any one of claims 1-46,
wherein the 5′ terminal domain of the quality control template and/or the signal-generating oligonucleotide comprises the label,
wherein the 5′ terminal domain and/or the 5′ subdomain of the quality control template and/or the signal-generating oligonucleotide comprises one or more cytosine bases,
wherein the 3′ terminal domain and/or the 3′ subdomain of the quality control template and/or the signal-generating oligonucleotide comprises one or more guanine bases and/or adenine bases,
and optionally the one or more guanine bases and/or adenine bases are capable of quenching the label upon the quality control template and/or the signal-generating oligonucleotide forming a hairpin structure.

48. The method of any one of claims 1-47,
wherein the 3′ terminal domain of the quality control template and/or the signal-generating oligonucleotide comprises the label,
wherein the 3′ terminal domain and/or the 3′ subdomain of the quality control template and/or the signal-generating oligonucleotide comprises one or more cytosine bases,
wherein the 5′ terminal domain and/or the 5′ subdomain of the quality control template and/or the signal-generating oligonucleotide comprises one or more guanine bases and/or adenine bases,
and optionally the one or more guanine bases and/or adenine bases are capable of quenching the label upon the quality control template and/or the signal-generating oligonucleotide forming a hairpin structure.

49. The method of any one of claims 1-48, wherein detecting the first quality control product comprises detecting a reduction in the amount of signal generated by a label of the quality control primer, optionally the label is a fluorophore and the signal is fluorescence.

50. The method of any one of claims 1-49, wherein the generation of the first quality control product and second quality control product is correlated with a decline in the signal detected.

51. The method of any one of claims 1-50, wherein the 5′ end of the quality control primer comprises a label, and wherein the quality control primer comprises one or more pyrimidine bases adjacent to the label, optionally one or more cytosine bases and/or thymine bases adjacent to the label.

52. The method of any one of claims 1-51, wherein the 3′ terminal domain and/or the 3′ subdomain of the quality control template, first quality control product, and/or the second quality control product comprises one or more guanine bases and/or adenine bases.

53. The method of any one of claims 1-52, wherein:
upon the quality control primer binding the quality control template and/or second quality control product and being extended with an enzyme having a polymerase activity to generate a first quality control product, the one or more guanine bases and/or adenine bases present in the 3′ terminal domain and/or the 3′ subdomain of the first quality control product are capable of quenching the label upon first quality control product forming a hairpin structure; and/or
upon the quality control primer binding the first quality control product and being extended with an enzyme having a polymerase activity to generate a second quality control product, the one or more guanine bases and/or adenine bases present in the 3′ terminal domain and/or the 3′ subdomain of the second quality control product are capable of quenching the label upon second quality control product forming a hairpin structure.

54. The method of any one of claims 1-53, wherein detecting the first quality control product comprises contacting the first quality control product with a fluorescence dye.

55. The method of any one of claims 1-54, wherein providing the quality control primer, the quality control template, and/or the signal-generating oligonucleotide comprises providing a reagent composition comprising the quality control primer, the quality control template, and/or the signal-generating oligonucleotide.

56. The method of any one of claims 1-55, wherein subjecting the quality control primer, the quality control template, and/or the signal-generating oligonucleotide to an amplification reaction comprises contacting the reagent composition with a treated sample to generate the amplification reaction mixture.

57. The method of any one of claims 1-56, wherein the method further comprises detecting a target nucleic acid sequence in a sample.

58. The method of any one of claims 1-57, wherein the method comprises:
subjecting the target nucleic acid sequence to an amplification reaction capable of generating a nucleic acid amplification product, and
detecting the nucleic acid amplification product with a target signal-generating oligonucleotide, wherein the target signal-generating oligonucleotide is capable of hybridizing to the nucleic acid amplification product.

59. The method of any one of claims 1-58, wherein subjecting the target nucleic acid sequence to an amplification reaction capable of generating a nucleic acid amplification product comprises:
amplifying the target nucleic acid sequence in the amplification reaction mixture under the amplification condition, thereby generating a nucleic acid amplification product.

60. The method of any one of claims 1-59, comprising:
contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence; and
contacting the reagent composition with the treated sample to generate the amplification reaction mixture, wherein the reagent composition comprises one or more amplification reagents.

61. The method of any one of claims 1-60, wherein the one or more amplification reagents comprise:
a reverse transcriptase;
an enzyme having a hyperthermophile polymerase activity, optionally the enzyme having a hyperthermophile polymerase activity has a reverse transcriptase activity
a forward primer;
a reverse primer;
a reverse transcription primer; and/or
dNTPS.

62. The method of any one of claims 1-61, wherein the sample nucleic acids comprise a nucleic acid comprising the target nucleic acid sequence.

63. The method of any one of claims 1-62, wherein amplifying the target nucleic acid sequence comprises:
amplifying a target nucleic acid sequence comprising a first strand and a second strand complementary to each other in an isothermal amplification condition, wherein the amplifying comprises contacting a nucleic acid comprising the target nucleic acid sequence with:
i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and
ii) an enzyme having a hyperthermophile polymerase activity, thereby generating the nucleic acid amplification product.

64. The method of any one of claims 1-63, wherein the nucleic acid is:
a double-stranded DNA; and/or
a product of reverse transcription reaction, optionally the nucleic acid is a product of reverse transcription reaction generated from sample ribonucleic acids, further optionally the amplifying comprises generating the nucleic acid by a reverse transcription reaction.

65. The method of any one of claims 1-64, wherein:
the amplification reaction is performed for a period of about 5 minutes to about 60 minutes;
amplifying the quality control template comprises generating the first quality control product and/or the second quality control product at detectable levels within about 20 minutes, about 15 minutes, or about 10 minutes;
amplifying the target nucleic acid sequence comprises generating the nucleic acid amplification product at detectable levels within about 20 minutes, about 15 minutes, or about 10 minutes; and/or
the detecting is performed in less than about 20 minutes, less than about 15 minutes, less than about 10 minutes, or less than about 5 minutes, from the time the reagent composition is contacted with the treated sample.

66. The method of any one of claims 1-65, wherein:
the lysis buffer comprises one or more of magnesium sulfate, ammonium sulfate, EDTA, and EGTA; and/or
the pH of the lysis buffer is about 1.0 to about 10.0, optionally the pH of the lysis buffer is about 2.2.

67. The method of any one of claims 1-66, wherein the sample nucleic acids comprise sample ribonucleic acids and/or sample deoxyribonucleic acids, optionally the sample nucleic acids comprise cellular RNA, mRNA, microRNA, bacterial RNA, viral RNA, or a combination thereof.

68. The method of any one of claims 1-67, wherein the reagent composition is lyophilized, heat-dried, and/or comprises one or more additives, wherein the one or more additives comprise:
Tween 20, Triton X-100, and/or tween 80;
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof, and/or
a polymer, optionally the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof,
and optionally contacting the reagent composition with the treated sample comprises dissolving the reagent composition in the treated sample.

69. The method of any one of claims 1-68, wherein the one or more lytic reagents comprise:
about 0.001% (w/v) to about 1.0 (w/v) of the treated sample, optionally about 0.2% (w/v) of the treated sample; and/or
a detergent, optionally the detergent comprises one or more of a cationic surfactant, an anionic surfactant, a non-ionic surfactant, and an amphoteric surfactant.

70. The method of any one of claims 1-69, wherein the method:
is performed in a single reaction vessel;
does not comprise using any enzymes other than the reverse transcriptase and the enzyme having a hyperthermophile polymerase activity;
does not comprise using any enzyme other than the enzyme having a hyperthermophile polymerase activity;
does not comprise heat denaturing and/or enzymatic denaturing the nucleic acid and/or quality control template during the amplification step; and/or
does not comprise contacting the nucleic acid and/or quality control template with a signal-stranded DNA binding protein.

71. The method of any one of claims 1-70, wherein:
the target nucleic acid sequence comprises a length of no longer than about 20 nucleotides to no longer than about 90 nucleotides, optionally the target nucleic acid sequence comprises a length of about 30 nucleotides;
the forward primer, the reverse primer, and/or the reverse transcription primer is about 8 to 16 bases long;
the nucleic acid amplification product is about 20 to 40 bases long; and/or
the spacer sequence comprises a portion of the target nucleic acid sequence, optionally the spacer sequence is 1 to 10 bases long.

72. The method of any one of claims 1-71, wherein the isothermal amplification condition comprises a constant temperature of about 30° C. to about 72° C., further optionally about 55° C. to about 75° C., optionally about 56° C. to about 67° C.

73. The method of any one of claims 1-72, wherein the amplifying is performed:
for a period of about 5 minutes to about 60 minutes, optionally the amplifying is performed for a period of about 15 minutes; and/or
in helicase-free, signal-stranded binding protein-free, cleavage agent-free, and recombinase-free, isothermal amplification conditions.

74. The method of any one of claims 1-73, wherein the enzyme having a hyperthermophile polymerase activity has an amino acid sequence that is at least about 90% identical to the amino acid sequence of SEQ ID NO: 1 or a functional fragment thereof, optionally the enzyme having a hyperthermophile polymerase activity has an amino acid sequence that is at least about 95% identical to the amino acid sequence of SEQ ID NO: 1, further optionally the enzyme having a hyperthermophile polymerase activity is a polymerase comprising the amino acid sequence of SEQ ID NO: 1, optionally the enzyme having a hyperthermophile polymerase activity has low or no exonuclease activity.

75. The method of any one of claims 1-74, wherein the sample ribonucleic acids are contacted with the reverse transcriptase and the enzyme having a hyperthermophile polymerase activity simultaneously, optionally the sample ribonucleic acids are contacted with the reverse transcriptase, the enzyme having a hyperthermophile polymerase activity, and the forward and reverse primers simultaneously, further optionally the sample ribonucleic acids are contacted with the reverse transcriptase, the enzyme having a hyperthermophile polymerase activity, the forward primer, the reverse primer, and the reverse transcription primer simultaneously.

76. The method of any one of claims 1-75, wherein:
the biological entities comprise one or more of prokaryotic cells, eukaryotic cells, viral particles, exosomes, protoplasts, and microvesicles;
the biological entities comprise a virus, a bacteria, a fungi, a protozoa, portions thereof, or any combination thereof, and/or
the target nucleic acid sequence is a nucleic acid sequence of a virus, bacteria, fungi, or protozoa, optionally the sample nucleic acids are derived from a virus, bacteria, fungi, or protozoa.

77. The method of any one of claims 1-76, wherein the sample is a biological sample or an environmental sample,
wherein the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof; and/or
wherein the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

78. The method of any one of claims 1-77, wherein the amplifying comprises multiplex amplification of two or more target nucleic acid sequences, and wherein the detecting step comprises multiplex detection of two or more nucleic acid amplification products derived from said two or more target nucleic acid sequences, optionally the two or more target nucleic acid sequences are specific to two or more different organisms, further optionally the two or more different organisms comprise one or more of SARS-CoV-2, Influenza A, Influenza B, and/or Influenza C.

79. The method of any one of claims 1-78, wherein the amplifying comprises one or more of Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA), optionally the amplifying does not comprise loop-mediated isothermal amplification (LAMP).

80. The method of any one of claims 1-79, wherein the method does not comprise one or more of: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

81. The method of any one of claims 1-80, wherein the method, the reagent composition, and/or the amplification reaction mixture does not comprise:
a template capable of generating the first quality control product other than the quality control template;
a probe capable of detecting of the first quality control product other than the signal-generating oligonucleotide;
a double-stranded template capable of generating the first quality control product;
a linear template capable of generating the first quality control product; and/or
a primer capable of hybridizing to the quality control template, the first quality control product and/or the second quality control product other than the quality control primer.

82. The method of any one of claims 1-81, wherein the method comprises determining the presence, absence and/or amount of the first quality control product.

83. The method of any one of claims 1-82, wherein the presence, absence and/or amount of the signal indicates:
the presence, absence and/or amount of the first quality control product;
the presence, absence and/or amount of one or more interfering components in the amplification reaction mixture;
(i) the integrity of the one or more amplification reagents in the amplification reaction mixture; (ii) failure of the instrument wherein the amplification reaction is conducted; and/or (iii) sample-derived inhibition of the amplification reaction, optionally sample-derived inhibition comprises matrix-derived inhibition; and/or
the degree to which the amplification of the target nucleic acid sequence is inhibited in the amplification reaction.

84. The method of any one of claims 1-83, wherein the method, the reagent composition, and/or the amplification reaction mixture comprises at least about 50,000 copies to about 10¹² copies of the quality control template, optionally a comparable method of monitoring an amplification reaction employs about 20 copies to about 100 copies of an internal control template.

85. The method of any one of claims 1-84, wherein the method, the reagent composition, and/or the amplification reaction mixture comprises an at least about 1.1-fold greater number of copies of the quality control template and/or quality control primer as compared to a comparable method of monitoring an amplification reaction that does not comprise the quality control template and/or the quality control primer, optionally said comparable method comprises an internal control template that is not capable of forming a hairpin structure.

86. The method of any one of claims 1-85, wherein a comparable method of monitoring an amplification reaction that does not comprise the quality control template and/or the quality control primer inhibits the amplification of the target nucleic acid sequence and/or the detection of nucleic acid amplification product by at least about 1.1-fold more than the method of any one of claims 1-85, optionally said comparable method comprises an internal control template that is not capable of forming a hairpin structure.

87. The method of any one of claims 1-86, wherein the quality control template and/or the quality control primer is not capable of hybridizing to the target nucleic acid sequence.

88. The method of any one of claims 1-87, wherein the presence of the quality control template and/or the quality control primer does not inhibit the amplification of the target nucleic acid sequence and/or the detection of nucleic acid amplification product.

89. The method of any one of claims 1-88, wherein the presence of the quality control template and/or the quality control primer in the amplification reaction mixture improves the amplification of the target nucleic acid sequence and/or detection of the nucleic acid amplification product by at least about 1.1-fold as compared to a comparable method wherein the quality control template and/or the quality control primer is absent from the amplification reaction mixture, optionally said comparable method comprises an internal control template that is not capable of forming a hairpin structure.

90. The method of any one of claims 1-89, wherein the number of false priming events and/or the generation of primer-dimers is reduced by at least about 1.1-fold as compared to a comparable method of monitoring an amplification reaction that does not comprise the quality control template and/or the quality control primer, optionally said comparable method comprises an internal control template that is not capable of forming a hairpin structure.

91. A kit for monitoring an amplification reaction, comprising
the quality control template of any one of claims 1-90;
the quality control primer of any one of claims 1-90;
the signal-generating oligonucleotide of any one of claims 1-90; and/or
the supplemental quality control primer of any one of claims 1-90.

92. The kit of any one of claim 91, comprising:
a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant; and/or
a reagent composition comprising one or more amplification reagents comprising one or more components for amplifying the target nucleic acid sequence under isothermal amplification conditions.

93. The kit of any one of claims 91-92, wherein said one or more components for amplifying comprise:
(i) a forward primer and a reverse primer, wherein the forward primer is capable of hybridizing to a sequence of a first strand of the target nucleic acid sequence, and the reverse primer is capable of hybridizing to a sequence of a second strand of the target nucleic acid sequence; and/or
(ii) an enzyme having a hyperthermophile polymerase activity capable of generating a nucleic acid amplification product, optionally the enzyme having a hyperthermophile polymerase activity has an amino acid sequence that is at least about 90% identical to the amino acid sequence of SEQ ID NO: 1 or a functional fragment thereof, optionally the enzyme having a hyperthermophile polymerase activity has an amino acid sequence that is at least about 95% identical to the amino acid sequence of SEQ ID NO: 1, further optionally the enzyme having a hyperthermophile polymerase activity is a polymerase comprising the amino acid sequence of SEQ ID NO: 1.

94. The kit of any one of claims 91-93, wherein the quality control template, the signal-generating oligonucleotide, the quality control primer, the supplemental quality control primer, and/or the one or more components for amplifying, are in a lyophilized or freeze-dried form and/or are present in the reagent composition.

95. A reaction mixture, comprising:
the quality control template of any one of claims 1-90;
the quality control primer of any one of claims 1-90;
the signal-generating oligonucleotide of any one of claims 1-90;
the supplemental quality control primer of any one of claims 1-90;
a target nucleic acid sequence; and/or
one or more additional primers and/or one or more probes specific to a target nucleic acid sequence.

96. The reaction mixture of claim 95, wherein the one or more additional primers and/or one or more probes specific to the target nucleic acid sequence comprise:
a forward primer and/or a reverse primer,
wherein the forward primer is capable of hybridizing to a sequence of a first strand of the target nucleic acid sequence, and the reverse primer is capable of hybridizing to a sequence of a second strand of the target nucleic acid sequence; and/or
a target signal-generating oligonucleotide capable of hybridizing to a nucleic acid amplification product generated by amplifying the target nucleic acid sequence.

97. The reaction mixture of any one of claims 95-96, comprising one or more of an enzyme having a polymerase activity, dNTPs, and a buffering agent, and optionally the enzyme is an enzyme having a hyperthermophile polymerase activity.

98. The reaction mixture of any one of claims 95-97, wherein the enzyme having a hyperthermophile polymerase activity has an amino acid sequence that is at least about 90% identical to the amino acid sequence of SEQ ID NO: 1 or a functional fragment thereof, and optionally the enzyme having a hyperthermophile polymerase activity comprises the amino acid sequence of SEQ ID NO: 1.


SEQUENCE LISTING (11 sequences)

SEQ ID NO: 1 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIDTLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - DNA, 12 bp, synthetic construct
  source 1..12: mol_type = other DNA
  modified_base 11: mod_base = OTHER; note = Phosphorothioated base
aaagatccctaa

SEQ ID NO: 4 - DNA, 25 bp, synthetic construct
  source 1..25: mol_type = other DNA
  modified_base 8: mod_base = OTHER; note = locked nucleic acid base
  modified_base 13: mod_base = OTHER; note = locked nucleic acid base
  modified_base 15: mod_base = OTHER; note = locked nucleic acid base
  modified_base 18: mod_base = OTHER; note = locked nucleic acid base
  modified_base 23: mod_base = OTHER; note = locked nucleic acid base
  modified_base 25: mod_base = OTHER; note = locked nucleic acid base
aaagatccctaacaccacttaggga

SEQ ID NO: 5 - DNA, 26 bp, synthetic construct
  source 1..26: mol_type = other DNA
agatccctaacaccacttagggatct

SEQ ID NO: 6 - DNA, 26 bp, synthetic construct
  source 1..26: mol_type = other DNA
agatccctaagtggtgttagggatct

SEQ ID NO: 7 - DNA, 30 bp, synthetic construct
  source 1..30: mol_type = other DNA
aaagatccctaagtggtgttagggatcttt

SEQ ID NO: 8 - DNA, 25 bp, synthetic construct
  source 1..25: mol_type = other DNA
  modified_base 13: mod_base = OTHER; note = locked nucleic acid base
  modified_base 15: mod_base = OTHER; note = locked nucleic acid base
  modified_base 18: mod_base = OTHER; note = locked nucleic acid base
  modified_base 20: mod_base = OTHER; note = locked nucleic acid base
  modified_base 23: mod_base = OTHER; note = locked nucleic acid base
  modified_base 25: mod_base = OTHER; note = locked nucleic acid base
aaagtaccctaacaccacttagggt

SEQ ID NO: 9 - DNA, 12 bp, synthetic construct
  source 1..12: mol_type = other DNA
  modified_base 11: mod_base = OTHER; note = Phosphorothioated base
aaagtaccctaa

SEQ ID NO: 10 - DNA, 30 bp, synthetic construct
  source 1..30: mol_type = other DNA
agatccctaacaccacttagggatctaaaa

SEQ ID NO: 11 - DNA, 25 bp, synthetic construct
  source 1..25: mol_type = other DNA
  modified_base 9: mod_base = OTHER; note = locked nucleic acid base
  modified_base 13: mod_base = OTHER; note = locked nucleic acid base
  modified_base 15: mod_base = OTHER; note = locked nucleic acid base
  modified_base 20: mod_base = OTHER; note = locked nucleic acid base
  modified_base 23: mod_base = OTHER; note = locked nucleic acid base
  modified_base 25: mod_base = OTHER; note = locked nucleic acid base
aaagtaccctaacaccacttagggt